SEQUENCE LISTING (44 sequences)

SEQ ID NO: 1 - protein, 50 aa, Araneus diadematus
SGCDVLVQALLEVVSALVSILGSSSIGQINYGASAQYTQMVGQSVAQALA

SEQ ID NO: 2 - protein, 30 aa, Araneus diadematus
SGCDVLVQALLEVVSALVSILGSSSIGQIN

SEQ ID NO: 3 - protein, 21 aa, Araneus diadematus
SGCDVLVQALLEVVSALVSIL

SEQ ID NO: 4 - protein, 1154 aa, Artificial Sequence
  recombinant spider silk protein ADF3KaiLargeNRSH1
MHHHHHHHHHHSSGSSLEVLFQGPARAGSGQQGPGQQGPGQQGPGQQGPYGPGASAAAAAAGGYGPGSGQQGPSQQGPGQQGPGGQGPYGPGASAAAAAAGGYGPGSGQQGPGGQGPYGPGSSAAAAAAGGNGPGSGQQGAGQQGPGQQGPGASAAAAAAGGYGPGSGQQGPGQQGPGGQGPYGPGASAAAAAAGGYGPGSGQGPGQQGPGGQGPYGPGASAAAAAAGGYGPGSGQQGPGQQGPGQQGPGGQGPYGPGASAAAAAAGGYGPGYGQQGPGQQGPGGQGPYGPGASAASAASGGYGPGSGQQGPGQQGPGGQGPYGPGASAAAAAAGGYGPGSGQQGPGQQGPGQQGPGQQGPGGQGPYGPGASAAAAAAGGYGPGSGQQGPGQQGPGQQGPGQQGPGQQGPGQQGPGQQGPGQQGPGQQGPGGQGAYGPGASAAAGAAGGYGPGSGQQGPGQQGPGQQGPGQQGPGQQGPGQQGPGQQGPGQQGPYGPGASAAAAAAGGYGPGSGQQGPGQQGPGQQGPGGQGPYGPGAASAAVSVSRARAGSGQQGPGQQGPGQQGPGQQGPYGPGASAAAAAAGGYGPGSGQQGPSQQGPGQQGPGGQGPYGPGASAAAAAAGGYGPGSGQQGPGGQGPYGPGSSAAAAAAGGNGPGSGQQGAGQQGPGQQGPGASAAAAAAGGYGPGSGQQGPGQQGPGGQGPYGPGASAAAAAAGGYGPGSGQGPGQQGPGGQGPYGPGASAAAAAAGGYGPGSGQQGPGQQGPGQQGPGGQGPYGPGASAAAAAAGGYGPGYGQQGPGQQGPGGQGPYGPGASAASAASGGYGPGSGQQGPGQQGPGGQGPYGPGASAAAAAAGGYGPGSGQQGPGQQGPGQQGPGQQGPGGQGPYGPGASAAAAAAGGYGPGSGQQGPGQQGPGQQGPGQQGPGQQGPGQQGPGQQGPGQQGPGQQGPGGQGAYGPGASAAAGAAGGYGPGSGQQGPGQQGPGQQGPGQQGPGQQGPGQQGPGQQGPGQQGPYGPGASAAAAAAGGYGPGSGQQGPGQQGPGQQGPGGQGPYGPGAASAAVSVGGYGPQSSSVPVASAVASRLSSPAASSRVSSAVSSLVSSGPTKHAALSNTISSVVSQVSASNPGLSGCDVLVQALLEVVSALVSIL

SEQ ID NO: 5 - protein, 24 aa, Artificial Sequence
  His tag and start codon
MHHHHHHHHHHSSGSSLEVLFQGP

SEQ ID NO: 6 - protein, 597 aa, Artificial Sequence
  Met-PRT380
MGPGQQGPYGPGASAAAAAGQNGPGSGQQGPGQSAAAAAGQYGPGQQGPGQQGPGSSAAAAAGPGQYGPGQQGPSASAAAAAGPGSGQQGPGASAAAAAGQYGPGQQGPGQQGPGSSAAAAAGQYGSGPGQQGPYGSAAAAAGPGSGQYGQGPYGPGASAAAAAGPGQYGPGQQGPSASAAAAAGSGQQGPGQYGPYASAAAAAGQYGSGPGQQGPYGPGQSAAAAAGSGQQGPGQQGPYASAAAAAGPGQQGPYGPGSSAAAAAGQYGYGPGQQGPYGPGASAAAAAGQNGPGSGQYGPGQQGPGQSAAAAAGPGQQGPYGPGASAAAAAGQYGPGQQGPGQYGPGSSAAAAAGPGQQGPYGPGSSAAAAAGQYGPGQQGPYGPGQSAAAAAGQYQQGPGQQGPYGPGASAAAAAGPGQQGPYGPGASAAAAAGPGQYGPGQQGPSASAAAAAGQYGSGPGQYGPYGPGQSAAAAAGPGSGQQGQGPYGPGASAAAAAGQYGPGQQGPYGPGQSAAAAAGPGSGQYGPGASAAAAAGQNGPGSGQYGPGQQGPGQSAAAAAGQYQQGPGQQGPYGPGASAAAAAGPGSGQQGPGAS

SEQ ID NO: 7 - protein, 590 aa, Artificial Sequence
  Met-PRT410
MGPGQQGPYGPGASAAAAAGQNGPGSGQQGPGQSGQYGPGQQGPGQQGPGSSAAAAAGPGQYGPGQQGPSASAAAAAGPGSGQQGPGASGQYGPGQQGPGQQGPGSSAAAAAGQYGSGPGQQGPYGSAAAAAGPGSGQYGQGPYGPGASGPGQYGPGQQGPSASAAAAAGSGQQGPGQYGPYASAAAAAGQYGSGPGQQGPYGPGQSGSGQQGPGQQGPYASAAAAAGPGQQGPYGPGSSAAAAAGQYGYGPGQQGPYGPGASGQNGPGSGQYGPGQQGPGQSAAAAAGPGQQGPYGPGASAAAAAGQYGPGQQGPGQYGPGSSGPGQQGPYGPGSSAAAAAGQYGPGQQGPYGPGQSAAAAAGQYQQGPGQQGPYGPGASGPGQQGPYGPGASAAAAAGPGQYGPGQQGPSASAAAAAGQYGSGPGQYGPYGPGQSGPGSGQQGQGPYGPGASAAAAAGQYGPGQQGPYGPGQSAAAAAGPGSGQYGPGASGQNGPGSGQYGPGQQGPGQSAAAAAGQYQQGPGQQGPYGPGASAAAAAGQYGSGPGQQGPYGPGQSGSGQQGPGQQGPYASAAAAAGPGSGQQGPGAS

SEQ ID NO: 8 - protein, 565 aa, Artificial Sequence
  Met-PRT525
MGPGQQGPYGPGASAAAAAAAGSNGPGSGQQGPGQSGQYGPGQQGPGQQGPGSSAAAAAAAGPGQYGPGQQGPSASAAAAAAAGPGSGQQGPGASGQYGPGQQGPGQQGPGSSAAAAAAAGSYGSGPGQQGPYGSAAAAAAAGPGSGQYGQGPYGPGASGPGQYGPGQQGPSASAAAAAAAGSGQQGPGQYGPYASAAAAAAAGSYGSGPGQQGPYGPGQSGSGQQGPGQQGPYASAAAAAAAGPGQQGPYGPGSSAAAAAAAGSYGYGPGQQGPYGPGASGQNGPGSGQYGPGQQGPGPSAAAAAAAGPGQQGPYGPGASAAAAAAAGSYGPGQQGPGQYGPGSSGPGQQGPYGPGSSAAAAAAAGSYGPGQQGPYGPGPSAAAAAAAGSYQQGPGQQGPYGPGASGPGQQGPYGPGASAAAAAAAGPGQYGPGQQGPSASAAAAAAAGSYGSGPGQYGPYGPGQSGPGSGQQGQGPYGPGASAAAAAAAGSYGPGQQGPYGPGPSAAAAAAAGPGSGQYGPGASGQNGPGSGQYGPGQQGPGPSAAAAAAAGPGSGQQGPGAS

SEQ ID NO: 9 - protein, 2364 aa, Artificial Sequence
  Met-PRT799
MGPGQQGPYGPGASAAAAAGQNGPGSGQQGPGQSGQYGPGQQGPGQQGPGSSAAAAAGPGQYGPGQQGPSASAAAAAGPGSGQQGPGASGQYGPGQQGPGQQGPGSSAAAAAGQYGSGPGQQGPYGSAAAAAGPGSGQYGQGPYGPGASGPGQYGPGQQGPSASAAAAAGSGQQGPGQYGPYASAAAAAGQYGSGPGQQGPYGPGQSGSGQQGPGQQGPYASAAAAAGPGQQGPYGPGSSAAAAAGQYGYGPGQQGPYGPGASGQNGPGSGQYGPGQQGPGQSAAAAAGPGQQGPYGPGASAAAAAGQYGPGQQGPGQYGPGSSGPGQQGPYGPGSSAAAAAGQYGPGQQGPYGPGQSAAAAAGQYQQGPGQQGPYGPGASGPGQQGPYGPGASAAAAAGPGQYGPGQQGPSASAAAAAGQYGSGPGQYGPYGPGQSGPGSGQQGQGPYGPGASAAAAAGQYGPGQQGPYGPGQSAAAAAGPGSGQYGPGASGQNGPGSGQYGPGQQGPGQSAAAAAGQYQQGPGQQGPYGPGASAAAAAGQYGSGPGQQGPYGPGQSGSGQQGPGQQGPYASAAAAAGPGSGQQGPGASGQQGPYGPGASAAAAAGQNGPGSGQQGPGQSGQYGPGQQGPGQQGPGSSAAAAAGPGQYGPGQQGPSASAAAAAGPGSGQQGPGASGQYGPGQQGPGQQGPGSSAAAAAGQYGSGPGQQGPYGSAAAAAGPGSGQYGQGPYGPGASGPGQYGPGQQGPSASAAAAAGSGQQGPGQYGPYASAAAAAGQYGSGPGQQGPYGPGQSGSGQQGPGQQGPYASAAAAAGPGQQGPYGPGSSAAAAAGQYGYGPGQQGPYGPGASGQNGPGSGQYGPGQQGPGQSAAAAAGPGQQGPYGPGASAAAAAGQYGPGQQGPGQYGPGSSGPGQQGPYGPGSSAAAAAGQYGPGQQGPYGPGQSAAAAAGQYQQGPGQQGPYGPGASGPGQQGPYGPGASAAAAAGPGQYGPGQQGPSASAAAAAGQYGSGPGQYGPYGPGQSGPGSGQQGQGPYGPGASAAAAAGQYGPGQQGPYGPGQSAAAAAGPGSGQYGPGASGQNGPGSGQYGPGQQGPGQSAAAAAGQYQQGPGQQGPYGPGASAAAAAGQYGSGPGQQGPYGPGQSGSGQQGPGQQGPYASAAAAAGPGSGQQGPGASGQQGPYGPGASAAAAAGQNGPGSGQQGPGQSGQYGPGQQGPGQQGPGSSAAAAAGPGQYGPGQQGPSASAAAAAGPGSGQQGPGASGQYGPGQQGPGQQGPGSSAAAAAGQYGSGPGQQGPYGSAAAAAGPGSGQYGQGPYGPGASGPGQYGPGQQGPSASAAAAAGSGQQGPGQYGPYASAAAAAGQYGSGPGQQGPYGPGQSGSGQQGPGQQGPYASAAAAAGPGQQGPYGPGSSAAAAAGQYGYGPGQQGPYGPGASGQNGPGSGQYGPGQQGPGQSAAAAAGPGQQGPYGPGASAAAAAGQYGPGQQGPGQYGPGSSGPGQQGPYGPGSSAAAAAGQYGPGQQGPYGPGQSAAAAAGQYQQGPGQQGPYGPGASGPGQQGPYGPGASAAAAAGPGQYGPGQQGPSASAAAAAGQYGSGPGQYGPYGPGQSGPGSGQQGQGPYGPGASAAAAAGQYGPGQQGPYGPGQSAAAAAGPGSGQYGPGASGQNGPGSGQYGPGQQGPGQSAAAAAGQYQQGPGQQGPYGPGASAAAAAGQYGSGPGQQGPYGPGQSGSGQQGPGQQGPYASAAAAAGPGSGQQGPGASGQQGPYGPGASAAAAAGQNGPGSGQQGPGQSGQYGPGQQGPGQQGPGSSAAAAAGPGQYGPGQQGPSASAAAAAGPGSGQQGPGASGQYGPGQQGPGQQGPGSSAAAAAGQYGSGPGQQGPYGSAAAAAGPGSGQYGQGPYGPGASGPGQYGPGQQGPSASAAAAAGSGQQGPGQYGPYASAAAAAGQYGSGPGQQGPYGPGQSGSGQQGPGQQGPYASAAAAAGPGQQGPYGPGSSAAAAAGQYGYGPGQQGPYGPGASGQNGPGSGQYGPGQQGPGQSAAAAAGPGQQGPYGPGASAAAAAGQYGPGQQGPGQYGPGSSGPGQQGPYGPGSSAAAAAGQYGPGQQGPYGPGQSAAAAAGQYQQGPGQQGPYGPGASGPGQQGPYGPGASAAAAAGPGQYGPGQQGPSASAAAAAGQYGSGPGQYGPYGPGQSGPGSGQQGQGPYGPGASAAAAAGQYGPGQQGPYGPGQSAAAAAGPGSGQYGPGASGQNGPGSGQYGPGQQGPGQSAAAAAGQYQQGPGQQGPYGPGASAAAAAGQYGSGPGQQGPYGPGQSGSGQQGPGQQGPYASAAAAAGPGSGQQGSSVDKLAAALEHHHHHH

SEQ ID NO: 10 - protein, 597 aa, Artificial Sequence
  Met-PRT313
MGPGGQGPYGPGASAAAAAGGNGPGSGQQGPGGSAAAAAGGYGPGGQGPGQQGPGSSAAAAAGPGGYGPGGQGPSASAAAAAGPGSGQQGPGASAAAAAGGYGPGGQGPGQQGPGSSAAAAAGGYGSGPGQQGPYGSAAAAAGPGSGGYGQGPYGPGASAAAAAGPGGYGPGGQGPSASAAAAAGSGQQGPGGYGPYASAAAAAGGYGSGPGQQGPYGPGGSAAAAAGSGQQGPGQQGPYASAAAAAGPGGQGPYGPGSSAAAAAGGYGYGPGGQGPYGPGASAAAAAGGNGPGSGGYGPGQQGPGGSAAAAAGPGGQGPYGPGASAAAAAGGYGPGGQGPGGYGPGSSAAAAAGPGGQGPYGPGSSAAAAAGGYGPGQQGPYGPGGSAAAAAGGYQQGPGGQGPYGPGASAAAAAGPGGQGPYGPGASAAAAAGPGGYGPGGQGPSASAAAAAGGYGSGPGGYGPYGPGGSAAAAAGPGSGQQGQGPYGPGASAAAAAGGYGPGQQGPYGPGGSAAAAAGPGSGGYGPGASAAAAAGGNGPGSGGYGPGQQGPGGSAAAAAGGYQQGPGGQGPYGPGASAAAAAGPGSGQQGPGAS

SEQ ID NO: 11 - protein, 12 aa, Artificial Sequence
  HisTag
MHHHHHHSSGSS

SEQ ID NO: 12 - protein, 608 aa, Artificial Sequence
  PRT380
MHHHHHHSSGSSGPGQQGPYGPGASAAAAAGQNGPGSGQQGPGQSAAAAAGQYGPGQQGPGQQGPGSSAAAAAGPGQYGPGQQGPSASAAAAAGPGSGQQGPGASAAAAAGQYGPGQQGPGQQGPGSSAAAAAGQYGSGPGQQGPYGSAAAAAGPGSGQYGQGPYGPGASAAAAAGPGQYGPGQQGPSASAAAAAGSGQQGPGQYGPYASAAAAAGQYGSGPGQQGPYGPGQSAAAAAGSGQQGPGQQGPYASAAAAAGPGQQGPYGPGSSAAAAAGQYGYGPGQQGPYGPGASAAAAAGQNGPGSGQYGPGQQGPGQSAAAAAGPGQQGPYGPGASAAAAAGQYGPGQQGPGQYGPGSSAAAAAGPGQQGPYGPGSSAAAAAGQYGPGQQGPYGPGQSAAAAAGQYQQGPGQQGPYGPGASAAAAAGPGQQGPYGPGASAAAAAGPGQYGPGQQGPSASAAAAAGQYGSGPGQYGPYGPGQSAAAAAGPGSGQQGQGPYGPGASAAAAAGQYGPGQQGPYGPGQSAAAAAGPGSGQYGPGASAAAAAGQNGPGSGQYGPGQQGPGQSAAAAAGQYQQGPGQQGPYGPGASAAAAAGPGSGQQGPGAS

SEQ ID NO: 13 - protein, 601 aa, Artificial Sequence
  PRT410
MHHHHHHSSGSSGPGQQGPYGPGASAAAAAGQNGPGSGQQGPGQSGQYGPGQQGPGQQGPGSSAAAAAGPGQYGPGQQGPSASAAAAAGPGSGQQGPGASGQYGPGQQGPGQQGPGSSAAAAAGQYGSGPGQQGPYGSAAAAAGPGSGQYGQGPYGPGASGPGQYGPGQQGPSASAAAAAGSGQQGPGQYGPYASAAAAAGQYGSGPGQQGPYGPGQSGSGQQGPGQQGPYASAAAAAGPGQQGPYGPGSSAAAAAGQYGYGPGQQGPYGPGASGQNGPGSGQYGPGQQGPGQSAAAAAGPGQQGPYGPGASAAAAAGQYGPGQQGPGQYGPGSSGPGQQGPYGPGSSAAAAAGQYGPGQQGPYGPGQSAAAAAGQYQQGPGQQGPYGPGASGPGQQGPYGPGASAAAAAGPGQYGPGQQGPSASAAAAAGQYGSGPGQYGPYGPGQSGPGSGQQGQGPYGPGASAAAAAGQYGPGQQGPYGPGQSAAAAAGPGSGQYGPGASGQNGPGSGQYGPGQQGPGQSAAAAAGQYQQGPGQQGPYGPGASAAAAAGQYGSGPGQQGPYGPGQSGSGQQGPGQQGPYASAAAAAGPGSGQQGPGAS

SEQ ID NO: 14 - protein, 576 aa, Artificial Sequence
  PRT525
MHHHHHHSSGSSGPGQQGPYGPGASAAAAAAAGSNGPGSGQQGPGQSGQYGPGQQGPGQQGPGSSAAAAAAAGPGQYGPGQQGPSASAAAAAAAGPGSGQQGPGASGQYGPGQQGPGQQGPGSSAAAAAAAGSYGSGPGQQGPYGSAAAAAAAGPGSGQYGQGPYGPGASGPGQYGPGQQGPSASAAAAAAAGSGQQGPGQYGPYASAAAAAAAGSYGSGPGQQGPYGPGQSGSGQQGPGQQGPYASAAAAAAAGPGQQGPYGPGSSAAAAAAAGSYGYGPGQQGPYGPGASGQNGPGSGQYGPGQQGPGPSAAAAAAAGPGQQGPYGPGASAAAAAAAGSYGPGQQGPGQYGPGSSGPGQQGPYGPGSSAAAAAAAGSYGPGQQGPYGPGPSAAAAAAAGSYQQGPGQQGPYGPGASGPGQQGPYGPGASAAAAAAAGPGQYGPGQQGPSASAAAAAAAGSYGSGPGQYGPYGPGQSGPGSGQQGQGPYGPGASAAAAAAAGSYGPGQQGPYGPGPSAAAAAAAGPGSGQYGPGASGQNGPGSGQYGPGQQGPGPSAAAAAAAGPGSGQQGPGAS

SEQ ID NO: 15 - protein, 2375 aa, Artificial Sequence
  PRT799
MHHHHHHSSGSSGPGQQGPYGPGASAAAAAGQNGPGSGQQGPGQSGQYGPGQQGPGQQGPGSSAAAAAGPGQYGPGQQGPSASAAAAAGPGSGQQGPGASGQYGPGQQGPGQQGPGSSAAAAAGQYGSGPGQQGPYGSAAAAAGPGSGQYGQGPYGPGASGPGQYGPGQQGPSASAAAAAGSGQQGPGQYGPYASAAAAAGQYGSGPGQQGPYGPGQSGSGQQGPGQQGPYASAAAAAGPGQQGPYGPGSSAAAAAGQYGYGPGQQGPYGPGASGQNGPGSGQYGPGQQGPGQSAAAAAGPGQQGPYGPGASAAAAAGQYGPGQQGPGQYGPGSSGPGQQGPYGPGSSAAAAAGQYGPGQQGPYGPGQSAAAAAGQYQQGPGQQGPYGPGASGPGQQGPYGPGASAAAAAGPGQYGPGQQGPSASAAAAAGQYGSGPGQYGPYGPGQSGPGSGQQGQGPYGPGASAAAAAGQYGPGQQGPYGPGQSAAAAAGPGSGQYGPGASGQNGPGSGQYGPGQQGPGQSAAAAAGQYQQGPGQQGPYGPGASAAAAAGQYGSGPGQQGPYGPGQSGSGQQGPGQQGPYASAAAAAGPGSGQQGPGASGQQGPYGPGASAAAAAGQNGPGSGQQGPGQSGQYGPGQQGPGQQGPGSSAAAAAGPGQYGPGQQGPSASAAAAAGPGSGQQGPGASGQYGPGQQGPGQQGPGSSAAAAAGQYGSGPGQQGPYGSAAAAAGPGSGQYGQGPYGPGASGPGQYGPGQQGPSASAAAAAGSGQQGPGQYGPYASAAAAAGQYGSGPGQQGPYGPGQSGSGQQGPGQQGPYASAAAAAGPGQQGPYGPGSSAAAAAGQYGYGPGQQGPYGPGASGQNGPGSGQYGPGQQGPGQSAAAAAGPGQQGPYGPGASAAAAAGQYGPGQQGPGQYGPGSSGPGQQGPYGPGSSAAAAAGQYGPGQQGPYGPGQSAAAAAGQYQQGPGQQGPYGPGASGPGQQGPYGPGASAAAAAGPGQYGPGQQGPSASAAAAAGQYGSGPGQYGPYGPGQSGPGSGQQGQGPYGPGASAAAAAGQYGPGQQGPYGPGQSAAAAAGPGSGQYGPGASGQNGPGSGQYGPGQQGPGQSAAAAAGQYQQGPGQQGPYGPGASAAAAAGQYGSGPGQQGPYGPGQSGSGQQGPGQQGPYASAAAAAGPGSGQQGPGASGQQGPYGPGASAAAAAGQNGPGSGQQGPGQSGQYGPGQQGPGQQGPGSSAAAAAGPGQYGPGQQGPSASAAAAAGPGSGQQGPGASGQYGPGQQGPGQQGPGSSAAAAAGQYGSGPGQQGPYGSAAAAAGPGSGQYGQGPYGPGASGPGQYGPGQQGPSASAAAAAGSGQQGPGQYGPYASAAAAAGQYGSGPGQQGPYGPGQSGSGQQGPGQQGPYASAAAAAGPGQQGPYGPGSSAAAAAGQYGYGPGQQGPYGPGASGQNGPGSGQYGPGQQGPGQSAAAAAGPGQQGPYGPGASAAAAAGQYGPGQQGPGQYGPGSSGPGQQGPYGPGSSAAAAAGQYGPGQQGPYGPGQSAAAAAGQYQQGPGQQGPYGPGASGPGQQGPYGPGASAAAAAGPGQYGPGQQGPSASAAAAAGQYGSGPGQYGPYGPGQSGPGSGQQGQGPYGPGASAAAAAGQYGPGQQGPYGPGQSAAAAAGPGSGQYGPGASGQNGPGSGQYGPGQQGPGQSAAAAAGQYQQGPGQQGPYGPGASAAAAAGQYGSGPGQQGPYGPGQSGSGQQGPGQQGPYASAAAAAGPGSGQQGPGASGQQGPYGPGASAAAAAGQNGPGSGQQGPGQSGQYGPGQQGPGQQGPGSSAAAAAGPGQYGPGQQGPSASAAAAAGPGSGQQGPGASGQYGPGQQGPGQQGPGSSAAAAAGQYGSGPGQQGPYGSAAAAAGPGSGQYGQGPYGPGASGPGQYGPGQQGPSASAAAAAGSGQQGPGQYGPYASAAAAAGQYGSGPGQQGPYGPGQSGSGQQGPGQQGPYASAAAAAGPGQQGPYGPGSSAAAAAGQYGYGPGQQGPYGPGASGQNGPGSGQYGPGQQGPGQSAAAAAGPGQQGPYGPGASAAAAAGQYGPGQQGPGQYGPGSSGPGQQGPYGPGSSAAAAAGQYGPGQQGPYGPGQSAAAAAGQYQQGPGQQGPYGPGASGPGQQGPYGPGASAAAAAGPGQYGPGQQGPSASAAAAAGQYGSGPGQYGPYGPGQSGPGSGQQGQGPYGPGASAAAAAGQYGPGQQGPYGPGQSAAAAAGPGSGQYGPGASGQNGPGSGQYGPGQQGPGQSAAAAAGQYQQGPGQQGPYGPGASAAAAAGQYGSGPGQQGPYGPGQSGSGQQGPGQQGPYASAAAAAGPGSGQQGSSVDKLAAALEHHHHHH

SEQ ID NO: 16 - protein, 608 aa, Artificial Sequence
  PRT313
MHHHHHHSSGSSGPGGQGPYGPGASAAAAAGGNGPGSGQQGPGGSAAAAAGGYGPGGQGPGQQGPGSSAAAAAGPGGYGPGGQGPSASAAAAAGPGSGQQGPGASAAAAAGGYGPGGQGPGQQGPGSSAAAAAGGYGSGPGQQGPYGSAAAAAGPGSGGYGQGPYGPGASAAAAAGPGGYGPGGQGPSASAAAAAGSGQQGPGGYGPYASAAAAAGGYGSGPGQQGPYGPGGSAAAAAGSGQQGPGQQGPYASAAAAAGPGGQGPYGPGSSAAAAAGGYGYGPGGQGPYGPGASAAAAAGGNGPGSGGYGPGQQGPGGSAAAAAGPGGQGPYGPGASAAAAAGGYGPGGQGPGGYGPGSSAAAAAGPGGQGPYGPGSSAAAAAGGYGPGQQGPYGPGGSAAAAAGGYQQGPGGQGPYGPGASAAAAAGPGGQGPYGPGASAAAAAGPGGYGPGGQGPSASAAAAAGGYGSGPGGYGPYGPGGSAAAAAGPGSGQQGQGPYGPGASAAAAAGGYGPGQQGPYGPGGSAAAAAGPGSGGYGPGASAAAAAGGNGPGSGGYGPGQQGPGGSAAAAAGGYQQGPGGQGPYGPGASAAAAAGPGSGQQGPGAS

SEQ ID NO: 17 - protein, 590 aa, Artificial Sequence
  Met-PRT399
MGPGGQGPYGPGASAAAAAGGNGPGSGQQGPGGSGGYGPGGQGPGQQGPGSSAAAAAGPGGYGPGGQGPSASAAAAAGPGSGQQGPGASGGYGPGGQGPGQQGPGSSAAAAAGGYGSGPGQQGPYGSAAAAAGPGSGGYGQGPYGPGASGPGGYGPGGQGPSASAAAAAGSGQQGPGGYGPYASAAAAAGGYGSGPGQQGPYGPGGSGSGQQGPGQQGPYASAAAAAGPGGQGPYGPGSSAAAAAGGYGYGPGGQGPYGPGASGGNGPGSGGYGPGQQGPGGSAAAAAGPGGQGPYGPGASAAAAAGGYGPGGQGPGGYGPGSSGPGGQGPYGPGSSAAAAAGGYGPGQQGPYGPGGSAAAAAGGYQQGPGGQGPYGPGASGPGGQGPYGPGASAAAAAGPGGYGPGGQGPSASAAAAAGGYGSGPGGYGPYGPGGSGPGSGQQGQGPYGPGASAAAAAGGYGPGQQGPYGPGGSAAAAAGPGSGGYGPGASGGNGPGSGGYGPGQQGPGGSAAAAAGGYQQGPGGQGPYGPGASAAAAAGGYGSGPGQQGPYGPGGSGSGQQGPGQQGPYASAAAAAGPGSGQQGPGAS

SEQ ID NO: 18 - protein, 601 aa, Artificial Sequence
  PRT399
MHHHHHHSSGSSGPGGQGPYGPGASAAAAAGGNGPGSGQQGPGGSGGYGPGGQGPGQQGPGSSAAAAAGPGGYGPGGQGPSASAAAAAGPGSGQQGPGASGGYGPGGQGPGQQGPGSSAAAAAGGYGSGPGQQGPYGSAAAAAGPGSGGYGQGPYGPGASGPGGYGPGGQGPSASAAAAAGSGQQGPGGYGPYASAAAAAGGYGSGPGQQGPYGPGGSGSGQQGPGQQGPYASAAAAAGPGGQGPYGPGSSAAAAAGGYGYGPGGQGPYGPGASGGNGPGSGGYGPGQQGPGGSAAAAAGPGGQGPYGPGASAAAAAGGYGPGGQGPGGYGPGSSGPGGQGPYGPGSSAAAAAGGYGPGQQGPYGPGGSAAAAAGGYQQGPGGQGPYGPGASGPGGQGPYGPGASAAAAAGPGGYGPGGQGPSASAAAAAGGYGSGPGGYGPYGPGGSGPGSGQQGQGPYGPGASAAAAAGGYGPGQQGPYGPGGSAAAAAGPGSGGYGPGASGGNGPGSGGYGPGQQGPGGSAAAAAGGYQQGPGGQGPYGPGASAAAAAGGYGSGPGQQGPYGPGGSGSGQQGPGQQGPYASAAAAAGPGSGQQGPGAS

SEQ ID NO: 19 - protein, 612 aa, Artificial Sequence
  Met-PRT720
MGPGQQGPYGPGASAAAAAGQNGPGSGQQGPGQSGQYGPGQQGPGQQGPGSSAAAAAGPGQYVLIGPGQQVLIGPSASAAAAAGPGSGQQGPGASGQYGPGQQGPGQQGPGSSAAAAAGSYGSVLIGPGQQVLIGPYGSAAAAAGPGSGQYGQGPYGPGASGPGQYGPGQQGPSASAAAAAGSGQQVLIGPGQYVLIGPYASAAAAAGQYGSGPGQQGPYGPGQSGSGQQGPGQQGPYASAAAAAGPGQQVLIGPYVLIGPGSSAAAAAGQYGYGPGQQGPYGPGASGQNGPGSGQYGPGQQGPGQSAAAAAGPGQQVLIGPYVLIGPGASAAAAAGQYGPGQQGPGQYGPGSSGPGQQGPYGPGSSAAAAAGSYGPGQQVLIGPYVLIGPGPSAAAAAGQYQQGPGQQGPYGPGASGPGQQGPYGPGASAAAAAGPGQYVLIGPGQQVLIGPSASAAAAAGQYGSGPGQYGPYGPGQSGPGSGQQGQGPYGPGASAAAAAGSYGPGQQVLIGPYVLIGPGPSAAAAAGPGSGQYGPGASGQNGPGSGQYGPGQQGPGQSAAAAAGQYQQVLIGPGQQGPYVLIGPGASAAAAAGPGSGQQVLIGPGASVLI

SEQ ID NO: 20 - protein, 592 aa, Artificial Sequence
  Met-PRT665
MGPGQQGPYGPGASAAAAAAAGSNGPGSGQQGPGQSGQYGPGQQGPGQQGPGSSAAAAAAAGPGQYVLIGPGQQGPSASAAAAAAAGPGSGQQGPGASGQYGPGQQGPGQQGPGSSAAAAAAAGSYGSVLIGPGQQGPYGSAAAAAAAGPGSGQYGQGPYGPGASGPGQYGPGQQGPSASAAAAAAAGSGQQVLIGPGQYGPYASAAAAAAAGSYGSGPGQQGPYGPGQSGSGQQGPGQQGPYASAAAAAAAGPGQQVLIGPYGPGSSAAAAAAAGSYGYGPGQQGPYGPGASGQNGPGSGQYGPGQQGPGPSAAAAAAAGPGQQVLIGPYGPGASAAAAAAAGSYGPGQQGPGQYGPGSSGPGQQGPYGPGSSAAAAAAAGSYGPGQQVLIGPYGPGPSAAAAAAAGSYQQGPGQQGPYGPGASGPGQQGPYGPGASAAAAAAAGPGQYVLIGPGQQGPSASAAAAAAAGSYGSGPGQYGPYGPGQSGPGSGQQGQGPYGPGASAAAAAAAGSYGPGQQVLIGPYGPGPSAAAAAAAGPGSGQYGPGASGQNGPGSGQYGPGQQGPGPSAAAAAAAGPGSGQQGPGASVLI

SEQ ID NO: 21 - protein, 619 aa, Artificial Sequence
  Met-PRT666
MGPGQQGPYGPGASAAAAAAAGSNGPGSGQQGPGQSGQYGPGQQGPGQQGPGSSAAAAAAAGPGQYVLIGPGQQVLIGPSASAAAAAAAGPGSGQQGPGASGQYGPGQQGPGQQGPGSSAAAAAAAGSYGSVLIGPGQQVLIGPYGSAAAAAAAGPGSGQYGQGPYGPGASGPGQYGPGQQGPSASAAAAAAAGSGQQVLIGPGQYVLIGPYASAAAAAAAGSYGSGPGQQGPYGPGQSGSGQQGPGQQGPYASAAAAAAAGPGQQVLIGPYVLIGPGSSAAAAAAAGSYGYGPGQQGPYGPGASGQNGPGSGQYGPGQQGPGPSAAAAAAAGPGQQVLIGPYVLIGPGASAAAAAAAGSYGPGQQGPGQYGPGSSGPGQQGPYGPGSSAAAAAAAGSYGPGQQVLIGPYVLIGPGPSAAAAAAAGSYQQGPGQQGPYGPGASGPGQQGPYGPGASAAAAAAAGPGQYVLIGPGQQVLIGPSASAAAAAAAGSYGSGPGQYGPYGPGQSGPGSGQQGQGPYGPGASAAAAAAAGSYGPGQQVLIGPYVLIGPGPSAAAAAAAGPGSGQYGPGASGQNGPGSGQYGPGQQGPGPSAAAAAAAGPGSGQQVLIGPGASVLI

SEQ ID NO: 22 - protein, 623 aa, Artificial Sequence
  PRT720
MHHHHHHSSGSSGPGQQGPYGPGASAAAAAGQNGPGSGQQGPGQSGQYGPGQQGPGQQGPGSSAAAAAGPGQYVLIGPGQQVLIGPSASAAAAAGPGSGQQGPGASGQYGPGQQGPGQQGPGSSAAAAAGSYGSVLIGPGQQVLIGPYGSAAAAAGPGSGQYGQGPYGPGASGPGQYGPGQQGPSASAAAAAGSGQQVLIGPGQYVLIGPYASAAAAAGQYGSGPGQQGPYGPGQSGSGQQGPGQQGPYASAAAAAGPGQQVLIGPYVLIGPGSSAAAAAGQYGYGPGQQGPYGPGASGQNGPGSGQYGPGQQGPGQSAAAAAGPGQQVLIGPYVLIGPGASAAAAAGQYGPGQQGPGQYGPGSSGPGQQGPYGPGSSAAAAAGSYGPGQQVLIGPYVLIGPGPSAAAAAGQYQQGPGQQGPYGPGASGPGQQGPYGPGASAAAAAGPGQYVLIGPGQQVLIGPSASAAAAAGQYGSGPGQYGPYGPGQSGPGSGQQGQGPYGPGASAAAAAGSYGPGQQVLIGPYVLIGPGPSAAAAAGPGSGQYGPGASGQNGPGSGQYGPGQQGPGQSAAAAAGQYQQVLIGPGQQGPYVLIGPGASAAAAAGPGSGQQVLIGPGASVLI

SEQ ID NO: 23 - protein, 603 aa, Artificial Sequence
  PRT665
MHHHHHHSSGSSGPGQQGPYGPGASAAAAAAAGSNGPGSGQQGPGQSGQYGPGQQGPGQQGPGSSAAAAAAAGPGQYVLIGPGQQGPSASAAAAAAAGPGSGQQGPGASGQYGPGQQGPGQQGPGSSAAAAAAAGSYGSVLIGPGQQGPYGSAAAAAAAGPGSGQYGQGPYGPGASGPGQYGPGQQGPSASAAAAAAAGSGQQVLIGPGQYGPYASAAAAAAAGSYGSGPGQQGPYGPGQSGSGQQGPGQQGPYASAAAAAAAGPGQQVLIGPYGPGSSAAAAAAAGSYGYGPGQQGPYGPGASGQNGPGSGQYGPGQQGPGPSAAAAAAAGPGQQVLIGPYGPGASAAAAAAAGSYGPGQQGPGQYGPGSSGPGQQGPYGPGSSAAAAAAAGSYGPGQQVLIGPYGPGPSAAAAAAAGSYQQGPGQQGPYGPGASGPGQQGPYGPGASAAAAAAAGPGQYVLIGPGQQGPSASAAAAAAAGSYGSGPGQYGPYGPGQSGPGSGQQGQGPYGPGASAAAAAAAGSYGPGQQVLIGPYGPGPSAAAAAAAGPGSGQYGPGASGQNGPGSGQYGPGQQGPGPSAAAAAAAGPGSGQQGPGASVLI

SEQ ID NO: 24 - protein, 630 aa, Artificial Sequence
  PRT666
MHHHHHHSSGSSGPGQQGPYGPGASAAAAAAAGSNGPGSGQQGPGQSGQYGPGQQGPGQQGPGSSAAAAAAAGPGQYVLIGPGQQVLIGPSASAAAAAAAGPGSGQQGPGASGQYGPGQQGPGQQGPGSSAAAAAAAGSYGSVLIGPGQQVLIGPYGSAAAAAAAGPGSGQYGQGPYGPGASGPGQYGPGQQGPSASAAAAAAAGSGQQVLIGPGQYVLIGPYASAAAAAAAGSYGSGPGQQGPYGPGQSGSGQQGPGQQGPYASAAAAAAAGPGQQVLIGPYVLIGPGSSAAAAAAAGSYGYGPGQQGPYGPGASGQNGPGSGQYGPGQQGPGPSAAAAAAAGPGQQVLIGPYVLIGPGASAAAAAAAGSYGPGQQGPGQYGPGSSGPGQQGPYGPGSSAAAAAAAGSYGPGQQVLIGPYVLIGPGPSAAAAAAAGSYQQGPGQQGPYGPGASGPGQQGPYGPGASAAAAAAAGPGQYVLIGPGQQVLIGPSASAAAAAAAGSYGSGPGQYGPYGPGQSGPGSGQQGQGPYGPGASAAAAAAAGSYGPGQQVLIGPYVLIGPGPSAAAAAAAGPGSGQYGPGASGQNGPGSGQYGPGQQGPGPSAAAAAAAGPGSGQQVLIGPGASVLI

SEQ ID NO: 25 - protein, 593 aa, Artificial Sequence
  Met-PRT888
MGSSGPGVLGPYGPGASAAAAAGQNGPGSGVLGPGQSGQYGPGVLGPGVLGPGSSAAAAAGPGQYGPGVLGPSASAAAAAGPGSGVLGPGASGQYGPGVLGPGVLGPGSSAAAAAGQYGSGPGVLGPYGSAAAAAGPGSGQYGQGPYGPGASGPGQYGPGVLGPSASAAAAAGSGVLGPGQYGPYASAAAAAGQYGSGPGVLGPYGPGQSGSGVLGPGVLGPYASAAAAAGPGVLGPYGPGSSAAAAAGQYGYGPGVLGPYGPGASGQNGPGSGQYGPGVLGPGQSAAAAAGPGVLGPYGPGASAAAAAGQYGPGVLGPGQYGPGSSGPGVLGPYGPGSSAAAAAGQYGPGVLGPYGPGQSAAAAAGQYVLGPGVLGPYGPGASGPGVLGPYGPGASAAAAAGPGQYGPGVLGPSASAAAAAGQYGSGPGQYGPYGPGQSGPGSGVLGQGPYGPGASAAAAAGQYGPGVLGPYGPGQSAAAAAGPGSGQYGPGASGQNGPGSGQYGPGVLGPGQSAAAAAGQYVLGPGVLGPYGPGASAAAAAGQYGSGPGVLGPYGPGQSGSGVLGPGVLGPYASAAAAAGPGSGVLGPGAS

SEQ ID NO: 26 - protein, 590 aa, Artificial Sequence
  Met-PRT965
MGPGTSGPYGPGASAAAAAGANGPGSGTSGPGASGAYGPGTSGPGTSGPGSSAAAAAGPGAYGPGTSGPSASAAAAAGPGSGTSGPGASGAYGPGTSGPGTSGPGSSAAAAAGAYGSGPGTSGPYGSAAAAAGPGSGAYGAGPYGPGASGPGAYGPGTSGPSASAAAAAGSGTSGPGAYGPYASAAAAAGAYGSGPGTSGPYGPGASGSGTSGPGTSGPYASAAAAAGPGTSGPYGPGSSAAAAAGAYGYGPGTSGPYGPGASGANGPGSGAYGPGTSGPGASAAAAAGPGTSGPYGPGASAAAAAGAYGPGTSGPGAYGPGSSGPGTSGPYGPGSSAAAAAGAYGPGTSGPYGPGASAAAAAGAYTSGPGTSGPYGPGASGPGTSGPYGPGASAAAAAGPGAYGPGTSGPSASAAAAAGAYGSGPGAYGPYGPGASGPGSGTSGAGPYGPGASAAAAAGAYGPGTSGPYGPGASAAAAAGPGSGAYGPGASGANGPGSGAYGPGTSGPGASAAAAAGAYTSGPGTSGPYGPGASAAAAAGAYGSGPGTSGPYGPGASGSGTSGPGTSGPYASAAAAAGPGSGTSGPGAS

SEQ ID NO: 27 - protein, 593 aa, Artificial Sequence
  Met-PRT889
MGSSGPGVLGPYGPGASAAAAAGINGPGSGVLGPGISGIYGPGVLGPGVLGPGSSAAAAAGPGIYGPGVLGPSASAAAAAGPGSGVLGPGASGIYGPGVLGPGVLGPGSSAAAAAGIYGSGPGVLGPYGSAAAAAGPGSGIYGIGPYGPGASGPGIYGPGVLGPSASAAAAAGSGVLGPGIYGPYASAAAAAGIYGSGPGVLGPYGPGISGSGVLGPGVLGPYASAAAAAGPGVLGPYGPGSSAAAAAGIYGYGPGVLGPYGPGASGINGPGSGIYGPGVLGPGISAAAAAGPGVLGPYGPGASAAAAAGIYGPGVLGPGIYGPGSSGPGVLGPYGPGSSAAAAAGIYGPGVLGPYGPGISAAAAAGIYVLGPGVLGPYGPGASGPGVLGPYGPGASAAAAAGPGIYGPGVLGPSASAAAAAGIYGSGPGIYGPYGPGISGPGSGVLGIGPYGPGASAAAAAGIYGPGVLGPYGPGISAAAAAGPGSGIYGPGASGINGPGSGIYGPGVLGPGISAAAAAGIYVLGPGVLGPYGPGASAAAAAGIYGSGPGVLGPYGPGISGSGVLGPGVLGPYASAAAAAGPGSGVLGPGAS

SEQ ID NO: 28 - protein, 590 aa, Artificial Sequence
  Met-PRT916
MGPGVIGPYGPGASAAAAAGLNGPGSGVIGPGLSGLYGPGVIGPGVIGPGSSAAAAAGPGLYGPGVIGPSASAAAAAGPGSGVIGPGASGLYGPGVIGPGVIGPGSSAAAAAGLYGSGPGVIGPYGSAAAAAGPGSGLYGLGPYGPGASGPGLYGPGVIGPSASAAAAAGSGVIGPGLYGPYASAAAAAGLYGSGPGVIGPYGPGLSGSGVIGPGVIGPYASAAAAAGPGVIGPYGPGSSAAAAAGLYGYGPGVIGPYGPGASGLNGPGSGLYGPGVIGPGLSAAAAAGPGVIGPYGPGASAAAAAGLYGPGVIGPGLYGPGSSGPGVIGPYGPGSSAAAAAGLYGPGVIGPYGPGLSAAAAAGLYVIGPGVIGPYGPGASGPGVIGPYGPGASAAAAAGPGLYGPGVIGPSASAAAAAGLYGSGPGLYGPYGPGLSGPGSGVIGLGPYGPGASAAAAAGLYGPGVIGPYGPGLSAAAAAGPGSGLYGPGASGLNGPGSGLYGPGVIGPGLSAAAAAGLYVIGPGVIGPYGPGASAAAAAGLYGSGPGVIGPYGPGLSGSGVIGPGVIGPYASAAAAAGPGSGVIGPGAS

SEQ ID NO: 29 - protein, 590 aa, Artificial Sequence
  Met-PRT918
MGPGVFGPYGPGASAAAAAGINGPGSGVFGPGISGIYGPGVFGPGVFGPGSSAAAAAGPGIYGPGVFGPSASAAAAAGPGSGVFGPGASGIYGPGVFGPGVFGPGSSAAAAAGIYGSGPGVFGPYGSAAAAAGPGSGIYGIGPYGPGASGPGIYGPGVFGPSASAAAAAGSGVFGPGIYGPYASAAAAAGIYGSGPGVFGPYGPGISGSGVFGPGVFGPYASAAAAAGPGVFGPYGPGSSAAAAAGIYGYGPGVFGPYGPGASGINGPGSGIYGPGVFGPGISAAAAAGPGVFGPYGPGASAAAAAGIYGPGVFGPGIYGPGSSGPGVFGPYGPGSSAAAAAGIYGPGVFGPYGPGISAAAAAGIYVFGPGVFGPYGPGASGPGVFGPYGPGASAAAAAGPGIYGPGVFGPSASAAAAAGIYGSGPGIYGPYGPGISGPGSGVFGIGPYGPGASAAAAAGIYGPGVFGPYGPGISAAAAAGPGSGIYGPGASGINGPGSGIYGPGVFGPGISAAAAAGIYVFGPGVFGPYGPGASAAAAAGIYGSGPGVFGPYGPGISGSGVFGPGVFGPYASAAAAAGPGSGVFGPGAS

SEQ ID NO: 30 - protein, 565 aa, Artificial Sequence
  Met-PRT699
MGPGVLGPYGPGASAAAAAAAGSNGPGSGVLGPGQSGQYGPGVLGPGVLGPGSSAAAAAAAGPGQYGPGVLGPSASAAAAAAAGPGSGVLGPGASGQYGPGVLGPGVLGPGSSAAAAAAAGSYGSGPGVLGPYGSAAAAAAAGPGSGQYGQGPYGPGASGPGQYGPGVLGPSASAAAAAAAGSGVLGPGQYGPYASAAAAAAAGSYGSGPGVLGPYGPGQSGSGVLGPGVLGPYASAAAAAAAGPGVLGPYGPGSSAAAAAAAGSYGYGPGVLGPYGPGASGQNGPGSGQYGPGVLGPGPSAAAAAAAGPGVLGPYGPGASAAAAAAAGSYGPGVLGPGQYGPGSSGPGVLGPYGPGSSAAAAAAAGSYGPGVLGPYGPGPSAAAAAAAGSYVLGPGVLGPYGPGASGPGVLGPYGPGASAAAAAAAGPGQYGPGVLGPSASAAAAAAAGSYGSGPGQYGPYGPGQSGPGSGVLGQGPYGPGASAAAAAAAGSYGPGVLGPYGPGPSAAAAAAAGPGSGQYGPGASGQNGPGSGQYGPGVLGPGPSAAAAAAAGPGSGVLGPGAS

SEQ ID NO: 31 - protein, 565 aa, Artificial Sequence
  Met-PRT698
MGPGVLGPYGPGASAAAAAAAGSNGPGSGVLGPGISGIYGPGVLGPGVLGPGSSAAAAAAAGPGIYGPGVLGPSASAAAAAAAGPGSGVLGPGASGIYGPGVLGPGVLGPGSSAAAAAAAGSYGSGPGVLGPYGSAAAAAAAGPGSGIYGIGPYGPGASGPGIYGPGVLGPSASAAAAAAAGSGVLGPGIYGPYASAAAAAAAGSYGSGPGVLGPYGPGISGSGVLGPGVLGPYASAAAAAAAGPGVLGPYGPGSSAAAAAAAGSYGYGPGVLGPYGPGASGINGPGSGIYGPGVLGPGPSAAAAAAAGPGVLGPYGPGASAAAAAAAGSYGPGVLGPGIYGPGSSGPGVLGPYGPGSSAAAAAAAGSYGPGVLGPYGPGPSAAAAAAAGSYVLGPGVLGPYGPGASGPGVLGPYGPGASAAAAAAAGPGIYGPGVLGPSASAAAAAAAGSYGSGPGIYGPYGPGISGPGSGVLGIGPYGPGASAAAAAAAGSYGPGVLGPYGPGPSAAAAAAAGPGSGIYGPGASGINGPGSGIYGPGVLGPGPSAAAAAAAGPGSGVLGPGAS

SEQ ID NO: 32 - protein, 1179 aa, Artificial Sequence
  Met-PRT966
MGPGVFGPYGPGASAAAAAGINGPGSGVFGPGISGIYGPGVFGPGVFGPGSSAAAAAGPGIYGPGVFGPSASAAAAAGPGSGVFGPGASGIYGPGVFGPGVFGPGSSAAAAAGIYGSGPGVFGPYGSAAAAAGPGSGIYGIGPYGPGASGPGIYGPGVFGPSASAAAAAGSGVFGPGIYGPYASAAAAAGIYGSGPGVFGPYGPGISGSGVFGPGVFGPYASAAAAAGPGVFGPYGPGSSAAAAAGIYGYGPGVFGPYGPGASGINGPGSGIYGPGVFGPGISAAAAAGPGVFGPYGPGASAAAAAGIYGPGVFGPGIYGPGSSGPGVFGPYGPGSSAAAAAGIYGPGVFGPYGPGISAAAAAGIYVFGPGVFGPYGPGASGPGVFGPYGPGASAAAAAGPGIYGPGVFGPSASAAAAAGIYGSGPGIYGPYGPGISGPGSGVFGIGPYGPGASAAAAAGIYGPGVFGPYGPGISAAAAAGPGSGIYGPGASGINGPGSGIYGPGVFGPGISAAAAAGIYVFGPGVFGPYGPGASAAAAAGIYGSGPGVFGPYGPGISGSGVFGPGVFGPYASAAAAAGPGSGVFGPGASGPGVFGPYGPGASAAAAAGINGPGSGVFGPGISGIYGPGVFGPGVFGPGSSAAAAAGPGIYGPGVFGPSASAAAAAGPGSGVFGPGASGIYGPGVFGPGVFGPGSSAAAAAGIYGSGPGVFGPYGSAAAAAGPGSGIYGIGPYGPGASGPGIYGPGVFGPSASAAAAAGSGVFGPGIYGPYASAAAAAGIYGSGPGVFGPYGPGISGSGVFGPGVFGPYASAAAAAGPGVFGPYGPGSSAAAAAGIYGYGPGVFGPYGPGASGINGPGSGIYGPGVFGPGISAAAAAGPGVFGPYGPGASAAAAAGIYGPGVFGPGIYGPGSSGPGVFGPYGPGSSAAAAAGIYGPGVFGPYGPGISAAAAAGIYVFGPGVFGPYGPGASGPGVFGPYGPGASAAAAAGPGIYGPGVFGPSASAAAAAGIYGSGPGIYGPYGPGISGPGSGVFGIGPYGPGASAAAAAGIYGPGVFGPYGPGISAAAAAGPGSGIYGPGASGINGPGSGIYGPGVFGPGISAAAAAGIYVFGPGVFGPYGPGASAAAAAGIYGSGPGVFGPYGPGISGSGVFGPGVFGPYASAAAAAGPGSGVFGPGAS

SEQ ID NO: 33 - protein, 601 aa, Artificial Sequence
  PRT888
MHHHHHHSSGSSGPGVLGPYGPGASAAAAAGQNGPGSGVLGPGQSGQYGPGVLGPGVLGPGSSAAAAAGPGQYGPGVLGPSASAAAAAGPGSGVLGPGASGQYGPGVLGPGVLGPGSSAAAAAGQYGSGPGVLGPYGSAAAAAGPGSGQYGQGPYGPGASGPGQYGPGVLGPSASAAAAAGSGVLGPGQYGPYASAAAAAGQYGSGPGVLGPYGPGQSGSGVLGPGVLGPYASAAAAAGPGVLGPYGPGSSAAAAAGQYGYGPGVLGPYGPGASGQNGPGSGQYGPGVLGPGQSAAAAAGPGVLGPYGPGASAAAAAGQYGPGVLGPGQYGPGSSGPGVLGPYGPGSSAAAAAGQYGPGVLGPYGPGQSAAAAAGQYVLGPGVLGPYGPGASGPGVLGPYGPGASAAAAAGPGQYGPGVLGPSASAAAAAGQYGSGPGQYGPYGPGQSGPGSGVLGQGPYGPGASAAAAAGQYGPGVLGPYGPGQSAAAAAGPGSGQYGPGASGQNGPGSGQYGPGVLGPGQSAAAAAGQYVLGPGVLGPYGPGASAAAAAGQYGSGPGVLGPYGPGQSGSGVLGPGVLGPYASAAAAAGPGSGVLGPGAS

SEQ ID NO: 34 - protein, 601 aa, Artificial Sequence
  PRT965
MHHHHHHSSGSSGPGTSGPYGPGASAAAAAGANGPGSGTSGPGASGAYGPGTSGPGTSGPGSSAAAAAGPGAYGPGTSGPSASAAAAAGPGSGTSGPGASGAYGPGTSGPGTSGPGSSAAAAAGAYGSGPGTSGPYGSAAAAAGPGSGAYGAGPYGPGASGPGAYGPGTSGPSASAAAAAGSGTSGPGAYGPYASAAAAAGAYGSGPGTSGPYGPGASGSGTSGPGTSGPYASAAAAAGPGTSGPYGPGSSAAAAAGAYGYGPGTSGPYGPGASGANGPGSGAYGPGTSGPGASAAAAAGPGTSGPYGPGASAAAAAGAYGPGTSGPGAYGPGSSGPGTSGPYGPGSSAAAAAGAYGPGTSGPYGPGASAAAAAGAYTSGPGTSGPYGPGASGPGTSGPYGPGASAAAAAGPGAYGPGTSGPSASAAAAAGAYGSGPGAYGPYGPGASGPGSGTSGAGPYGPGASAAAAAGAYGPGTSGPYGPGASAAAAAGPGSGAYGPGASGANGPGSGAYGPGTSGPGASAAAAAGAYTSGPGTSGPYGPGASAAAAAGAYGSGPGTSGPYGPGASGSGTSGPGTSGPYASAAAAAGPGSGTSGPGAS

SEQ ID NO: 35 - protein, 601 aa, Artificial Sequence
  PRT889
MHHHHHHSSGSSGPGVLGPYGPGASAAAAAGINGPGSGVLGPGISGIYGPGVLGPGVLGPGSSAAAAAGPGIYGPGVLGPSASAAAAAGPGSGVLGPGASGIYGPGVLGPGVLGPGSSAAAAAGIYGSGPGVLGPYGSAAAAAGPGSGIYGIGPYGPGASGPGIYGPGVLGPSASAAAAAGSGVLGPGIYGPYASAAAAAGIYGSGPGVLGPYGPGISGSGVLGPGVLGPYASAAAAAGPGVLGPYGPGSSAAAAAGIYGYGPGVLGPYGPGASGINGPGSGIYGPGVLGPGISAAAAAGPGVLGPYGPGASAAAAAGIYGPGVLGPGIYGPGSSGPGVLGPYGPGSSAAAAAGIYGPGVLGPYGPGISAAAAAGIYVLGPGVLGPYGPGASGPGVLGPYGPGASAAAAAGPGIYGPGVLGPSASAAAAAGIYGSGPGIYGPYGPGISGPGSGVLGIGPYGPGASAAAAAGIYGPGVLGPYGPGISAAAAAGPGSGIYGPGASGINGPGSGIYGPGVLGPGISAAAAAGIYVLGPGVLGPYGPGASAAAAAGIYGSGPGVLGPYGPGISGSGVLGPGVLGPYASAAAAAGPGSGVLGPGAS

SEQ ID NO: 36 - protein, 601 aa, Artificial Sequence
  PRT916
MHHHHHHSSGSSGPGVIGPYGPGASAAAAAGLNGPGSGVIGPGLSGLYGPGVIGPGVIGPGSSAAAAAGPGLYGPGVIGPSASAAAAAGPGSGVIGPGASGLYGPGVIGPGVIGPGSSAAAAAGLYGSGPGVIGPYGSAAAAAGPGSGLYGLGPYGPGASGPGLYGPGVIGPSASAAAAAGSGVIGPGLYGPYASAAAAAGLYGSGPGVIGPYGPGLSGSGVIGPGVIGPYASAAAAAGPGVIGPYGPGSSAAAAAGLYGYGPGVIGPYGPGASGLNGPGSGLYGPGVIGPGLSAAAAAGPGVIGPYGPGASAAAAAGLYGPGVIGPGLYGPGSSGPGVIGPYGPGSSAAAAAGLYGPGVIGPYGPGLSAAAAAGLYVIGPGVIGPYGPGASGPGVIGPYGPGASAAAAAGPGLYGPGVIGPSASAAAAAGLYGSGPGLYGPYGPGLSGPGSGVIGLGPYGPGASAAAAAGLYGPGVIGPYGPGLSAAAAAGPGSGLYGPGASGLNGPGSGLYGPGVIGPGLSAAAAAGLYVIGPGVIGPYGPGASAAAAAGLYGSGPGVIGPYGPGLSGSGVIGPGVIGPYASAAAAAGPGSGVIGPGAS

SEQ ID NO: 37 - protein, 601 aa, Artificial Sequence
  PRT918
MHHHHHHSSGSSGPGVFGPYGPGASAAAAAGINGPGSGVFGPGISGIYGPGVFGPGVFGPGSSAAAAAGPGIYGPGVFGPSASAAAAAGPGSGVFGPGASGIYGPGVFGPGVFGPGSSAAAAAGIYGSGPGVFGPYGSAAAAAGPGSGIYGIGPYGPGASGPGIYGPGVFGPSASAAAAAGSGVFGPGIYGPYASAAAAAGIYGSGPGVFGPYGPGISGSGVFGPGVFGPYASAAAAAGPGVFGPYGPGSSAAAAAGIYGYGPGVFGPYGPGASGINGPGSGIYGPGVFGPGISAAAAAGPGVFGPYGPGASAAAAAGIYGPGVFGPGIYGPGSSGPGVFGPYGPGSSAAAAAGIYGPGVFGPYGPGISAAAAAGIYVFGPGVFGPYGPGASGPGVFGPYGPGASAAAAAGPGIYGPGVFGPSASAAAAAGIYGSGPGIYGPYGPGISGPGSGVFGIGPYGPGASAAAAAGIYGPGVFGPYGPGISAAAAAGPGSGIYGPGASGINGPGSGIYGPGVFGPGISAAAAAGIYVFGPGVFGPYGPGASAAAAAGIYGSGPGVFGPYGPGISGSGVFGPGVFGPYASAAAAAGPGSGVFGPGAS

SEQ ID NO: 38 - protein, 576 aa, Artificial Sequence
  PRT699
MHHHHHHSSGSSGPGVLGPYGPGASAAAAAAAGSNGPGSGVLGPGQSGQYGPGVLGPGVLGPGSSAAAAAAAGPGQYGPGVLGPSASAAAAAAAGPGSGVLGPGASGQYGPGVLGPGVLGPGSSAAAAAAAGSYGSGPGVLGPYGSAAAAAAAGPGSGQYGQGPYGPGASGPGQYGPGVLGPSASAAAAAAAGSGVLGPGQYGPYASAAAAAAAGSYGSGPGVLGPYGPGQSGSGVLGPGVLGPYASAAAAAAAGPGVLGPYGPGSSAAAAAAAGSYGYGPGVLGPYGPGASGQNGPGSGQYGPGVLGPGPSAAAAAAAGPGVLGPYGPGASAAAAAAAGSYGPGVLGPGQYGPGSSGPGVLGPYGPGSSAAAAAAAGSYGPGVLGPYGPGPSAAAAAAAGSYVLGPGVLGPYGPGASGPGVLGPYGPGASAAAAAAAGPGQYGPGVLGPSASAAAAAAAGSYGSGPGQYGPYGPGQSGPGSGVLGQGPYGPGASAAAAAAAGSYGPGVLGPYGPGPSAAAAAAAGPGSGQYGPGASGQNGPGSGQYGPGVLGPGPSAAAAAAAGPGSGVLGPGAS

SEQ ID NO: 39 - protein, 576 aa, Artificial Sequence
  PRT698
MHHHHHHSSGSSGPGVLGPYGPGASAAAAAAAGSNGPGSGVLGPGISGIYGPGVLGPGVLGPGSSAAAAAAAGPGIYGPGVLGPSASAAAAAAAGPGSGVLGPGASGIYGPGVLGPGVLGPGSSAAAAAAAGSYGSGPGVLGPYGSAAAAAAAGPGSGIYGIGPYGPGASGPGIYGPGVLGPSASAAAAAAAGSGVLGPGIYGPYASAAAAAAAGSYGSGPGVLGPYGPGISGSGVLGPGVLGPYASAAAAAAAGPGVLGPYGPGSSAAAAAAAGSYGYGPGVLGPYGPGASGINGPGSGIYGPGVLGPGPSAAAAAAAGPGVLGPYGPGASAAAAAAAGSYGPGVLGPGIYGPGSSGPGVLGPYGPGSSAAAAAAAGSYGPGVLGPYGPGPSAAAAAAAGSYVLGPGVLGPYGPGASGPGVLGPYGPGASAAAAAAAGPGIYGPGVLGPSASAAAAAAAGSYGSGPGIYGPYGPGISGPGSGVLGIGPYGPGASAAAAAAAGSYGPGVLGPYGPGPSAAAAAAAGPGSGIYGPGASGINGPGSGIYGPGVLGPGPSAAAAAAAGPGSGVLGPGAS

SEQ ID NO: 40 - protein, 1190 aa, Artificial Sequence
  PRT966
MHHHHHHSSGSSGPGVFGPYGPGASAAAAAGINGPGSGVFGPGISGIYGPGVFGPGVFGPGSSAAAAAGPGIYGPGVFGPSASAAAAAGPGSGVFGPGASGIYGPGVFGPGVFGPGSSAAAAAGIYGSGPGVFGPYGSAAAAAGPGSGIYGIGPYGPGASGPGIYGPGVFGPSASAAAAAGSGVFGPGIYGPYASAAAAAGIYGSGPGVFGPYGPGISGSGVFGPGVFGPYASAAAAAGPGVFGPYGPGSSAAAAAGIYGYGPGVFGPYGPGASGINGPGSGIYGPGVFGPGISAAAAAGPGVFGPYGPGASAAAAAGIYGPGVFGPGIYGPGSSGPGVFGPYGPGSSAAAAAGIYGPGVFGPYGPGISAAAAAGIYVFGPGVFGPYGPGASGPGVFGPYGPGASAAAAAGPGIYGPGVFGPSASAAAAAGIYGSGPGIYGPYGPGISGPGSGVFGIGPYGPGASAAAAAGIYGPGVFGPYGPGISAAAAAGPGSGIYGPGASGINGPGSGIYGPGVFGPGISAAAAAGIYVFGPGVFGPYGPGASAAAAAGIYGSGPGVFGPYGPGISGSGVFGPGVFGPYASAAAAAGPGSGVFGPGASGPGVFGPYGPGASAAAAAGINGPGSGVFGPGISGIYGPGVFGPGVFGPGSSAAAAAGPGIYGPGVFGPSASAAAAAGPGSGVFGPGASGIYGPGVFGPGVFGPGSSAAAAAGIYGSGPGVFGPYGSAAAAAGPGSGIYGIGPYGPGASGPGIYGPGVFGPSASAAAAAGSGVFGPGIYGPYASAAAAAGIYGSGPGVFGPYGPGISGSGVFGPGVFGPYASAAAAAGPGVFGPYGPGSSAAAAAGIYGYGPGVFGPYGPGASGINGPGSGIYGPGVFGPGISAAAAAGPGVFGPYGPGASAAAAAGIYGPGVFGPGIYGPGSSGPGVFGPYGPGSSAAAAAGIYGPGVFGPYGPGISAAAAAGIYVFGPGVFGPYGPGASGPGVFGPYGPGASAAAAAGPGIYGPGVFGPSASAAAAAGIYGSGPGIYGPYGPGISGPGSGVFGIGPYGPGASAAAAAGIYGPGVFGPYGPGISAAAAAGPGSGIYGPGASGINGPGSGIYGPGVFGPGISAAAAAGIYVFGPGVFGPYGPGASAAAAAGIYGSGPGVFGPYGPGISGSGVFGPGVFGPYASAAAAAGPGSGVFGPGAS

SEQ ID NO: 41 - protein, 590 aa, Artificial Sequence
  Met-PRT917
MGPGLIGPYGPGASAAAAAGVNGPGSGLIGPGVSGVYGPGLIGPGLIGPGSSAAAAAGPGVYGPGLIGPSASAAAAAGPGSGLIGPGASGVYGPGLIGPGLIGPGSSAAAAAGVYGSGPGLIGPYGSAAAAAGPGSGVYGVGPYGPGASGPGVYGPGLIGPSASAAAAAGSGLIGPGVYGPYASAAAAAGVYGSGPGLIGPYGPGVSGSGLIGPGLIGPYASAAAAAGPGLIGPYGPGSSAAAAAGVYGYGPGLIGPYGPGASGVNGPGSGVYGPGLIGPGVSAAAAAGPGLIGPYGPGASAAAAAGVYGPGLIGPGVYGPGSSGPGLIGPYGPGSSAAAAAGVYGPGLIGPYGPGVSAAAAAGVYLIGPGLIGPYGPGASGPGLIGPYGPGASAAAAAGPGVYGPGLIGPSASAAAAAGVYGSGPGVYGPYGPGVSGPGSGLIGVGPYGPGASAAAAAGVYGPGLIGPYGPGVSAAAAAGPGSGVYGPGASGVNGPGSGVYGPGLIGPGVSAAAAAGVYLIGPGLIGPYGPGASAAAAAGVYGSGPGLIGPYGPGVSGSGLIGPGLIGPYASAAAAAGPGSGLIGPGAS

SEQ ID NO: 42 - protein, 587 aa, Artificial Sequence
  Met-PRT1028
MGPGIFGPYGPGASAAAAAGTGPGSGIFGPGTSGTYGPGIFGPGIFGPGSSAAAAAGPGTYGPGIFGPSASAAAAAGPGSGIFGPGASGTYGPGIFGPGIFGPGSSAAAAAGTYGSGPGIFGPYGSAAAAAGPGSGTYGTGPYGPGASGPGTYGPGIFGPSASAAAAAGSGIFGPGTYGPYASAAAAAGTYGSGPGIFGPYGPGTSGSGIFGPGIFGPYASAAAAAGPGIFGPYGPGSSAAAAAGTYGYGPGIFGPYGPGASGTGPGSGTYGPGIFGPGTSAAAAAGPGIFGPYGPGASAAAAAGTYGPGIFGPGTYGPGSSGPGIFGPYGPGSSAAAAAGTYGPGIFGPYGPGTSAAAAAGTYIFGPGIFGPYGPGASGPGIFGPYGPGASAAAAAGPGTYGPGIFGPSASAAAAAGTYGSGPGTYGPYGPGTSGPGSGIFGTGPYGPGASAAAAAGTYGPGIFGPYGPGTSAAAAAGPGSGTYGPGASGTGPGSGTYGPGIFGPGTSAAAAAGTYIFGPGIFGPYGPGASAAAAAGTYGSGPGIFGPYGPGTSGSGIFGPGIFGPYASAAAAAGPGSGIFGPGAS

SEQ ID NO: 43 - protein, 601 aa, Artificial Sequence
  PRT917
MHHHHHHSSGSSGPGLIGPYGPGASAAAAAGVNGPGSGLIGPGVSGVYGPGLIGPGLIGPGSSAAAAAGPGVYGPGLIGPSASAAAAAGPGSGLIGPGASGVYGPGLIGPGLIGPGSSAAAAAGVYGSGPGLIGPYGSAAAAAGPGSGVYGVGPYGPGASGPGVYGPGLIGPSASAAAAAGSGLIGPGVYGPYASAAAAAGVYGSGPGLIGPYGPGVSGSGLIGPGLIGPYASAAAAAGPGLIGPYGPGSSAAAAAGVYGYGPGLIGPYGPGASGVNGPGSGVYGPGLIGPGVSAAAAAGPGLIGPYGPGASAAAAAGVYGPGLIGPGVYGPGSSGPGLIGPYGPGSSAAAAAGVYGPGLIGPYGPGVSAAAAAGVYLIGPGLIGPYGPGASGPGLIGPYGPGASAAAAAGPGVYGPGLIGPSASAAAAAGVYGSGPGVYGPYGPGVSGPGSGLIGVGPYGPGASAAAAAGVYGPGLIGPYGPGVSAAAAAGPGSGVYGPGASGVNGPGSGVYGPGLIGPGVSAAAAAGVYLIGPGLIGPYGPGASAAAAAGVYGSGPGLIGPYGPGVSGSGLIGPGLIGPYASAAAAAGPGSGLIGPGAS

SEQ ID NO: 44 - protein, 598 aa, Artificial Sequence
  PRT1028
MHHHHHHSSGSSGPGIFGPYGPGASAAAAAGTGPGSGIFGPGTSGTYGPGIFGPGIFGPGSSAAAAAGPGTYGPGIFGPSASAAAAAGPGSGIFGPGASGTYGPGIFGPGIFGPGSSAAAAAGTYGSGPGIFGPYGSAAAAAGPGSGTYGTGPYGPGASGPGTYGPGIFGPSASAAAAAGSGIFGPGTYGPYASAAAAAGTYGSGPGIFGPYGPGTSGSGIFGPGIFGPYASAAAAAGPGIFGPYGPGSSAAAAAGTYGYGPGIFGPYGPGASGTGPGSGTYGPGIFGPGTSAAAAAGPGIFGPYGPGASAAAAAGTYGPGIFGPGTYGPGSSGPGIFGPYGPGSSAAAAAGTYGPGIFGPYGPGTSAAAAAGTYIFGPGIFGPYGPGASGPGIFGPYGPGASAAAAAGPGTYGPGIFGPSASAAAAAGTYGSGPGTYGPYGPGTSGPGSGIFGTGPYGPGASAAAAAGTYGPGIFGPYGPGTSAAAAAGPGSGTYGPGASGTGPGSGTYGPGIFGPGTSAAAAAGTYIFGPGIFGPYGPGASAAAAAGTYGSGPGIFGPYGPGTSGSGIFGPGIFGPYASAAAAAGPGSGIFGPGAS

(19) United States
(12) Patent Application Publication
(10) Pub. No.: US 2021/0214404 A1
(43) Pub. Date: Jul. 15, 2021

(54) Artificial Hair Fiber, Method for Manufacturing Same, and Artificial Hair

(71) Applicants: Spiber Inc., Tsuruoka-shi, Yamagata (JP); Kojima Industries Corporation, Aichi (JP)

(72) Inventors: Masato Matsuo, Tsuruoka-shi, Yamagata (JP); Hideto Ishii, Tsuruoka-shi, Yamagata (JP); Yunosuke Abe, Tsuruoka-shi, Yamagata (JP)

(73) Assignees: Spiber Inc., Tsuruoka-shi, Yamagata (JP); Kojima Industries Corporation, Aichi (JP)

(21) Appl. No.: 17/262,350

(22) PCT Filed: Jul. 24, 2019
(86) PCT No.: PCT/JP2019/029077

§ 371 Date: Jan. 22, 2021

(30) Foreign Application Priority Data
Jul. 25, 2018 (JP) 2018-139563

(51) Int. Cl. C07K 14/435; A41G 3/00

(52) U.S. Cl. CPC C07K 14/43518; A41G 3/0083

(57) ABSTRACT

The present invention pertains to an artificial hair fiber that includes an artificial fibroin fiber containing a modified fibroin and that expands when being in a wet state and shrinks when being dried after the wet state.

### TECHNICAL FIELD

The present invention relates to artificial hair fiber, a method for manufacturing the same, and artificial hair.

### BACKGROUND ART

In addition to human hair, artificial hair made of synthetic fibers or the like is used as a material for wigs, hair attachments, or the like (for example, Patent Literature 1).

### CITATION LIST

### Patent Literature

[Patent Literature 1] Japanese Unexamined Patent Publication No. 2007-297737

### SUMMARY OF INVENTION

### Technical Problem

Human hair is preferably used in terms of texture and the like. However, since the hair quality (a thickness, a hardness, and the like) of human hair varies greatly according to race, gender, individual differences, and the like, it is difficult to stably obtain uniform quality hair. In addition, in recent years, as the number of human hair sellers has decreased, it has become difficult to obtain a large amount of human hair.

On the other hand, artificial hair fibers made of synthetic fibers such as polyester fibers and acrylic fibers and having a desired length can be stably obtained in a large amount with uniform quality. However, synthetic fibers have a significantly different texture as compared with human hair, and also have a large variation in characteristics. For example, human hair has a property in which it elongates to a predetermined length when it absorbs water and then returns to its original length when it dries, whereas synthetic fibers do not have such a property. Therefore, when artificial hair fibers made of synthetic fibers are used, it is inevitable that a feeling of strangeness compared with human hair will occur.

An object of the present invention is to provide an artificial hair fibers capable of being stably supplied and with which occurrence of a feeling of strangeness in comparison with human hair is curbed.

### Solution to Problem

In the process of various studies by the inventors, it has been found that an artificial fibroin fiber including a modified fibroin expands and contracts in the same manner as human hair when it is wet and dried. Then, as a result of repeated diligent research based on such findings, the present invention has been completed.

The present invention relates to, for example, the following inventions.

[1] There is provided an artificial hair fiber including an artificial fibroin fiber containing a modified fibroin, wherein the artificial hair fiber expands when being in a wet state and contracts when being dried from the wet state.

[2] In the artificial hair fiber described in [1], a restoration rate defined by the following Formula (1) may be 95% or more.

Restoration rate=(length of artificial fibroin fiber when dried from wet state/length of artificial fibroin fiber before wet state)×100(%) (1)

[3] In the artificial hair fiber described in [1] or [2], the artificial fibroin fiber may be a fiber having a contraction history in which the fiber is irreversibly contracted by contact with water after spinning, and a contraction rate A defined by the following Formula (2) may be 2% or more.

Contraction rate A={1−(length of fiber irreversibly contracted by contact with water after spinning/length of fiber after spinning and before contact with water)}×100(%) (2)

[4] In the artificial hair fiber described in any one of [1] to [3], the artificial fibroin fiber may be a fiber having a contraction history in which the fiber is irreversibly contracted by contact with water after spinning and then further contracted by drying, and a contraction rate B defined by the following Formula (3) may be more than 7%.

Contraction rate B={1−(length of fiber irreversibly contracted by contact with water after spinning and then further contracted by drying/length of fiber after spinning and before contact with water)}×100(%) (3)

[5] In the artificial hair fiber described in any one of [1] to [4], the modified fibroin may be a modified spider silk fibroin.

[6] In the artificial hair fiber described in any one of [1] to [5], a recess which extends in a fiber axis direction may be provided in a surface.

[7] In the artificial hair fiber described in any one of [1] to [6], an expansion rate defined by the following Formula (4) may be 17% or less.

Expansion rate={(length of artificial fibroin fiber in wet state/length of artificial fibroin fiber before wet state)−1}×100(%) (4)

[8] In the artificial hair fiber described in any one of [1] to [7], a contraction rate C defined by the following Formula (5) may be 17% or less.

Contraction rate C={1−(length of artificial fibroin fiber when dried from wet state/length of artificial fibroin fiber in wet state)×100(%) (5)

[9] In the artificial hair fiber described in any one of [1] to [8], a heat contraction rate defined by the following Formula (6) may be 4% or less.

Heat contraction rate={1−(length of artificial fibroin fiber when heated to 160° C./length of artificial fibroin fiber before heating)}×100(%) (6)

[10] In the artificial hair fiber described in any one of [1] to [9], the artificial hair fiber may contain substantially no residual stress generated by drawing in a spinning process.

[11] There is provided a method for manufacturing an artificial hair fiber, the method including a contraction step in which a raw material fiber after spinning and before contact with water is brought into contact with water to be irreversibly contracted, then dried and further contracted, wherein the raw material fiber contains a modified fibroin.

[12] In the method described in [11], the raw material fiber may be a fiber having a contraction rate A of 2% or more defined by the following Formula (2).

Contraction rate A={1−(length of fiber irreversibly contracted by contact with water after spinning/length of fiber after spinning and before contact with water)}×100(%) (2)

[13] In the method described in [11] or [12], the raw material fiber may be a fiber having a contraction rate B of more than 7% defined by the following Formula (3).

Contraction rate B={1−(length of fiber irreversibly contracted by contact with water after spinning and then further contracted by drying/length of fiber after spinning and before contact with water)}×100(%) (3)

[14] In the method described in any one of [11] to [13], in the contraction step, substantially all residual stress in the raw material fiber generated by drawing in a spinning process may be released.

[15] In the method described in any one of [11] to [14], the contraction step may be performed without relaxing the raw material fiber.

[16] In the method described in any one of [11] to [15], the raw material fiber may be formed by introducing a spinning dope solution containing the modified fibroin and a solvent into a coagulating solution, removing the solvent from the spinning dope solution and coagulating the spinning dope solution.

[17] In the method described in any one of [11] to [16], the modified fibroin may be a modified spider silk fibroin.

[18] There is provided an artificial hair including the artificial hair fiber described in any one of [1] to [10].

### Advantageous Effects of Invention

According to the present invention, it is possible to provide an artificial hair fiber which can be stably supplied and can curb occurrence of a feeling of strangeness with human hair.

### BRIEF DESCRIPTION OF DRAWINGS

FIG. 1 is a schematic diagram showing an example of a domain sequence of a modified fibroin.

FIG. 2 is a diagram showing a distribution of values of z/w (%) in a naturally occurring fibroin.

FIG. 3 is a diagram showing a distribution of values of x/y (%) in the naturally occurring fibroin.

FIG. 4 is a schematic diagram showing an example of the domain sequence of the modified fibroin.

FIG. 5 is a schematic diagram showing an example of the domain sequence of the modified fibroin.

FIG. 6 is an explanatory diagram schematically showing an example of a spinning apparatus for producing raw material fibers.

FIG. 7 is a diagram showing an example of a change in a length of the raw material fiber (fiber including the modified fibroin) due to contact with water.

FIG. 8 is an explanatory diagram schematically showing an example of a manufacturing apparatus for manufacturing artificial hair fibers (artificial fibroin fibers).

FIG. 9 is an explanatory diagram schematically showing an example of the manufacturing apparatus for manufacturing artificial hair fibers (artificial fibroin fibers).

FIG. 10 is a scanning electron microscope (SEM) photograph of a surface structure (a skin layer) and an internal structure (a cutting surface) of artificial hair fibers (the artificial fibroin fibers) according to one embodiment.

FIG. 11 is a diagram showing results of measuring a heat contraction rate of the artificial hair fibers (the artificial fibroin fibers) according to one embodiment.

### DESCRIPTION OF EMBODIMENTS

Hereinafter, embodiments for carrying out the present invention will be described in detail. However, the present invention is not limited to the following embodiments.

An artificial hair fiber according to the embodiment is made of an artificial fibroin fiber containing a modified fibroin.

<Modified Fibroin>

The modified fibroin according to the present embodiment is a protein containing a domain sequence represented by Formula 1: [(A)ₙ motif-REP]ₘ or Formula 2: [(A)ₙ motif-REP]ₘ-(A)ₙ motif. In the modified fibroin, an amino acid sequence (an N-terminal sequence and a C-terminal sequence) may be further added to either or both of the N-terminal side and the C-terminal side of the domain sequence. The N-terminal sequence and the C-terminal sequence are typically, but not limited to, regions that do not have a repetition of amino acid motifs characteristic of a fibroin, and that consist of amino acids of about 100 residues.

The “modified fibroin” in the present specification means an artificially produced fibroin (an artificial fibroin). The modified fibroin may be a fibroin in which the domain sequence is different from an amino acid sequence of a naturally occurring fibroin or the same as the amino acid sequence of a naturally occurring fibroin. The “naturally occurring fibroin” referred to in the present specification also is a protein containing a domain sequence represented by Formula 1: [(A)ₙ motif-REP]ₘ or Formula 2: [(A)ₙ motif-REP]ₘ-(A)ₙ motif.

The “modified fibroin” may be a modified fibroin having an amino acid sequence of a naturally occurring fibroin as it is, may be a modified fibroin whose amino acid sequence has been modified based on the amino acid sequence of a naturally occurring fibroin (for example, a modified fibroin whose amino acid sequence has been modified by altering a cloned gene sequence of a naturally occurring fibroin), or may be a modified fibroin that is artificially designed and synthesized independently of a naturally occurring fibroin (for example, a modified fibroin having a desired amino acid sequence by chemically synthesizing a nucleic acid encoding a designed amino acid sequence).

The “domain sequence” in the present specification is an amino acid sequence that produces a crystalline region (typically corresponding to an (A)ₙ motif of an amino acid sequence) and an amorphous region (typically corresponding to REP of an amino acid sequence) unique to a fibroin, and refers to an amino acid sequence represented by Formula 1: [(A)ₙ motif-REP]ₘ or Formula 2: [(A)ₙ motif-REP]ₘ-(A)ₙ motif Here, the (A)ₙ motif represents an amino acid sequence mainly containing alanine residues, and the number of amino acid residues in the (A)ₙ motif is 2 to 27. The number of amino acid residues in the (A)ₙ motif may be an integer of 2 to 20, 4 to 27, 4 to 20, 8 to 20, 10 to 20, 4 to 16, 8 to 16, or 10 to 16. Further, the proportion of the number of alanine residues with respect to the total number of amino acid residues in the (A)ₙ motif may be 40% or more, and may be 60% or more, 70% or more, 80% or more, 83% or more, 85% or more, 86% or more, 90% or more, 95% or more, or 100% (meaning that the (A)ₙ motif is composed of only alanine residues). Among a plurality of (A)ₙ motifs present in the domain sequence, at least seven of the (A)ₙ motifs may be composed only of alanine residues. REP represents an amino acid sequence composed of 2 to 200 amino acid residues. REP may be an amino acid sequence composed of 10 to 200 amino acid residues, and may be an amino acid sequence composed of 10 to 40, 10 to 60, 10 to 80, 10 to 100, 10 to 120, 10 to 140, 10 to 160, or 10 to 180 amino acid residues. m represents an integer of 2 to 300, and may be an integer of 8 to 300, 10 to 300, 20 to 300, 40 to 300, 60 to 300, 80 to 300, 100 to 300, 10 to 200, 20 to 200, 20 to 180, 20 to 160, 20 to 140, or 20 to 120. A plurality of (A)ₙ motifs may have the same amino acid sequence or amino acid sequences different from each other. A plurality of REPs may have the same amino acid sequence or amino acid sequences different from each other.

The modified fibroin according to the present embodiment may be obtained, for example, by subjecting a cloned gene sequence of a naturally occurring fibroin to a modification of an amino acid sequence corresponding to, for example, substitution, deletion, insertion, and/or addition of one or a plurality of amino acid residues. The substitution, deletion, insertion, and/or addition of an amino acid residue may be performed by methods well known to those skilled in the art, such as site-directed mutagenesis. Specifically, the modification may be performed in accordance with the method described in literature such as Nucleic Acid Res. 10, 6487 (1982) and Methods in Enzymology, 100, 448 (1983).

A naturally occurring fibroin is a protein containing a domain sequence represented by Formula 1: [(A)ₙ motif-REP]ₘ or Formula 2: [(A)ₙ motif-REP]ₘ-(A)ₙ motif, and specific examples thereof include a fibroin produced by insects or spiders.

Examples of the fibroin produced by insects include silk proteins produced by silkworms such as Bombyx mori, Bombyx mandarina, Antheraea yamamai, Anteraea pernyi, Eriogyna pyretorum, Pilosamia Cynthia ricini, Samia cynthia, Caligura japonica, Antheraea mylitta, and Antheraea assama; and hornet silk proteins discharged by larvae of Vespa simillima xanthoptera.

More specific examples of the fibroin produced by insects include a silkworm fibroin L chain (GenBank Accession No. M76430 (nucleotide sequence), and AAA27840.1 (amino acid sequence)).

Examples of the fibroin produced by spiders include spider silk proteins produced by spiders belonging to the genus Araneus such as Araneus ventricosus, Araneus diadematus, Araneus pinguis, Araneus pentagrammicus and Araneus nojimai, spiders belonging to the genus Neoscona such as Neoscona scylla, Neoscona nautica, Neoscona adianta and Neoscona scylloides, spiders belonging to the genus Pronus such as Pronous minutes, spiders belonging to the genus Cyrtarachne such as Cyrtarachne bufo and Cyrtarachne inaequalis, spiders belonging to the genus Gasteracantha such as Gasteracantha kuhli and Gasteracantha mammosa, spiders belonging to the genus Ordgarius such as Ordgarius hobsoni and Ordgarius sexspinosus, spiders belonging to the genus Argiope such as Argiope amoena, Argiope minuta and Argiope bruennich, spiders belonging to the genus Arachnura such as Arachnura logio, spiders belonging to the genus Acusilas such as Acusilas coccineus, spiders belonging to the genus Cytophora such as Cytophora moluccensis, Cytophora exanthematica and Cytophora unicolor, spiders belonging to the genus Poltys such as Poltys illepidus, spiders belonging to the genus Cyclosa such as Cyclosa octotuberculata, Cyclosa sedeculata, Cyclosa vallata and Cyclosa atrata, and spiders belonging to the genus Chorizopes such as Chorizopes nipponicus; and spider silk proteins produced by spiders belonging to the genus Tetragnatha such as Tetragnatha praedonia, Tetragnatha maxillosa, Tetragnatha extensa and Tetragnatha squamata, spiders belonging to the genus Leucauge such as Leucauge magnifica, Leucauge blanda and Leucauge subblanda, spiders belonging to the genus Nephila such as Nephila clavata and Nephila pilipes, spiders belonging to the genus Menosira such as Menosira ornata, spiders belonging to the genus Dyschiriognatha such as Dyschiriognatha tenera, spiders belonging to the genus Latrodectus such as Latrodectus mactans, Latrodectus hasseltii, Latrodectus geometricus and Latrodectus tredecimguttatus, and spiders belonging to the family Tetragnathidae such as spiders belonging to the genus Euprosthenops. Examples of the spider silk proteins include traction yarn proteins such as MaSp (MaSp1 and MaSp2) and ADF (ADF3 and ADF4), and MiSp (MiSp1 and MiSp2).

More specific examples of the spider silk protein produced by spiders include fibroin-3 (adf-3) [derived from Araneus diadematus] (GenBank Accession No. AAC47010 (amino acid sequence), U47855 (nucleotide sequence)), fibroin-4 (adf-4) [derived from Araneus diadematus] (GenBank Accession No. AAC47011 (amino acid sequence), U47856 (nucleotide sequence)), dragline silk protein spidroin 1 [derived from Nephila clavipes] (GenBank Accession No. AAC04504 (amino acid sequence), U37520 (nucleotide sequence)), major ampullate spidroin 1 [derived from Latrodectus hesperus] (GenBank Accession No. ABR68856 (amino acid sequence), EF595246 (nucleotide sequence)), dragline silk protein spidroin 2 [derived from Nephila clavata] (GenBank Accession No. AAL32472 (amino acid sequence), AF441245 (nucleotide sequence)), major ampullate spidroin 1 [derived from Euprosthenops australis] (GenBank Accession No. CAJ00428 (amino acid sequence), AJ973155 (nucleotide sequence)) and major ampullate spidroin 2 [Euprosthenops australis] (GenBank Accession No. CAM32249.1 (amino acid sequence), AM490169 (nucleotide sequence)), minor ampullate silk protein 1 [Nephila clavipes] (GenBank Accession No. AAC14589.1 (amino acid sequence), minor ampullate silk protein 2 [Nephila clavipes] (GenBank Accession No. AAC14591.1 (amino acid sequence)), and minor ampullate spidroin-like protein [Nephilengys cruentata] (GenBank Accession No. ABR37278.1 (amino acid sequence)).

More specific examples of the naturally occurring fibroin further include a fibroin whose sequence information is registered in NCBI GenBank. For example, sequences thereof may be confirmed by extracting sequences that has a keyword of spidroin, ampullate, fibroin, “silk and polypeptide”, or “silk and protein” in DEFINITION, among sequences containing INV as DIVISION in sequence information registered in NCBI GenBank; sequences that have a specific character string of a product from CDS; or sequences that have a specific character string described for TISSUE TYPE from SOURCE.

The modified fibroin according to the present embodiment may be a modified silk fibroin (a modified fibroin obtained by modifying an amino acid sequence of a silk protein produced by silkworm), or may be a modified spider silk fibroin (a modified fibroin obtained by modifying an amino acid sequence of a spider silk protein produced by spiders). As the modified fibroin, a modified spider silk fibroin is preferable. The modified spider silk fibroin is also excellent in heat retention, hygroscopic exothermicity, and/or flame retardancy.

Specific examples of the modified fibroin include a modified fibroin derived from a large spinal canal thread protein produced in the major ampullate gland of a spider (first modified fibroin), a modified fibroin having a domain sequence with a reduced content of glycine residues (second modified fibroin), a modified fibroin having a domain sequence with a reduced content of (A)ₙ motifs (third modified fibroin), a modified fibroin with a reduced content of glycine residues and (A)ₙ motifs (fourth modified fibroin), a modified fibroin having a domain sequence containing a region having a locally high hydropathy index (fifth modified fibroin), and a modified fibroin having a domain sequence with a reduced content of glutamine residues (sixth modified fibroin).

Examples of the first modified fibroin include a protein containing a domain sequence represented by Formula 1: [(A)ₙ motif-REP]ₘ. In the first modified fibroin, the number of amino acid residues of the (A)ₙ motif is preferably an integer of 3 to 20, more preferably an integer of 4 to 20, still more preferably an integer of 8 to 20, even still more preferably an integer of 10 to 20, further still preferably an integer of 4 to 16, particularly preferably an integer of 8 to 16, and most preferably an integer of 10 to 16. In the first modified fibroin, the number of amino acid residues constituting REP in Formula 1 is preferably 10 to 200 residues, more preferably 10 to 150 residues, still more preferably 20 to 100 residues, and even still more preferably 20 to 75 residues. In the first modified fibroin, the total number of the glycine residues, the serine residues, and the alanine residues contained in the amino acid sequence represented by Formula 1: [(A)ₙ motif-REP]ₘ is preferably 40% or more, more preferably 60% or more, and still more preferably 70% or more with respect to the total number of amino acid residues.

The first modified fibroin may be a polypeptide that contains a unit of an amino acid sequence represented by Formula 1: [(A)ₙ motif-REP]ₘ, and has the amino acid sequence set forth in any of SEQ ID NOs: 1 to 3 or an amino acid sequence having 90% or more homology with the amino acid sequence set forth in any of SEQ ID NOs: 1 to 3 as the C-terminal sequence.

The amino acid sequence set forth in SEQ ID NO: 1 is the same as the amino acid sequence consisting of 50 amino acid residues at the C-terminal of the amino acid sequence of ADF3 (GI: 1263287, NCBI), the amino acid sequence set forth in SEQ ID NO: 2 is the same as the amino acid sequence obtained by removing 20 residues from the C-terminal of the amino acid sequence set forth in SEQ ID NO: 1, and the amino acid sequence set forth in SEQ ID NO: 3 is the same as the amino acid sequence obtained by removing 29 residues from the C-terminal of the amino acid sequence set forth in SEQ ID NO: 1.

More specific examples of the first modified fibroin include a modified fibroin containing (1-i) the amino acid sequence set forth in SEQ ID NO: 4 (recombinant spider silk protein ADF3KaiLargeNRSH1), or (1-ii) an amino acid sequence having 90% or more sequence identity with the amino acid sequence set forth in SEQ ID NO: 4. The sequence identity is preferably 95% or more.

The amino acid sequence shown in SEQ ID NO 4 is obtained by performing mutation on the amino acid sequence of ADF3 in which an amino acid sequence (SEQ ID NO 5) consisting of a start codon, His10 tag, and HRV3C protease (Human rhinovirus 3C protease) recognition site is added to the N-terminal, such that the 1st to 13th repeat regions are approximately doubled, and the translation terminates at the 1154th amino acid residue. The amino acid sequence of the C-terminal of the amino acid sequence shown in SEQ ID NO 4 is the same as the amino acid sequence shown in SEQ ID NO 3.

The modified fibroin (1-i) may be a modified fibroin consisting of the amino acid sequence set forth in SEQ ID NO: 4.

The second modified fibroin has a domain sequence that has an amino acid sequence with a reduced content of glycine residues, as compared with a naturally occurring fibroin. It can be said that the second modified fibroin is a modified fibroin having an amino acid sequence that corresponds to an amino acid sequence in which, at least one or a plurality of glycine residues in REP are substituted with other amino acid residues, as compared with a naturally occurring fibroin.

The second modified fibroin may have a domain sequence that has an amino acid sequence corresponding to an amino acid sequence in which, in at least one motif sequence selected from GGX and GPGXX (where G represents a glycine residue, P represents a proline residue, and X represents an amino acid residue other than glycine) in REP, at least one glycine residue in one or a plurality of motif sequences is substituted with another amino acid residue, as compared with a naturally occurring fibroin.

In the second modified fibroin, the proportion of the above-described motif sequences in which a glycine residue is substituted with another amino acid residue may be 10% or more with respect to the all motif sequences.

The second modified fibroin may be a modified fibroin that contains a domain sequence represented by Formula 1: [(A)ₙ motif-REP]ₘ, that has an amino acid sequence in which z/w is 30% or more, 40% or more, 50% or more, or 50.9% or more, where z is the total number of amino acid residues of an amino acid sequence consisting of XGX (where X represents an amino acid residue other than glycine) contained in all REPs in the domain sequence excluding the sequence between the (A)ₙ motif positioned closest to the C-terminal side and the C-terminal of the domain sequence; and where w is the total number of amino acid residues in the domain sequence excluding the sequence between the (A)ₙ motif positioned closest to the C-terminal side and the C-terminal of the domain sequence. The number of alanine residues with respect to a total number of amino acid residues in the (A)ₙ motif may be 83% or more, but is preferably 86% or more, more preferably 90% or more, still more preferably 95% or more, and even still more preferably 100% (which means that the (A)ₙ motif consists only of alanine residues).

The second modified fibroin is preferably a modified fibroin in which one glycine residue of the GGX motif is substituted with another amino acid residue to increase the content ratio of the amino acid sequence consisting of XGX. In the second modified fibroin, the content ratio of the amino acid sequence consisting of GGX in the domain sequence is preferably 30% or less, more preferably 20% or less, still more preferably 10% or less, even still more preferably 6% or less, further still more preferably 4% or less, and particularly preferably 2% or less. The content ratio of the amino acid sequence consisting of GGX in the domain sequence can be calculated by the same method as the method for calculating a content ratio (z/w) of an amino acid sequence consisting of XGX below.

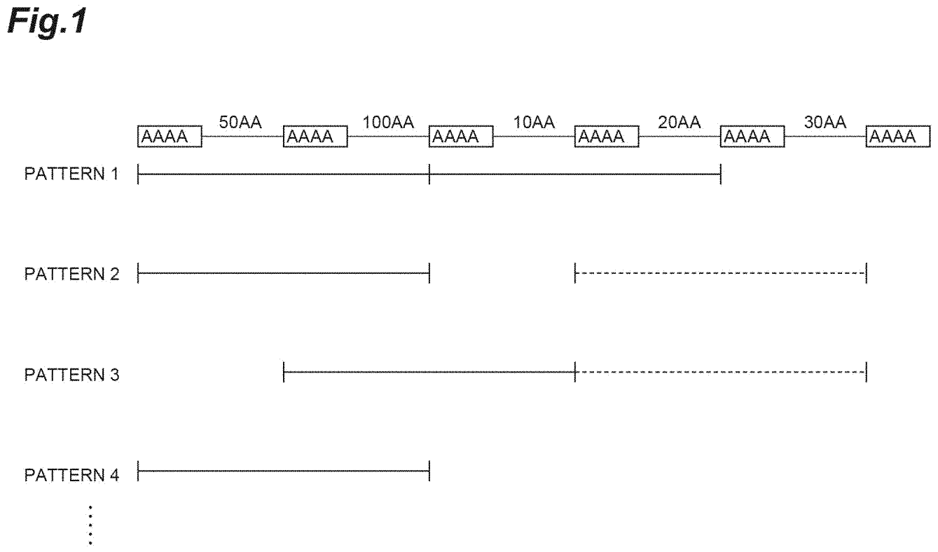

The method for calculating z/w will be described in more detail. First, in a fibroin (a modified fibroin or a naturally occurring fibroin) containing a domain sequence represented by Formula 1: [(A)ₙ motif-REP]ₘ, an amino acid sequence consisting of XGX is extracted from all REPs contained in the domain sequence excluding the sequence between the (A)ₙ motif positioned closest to the C-terminal side and the C-terminal of the domain sequence. The total number of amino acid residues constituting XGX is z. For example, in a case where 50 amino acid sequences consisting of XGX (without an overlap) are extracted, z is 50×3=150. Further, for example, in a case where there is X contained in two XGXs (X at the center), as in the case of an amino acid sequence consisting of XGXGX, the calculation is performed by deducting the overlap (in the case of XGXGX, 5 amino acid residues). w is the total number of amino acid residues contained in the domain sequence excluding the sequence between the (A)ₙ motif positioned closest to the C-terminal side and the C-terminal of the domain sequence. For example, in the case of the domain sequence illustrated in FIG. 1, w is 4+50+4+100+4+10+4+20+4+30=230 (the (A)ₙ motif positioned closest to the C-terminal side is excluded.). Next, z/w (%) can be calculated by dividing z by w.

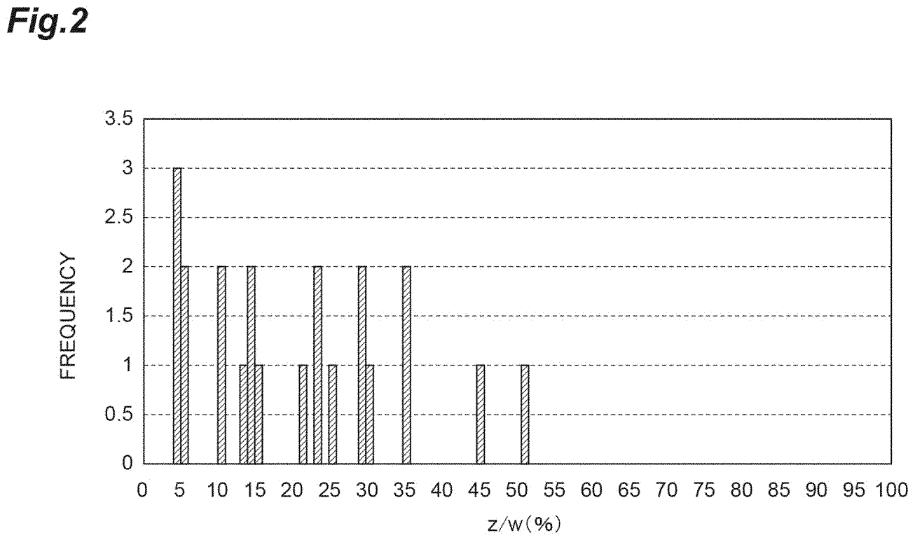

Here, z/w in a naturally occurring fibroin will be described. First, fibroins with amino acid sequence information registered in NCBI GenBank was checked by the exemplified method as described above, and 663 types of fibroins (of which 415 types were fibroins derived from spiders) were extracted. Among all the extracted fibroins, z/w was calculated from an amino acid sequence of a naturally occurring fibroin that contains a domain sequence represented by Formula 1: [(A)ₙ motif-REP]ₘ, and that has a content ratio of the amino acid sequence consisting of GGX in the fibroin of 6% or less, using the above-mentioned calculation method. The results are shown in FIG. 2. In FIG. 2, the horizontal axis represents z/w (%), and the vertical axis represents frequency. As apparent from FIG. 2, z/w in all the naturally occurring fibroin is less than 50.9% (highest value: 50.86%).

In the second modified fibroin, z/w is preferably 50.9% or more, more preferably 56.1% or more, still more preferably 58.7% or more, even still more preferably 70% or more, and further still preferably 80% or more. The upper limit of z/w is not particularly limited, but may be, for example, 95% or less.

The second modified fibroin may be obtained, for example, by modifying a cloned gene sequence of a naturally occurring fibroin such that at least a part of the nucleotide sequence encoding a glycine residue is substituted to encode another amino acid residue. In this case, one glycine residue in the GGX motif and the GPGXX motif may be selected as the glycine residue to be modified, and also, substitution may be performed so that z/w becomes 50.9% or more. In addition, the second modified fibroin may also be obtained, for example, by designing an amino acid sequence that satisfies the above embodiment based on the amino acid sequence of a naturally occurring fibroin, and chemically synthesizing a nucleic acid encoding the designed amino acid sequence. In any case, in addition to the modification that corresponds to substitution of the glycine residue in REP in the amino acid sequence of a naturally occurring fibroin with another amino acid residue, a modification of the amino acid sequence that corresponds to substitution, deletion, insertion, and/or addition of one or a plurality of amino acid residues may further be performed.

The above another amino acid residue is not particularly limited as long as it is an amino acid residue other than the glycine residue, but is preferably a hydrophobic amino acid residue such as a valine (V) residue, a leucine (L) residue, a isoleucine (I) residue, a methionine (M) residue, a proline (P) residue, a phenylalanine (F) residue, and a tryptophan (W) residue, or a hydrophilic amino acid residues such a glutamine (Q) residue, an asparagine (N) residue, a serine (S) residue, a lysine (K) residue, and a glutamic acid (E) residue, more preferably the valine (V) residue, the leucine (L) residue, the isoleucine (I) residue, the phenylalanine (F) residue, and the glutamine (Q) residue, and still more preferably the glutamine (Q) residue.

More specific examples of the second modified fibroin include a modified fibroin containing (2-i) the amino acid sequence set forth in SEQ ID NO: 6 (Met-PRT380), SEQ ID NO: 7 (Met-PRT410), SEQ ID NO: 8 (Met-PRT525), or SEQ ID NO: 9 (Met-PRT799), or (2-ii) an amino acid sequence having 90% or more sequence identity with the amino acid sequence set forth in SEQ ID NO: 6, SEQ ID NO: 7, SEQ ID NO: 8, or SEQ ID NO: 9.

The modified fibroin (2-i) will be described. The amino acid sequence set forth in SEQ ID NO: 6 is an amino acid sequence in which all GGXs in REP of the amino acid sequence set forth in SEQ ID NO: 10 (Met-PRT313) corresponding to a naturally occurring fibroin are substituted with GQXs. The amino acid sequence set forth in SEQ ID NO: 7 is an amino acid sequence in which every third (A)ₙ motif from the N-terminal side to the C-terminal side is deleted from the amino acid sequence set forth in SEQ ID NO: 6, and further, one [(A)ₙ motif-REP] is inserted before the C-terminal sequence. The amino acid sequence set forth in SEQ ID NO: 8 is an amino acid sequence in which two alanine residues are inserted at the C-terminal side of each (A)ₙ motif of the amino acid sequence set forth in SEQ ID NO: 7, and further some of the glutamine (Q) residues are substituted with a serine (S) residue, and some of the amino acids on the C-terminal side are deleted so that the molecular weight is almost the same as that of SEQ ID NO 7. The amino acid sequence set forth in SEQ ID NO: 9 is an amino acid sequence in which a predetermined hinge sequence and a His tag sequence are added to the C-terminal of a sequence having four-time repetition of a region of 20 domain sequences present in the amino acid sequence set forth in SEQ ID NO: 7 (however, with several amino acid residues on the C-terminal side of the region are substituted).

The value of z/w in the amino acid sequence set forth SEQ ID NO: 10 (corresponding to a naturally occurring fibroin) is 46.8%. The values of z/w in the amino acid sequence set forth in SEQ ID NO: 6, the amino acid sequence set forth in SEQ ID NO: 7, the amino acid sequence set forth in SEQ ID NO: 8, and the amino acid sequence set forth in SEQ ID NO: 9 are 58.7%, 70.1%, 66.1%, and 70.0%, respectively. In addition, the values of x/y at the Giza ratio (which will be described later) of 1:1.8 to 11.3 in the amino acid sequences set forth in SEQ ID NO: 10, SEQ ID NO: 6, SEQ ID NO: 7, SEQ ID NO: 8, and SEQ ID NO: 9 are 15.0%, 15.0%, 93.4%, 92.7%, and 89.8%, respectively.

The modified fibroin (2-i) may be a modified fibroin consisting of the amino acid sequence set forth in SEQ ID NO: 6, SEQ ID NO: 7, SEQ ID NO: 8, or SEQ ID NO: 9.

The modified fibroin (2-ii) is a modified fibroin containing an amino acid sequence having 90% or more sequence identity with the amino acid sequence set forth in SEQ ID NO: 6, SEQ ID NO: 7, SEQ ID NO: 8, or SEQ ID NO: 9. The modified fibroin (2-ii) also is a protein containing a domain sequence represented by Formula 1: [(A)ₙ motif-REP]ₘ. The sequence identity is preferably 95% or more.

The modified fibroin (2-ii) preferably has 90% or more sequence identity with the amino acid sequence set forth in SEQ ID NO: 6, SEQ ID NO: 7, SEQ ID NO: 8, or SEQ ID NO: 9, and that z/w is 50.9% or more, where z is the total number of amino acid residues in the amino acid sequence consisting of XGX (where X represents an amino acid residue other than glycine) contained in REP; and where w is the total number of amino acid residues in REP in the domain sequence.

The second modified fibroin may contain a tag sequence at either or both of the N-terminal and C-terminal. This makes it possible to isolate, immobilize, detect, and visualize the modified fibroin.

The tag sequence may be, for example, an affinity tag utilizing specific affinity (binding property, affinity) with other molecules. Specific examples of the affinity tag include a histidine tag (His tag).

The His tag is a short peptide in which about 4 to 10 histidine residues are arranged and has a property of specifically binding to a metal ion such as nickel, and thus, it can be used for isolation of a modified fibroin by a chelating metal chromatography. Specific examples of the tag sequence include an amino acid sequence set forth in SEQ ID NO: 11 (amino acid sequence containing a His tag sequence and a hinge sequence).

In addition, a tag sequence such as a glutathione-S-transferase (GST) that specifically binds to glutathione and a maltose binding protein (MBP) that specifically binds to maltose may also be used.

Furthermore, an “epitope tag” utilizing an antigen-antibody reaction may also be used. By adding a peptide (an epitope) showing antigenicity as a tag sequence, an antibody against the epitope can be bound. Examples of the epitope tag include an HA (peptide sequence of hemagglutinin of an influenza virus) tag, a myc tag, and a FLAG tag. The modified fibroin can be easily purified with high specificity by utilizing the epitope tag.

It is also possible to further use a tag sequence which can be cleaved with a specific protease. By treating a protein adsorbed via the tag sequence with a protease, it is also possible to recover a modified fibroin cleaved from the tag sequence.

More specific examples of the modified fibroin containing the tag sequence include a modified fibroin containing (2-iii) the amino acid sequence set forth in SEQ ID NO: 12 (PRT380), SEQ ID NO: 13 (PRT410), SEQ ID NO: 14 (PRT525), or SEQ ID NO: 15 (PRT799), or (2-iv) an amino acid sequence having 90% or more sequence identity with the amino acid sequence set forth in SEQ ID NO: 12, SEQ ID NO: 13, SEQ ID NO: 14, or SEQ ID NO: 15.

The amino acid sequences set forth in SEQ ID NO: 16 (PRT313), SEQ ID NO: 12, SEQ ID NO: 13, SEQ ID NO: 14, and SEQ ID NO: 15 are amino acid sequences in which the amino acid sequence (containing a His tag sequence and a hinge sequence) set forth in SEQ ID NO: 11 is added to the N-terminal of the amino acid sequences set forth in SEQ ID NO: 10, SEQ ID NO: 6, SEQ ID NO: 7, SEQ ID NO: 8, and SEQ ID NO: 9, respectively.

The modified fibroin (2-iii) may be a modified fibroin consisting of the amino acid sequence set forth in SEQ ID NO: 12, SEQ ID NO: 13, SEQ ID NO: 14, or SEQ ID NO: 15.

The modified fibroin (2-iv) is a modified fibroin containing an amino acid sequence having 90% or more sequence identity with the amino acid sequence set forth in SEQ ID NO: 12, SEQ ID NO: 13, SEQ ID NO: 14, or SEQ ID NO: 15. The modified fibroin (2-iv) also is a protein containing a domain sequence represented by Formula 1: [(A)ₙ motif-REP]ₘ. The sequence identity is preferably 95% or more.

It is preferable that the modified fibroin (2-iv) has 90% or more sequence identity with the amino acid sequence set forth in SEQ ID NO: 12, SEQ ID NO: 13, SEQ ID NO: 14, or SEQ ID NO: 15, and that z/w is 50.9% or more, where z is the total number of amino acid residues of the amino acid sequence consisting of XGX (where X represents an amino acid residue other than glycine) contained in REP; and where w is the total number of amino acid residues in REP in the domain sequence.

The second modified fibroin may contain a secretory signal for releasing the protein produced in a recombinant protein production system to the outside of the host. The sequence of the secretory signal may be set as appropriate, depending on the type of the host.

The third modified fibroin has a domain sequence that has an amino acid sequence with a reduced content of (A)ₙ motifs, as compared with a naturally occurring fibroin. It can be said that the domain sequence of the third modified fibroin is a domain sequence having an amino acid sequence that corresponds to an amino acid sequence in which at least one or a plurality of the (A)ₙ motifs are deleted, as compared with a naturally occurring fibroin.

The third modified fibroin may be a modified fibroin having an amino acid sequence that corresponds to an amino acid sequence in which 10% to 40% of the (A)ₙ motifs are deleted from a naturally occurring fibroin.

The domain sequence of the third modified fibroin may be a domain sequence having an amino acid sequence that corresponds to an amino acid sequence in which at least one (A)ₙ motif for every one to three (A)ₙ motifs from the N-terminal side to the C-terminal side is deleted, as compared with a naturally occurring fibroin.

The domain sequence of the third modified fibroin may be a domain sequence having an amino acid sequence that corresponds to an amino acid sequence in which at least deletion of two consecutive (A)ₙ motifs and deletion of one (A)ₙ motif are repeated in this order from the N-terminal side to the C-terminal side, as compared with a naturally occurring fibroin.

The domain sequence of the third modified fibroin may be a domain sequence having an amino acid sequence that corresponds to an amino acid sequence in which, at least, every third (A)ₙ motif from the N-terminal side to the C-terminal side is deleted.

The third modified fibroin may be a modified fibroin that contains a domain sequence represented by Formula 1: [(A)ₙ motif-REP]ₘ, and that has an amino acid sequence in which, when the numbers of amino acid residues of REP of two adjacent [(A)ₙ motif-REP] units are sequentially compared from the N-terminal side to the C-terminal side, x/y is 20% or more, 30% or more, 40% or more, or 50% or more, where x is a maximum value of a total value obtained by adding the numbers of amino acid residues of two adjacent [(A)ₙ motif-REP] units wherein when the number of amino acid residues of one REP having less amino acid residues is taken as 1, the ratio of the number of amino acids residues of the other REP satisfies 1.8 to 11.3; and where y is a total number of amino acid residues of the domain sequence. The number of alanine residues with respect to a total number of amino acid residues in the (A)ₙ motif may be 83% or more, but is preferably 86% or more, more preferably 90% or more, still more preferably 95% or more, and even still more preferably 100% (which means that the (A)ₙ motif consists only of alanine residues).

A method for calculating x/y will be described in more detail with reference to FIG. 1. FIG. 1 illustrates a domain sequence obtained by removing an N-terminal sequence and a C-terminal sequence from a modified fibroin. The domain sequence has a sequence of, from the N-terminal side (left side), (A)ₙ motif-the first REP (50 amino acid residues)-(A)ₙ motif-the second REP (100 amino acid residues)-(A)ₙ motif-the third REP (10 amino acid residues)-(A)ₙ motif-the fourth REP (20 amino acid residues)-(A)ₙ motif-the fifth REP (30 amino acid residues)-(A)ₙ motif.

The two adjacent [(A)ₙ motif-REP] units are sequentially selected from the N-terminal side to the C-terminal side so that there is no overlap. There may be [(A)ₙ motif-REP] unit that is not selected. FIG. 1 illustrates Pattern 1 (a comparison of the first REP and the second REP, and a comparison of the third REP and the fourth REP), Pattern 2 (a comparison of the first REP and the second REP, and a comparison of the fourth REP and the fifth REP), Pattern 3 (a comparison of the second REP and the third REP, and a comparison of the fourth REP and the fifth REP), and Pattern 4 (a comparison of the first REP and the second REP). There are selection methods other than these.

Next, for each pattern, the number of amino acid residues of each REP in the selected adjacent two [(A)ₙ motif-REP] units is compared. The comparison is performed by determining the ratio, relative to one REP having less amino acid residues taken as 1, of the number of amino acid residues of the other REP. For example, in a case of comparing the first REP (50 amino acid residues) and the second REP (100 amino acid residues), the ratio of the number of amino acid residues of the second REP is 100/50=2, where the first REP having less amino acid residues is taken as 1. Similarly, in a case of comparing the fourth REP (20 amino acid residues) and the fifth REP (30 amino acid residues), the ratio of the number of amino acid residues of the fifth REP is 30/20=1.5, where the fourth REP having less amino acid residues is taken as 1.

In FIG. 1, a set of [(A)ₙ motif-REP] units in which, where one REP having less amino acid residues is taken as 1, the ratio of the number of amino acid residues of the other REP is 1.8 to 11.3, is indicated by a solid line. In the present specification, this ratio is referred to as a Giza ratio. A set of [(A)ₙ motif-REP] units in which, where a REP having less amino acid residues is taken as 1, the ratio of the number of amino acid residues of the other REP is less than 1.8 or more than 11.3, is indicated by a dotted line.

In each pattern, the numbers of all amino acid residues (not only REP but also the number of amino acid residues in the (A)ₙ motif) of the two adjacent [(A)ₙ motif-REP] units indicated by a solid line are added. Then, the total values obtained by addition are compared, and the total value of the pattern having the highest total value (a maximum value of the total value) is defined as x. In the example illustrated in FIG. 1, the total value of Pattern 1 is the maximum.

Next, x/y (%) can be calculated by dividing x by the total number y of the amino acid residues of the domain sequence.

In the third modified fibroin, x/y is preferably 50% or more, more preferably 60% or more, still more preferably 65% or more, even still more preferably 70% or more, further still preferably 75% or more, and particularly preferably 80% or more. The upper limit of x/y is not particularly limited, but may be, for example, 100% or less. In a case where the Giza ratio is 1:1.9 to 11.3, x/y is preferably 89.6% or more, in a case where the Giza ratio is 1:1.8 to 3.4, x/y is more preferably 77.1% or more, in a case where the Giza ratio is 1:1.9 to 8.4, x/y is still more preferably 75.9% or more, and in a case where the Giza ratio is 1:1.9 to 4.1, x/y is even still more preferably 64.2% or more.

In a case where the third modified fibroin is a modified fibroin in which at least seven of a plurality of (A)ₙ motifs present in the domain sequence are composed only of alanine residues, x/y is preferably 46.4% or more, more preferably 50% or more, still more preferably 55% or more, even still more preferably 60% or more, further still preferably 70% or more, and particularly preferably 80% or more. The upper limit of x/y is not particularly limited and it only needs to be 100% or less.

Here, x/y in a naturally occurring fibroin will be described.

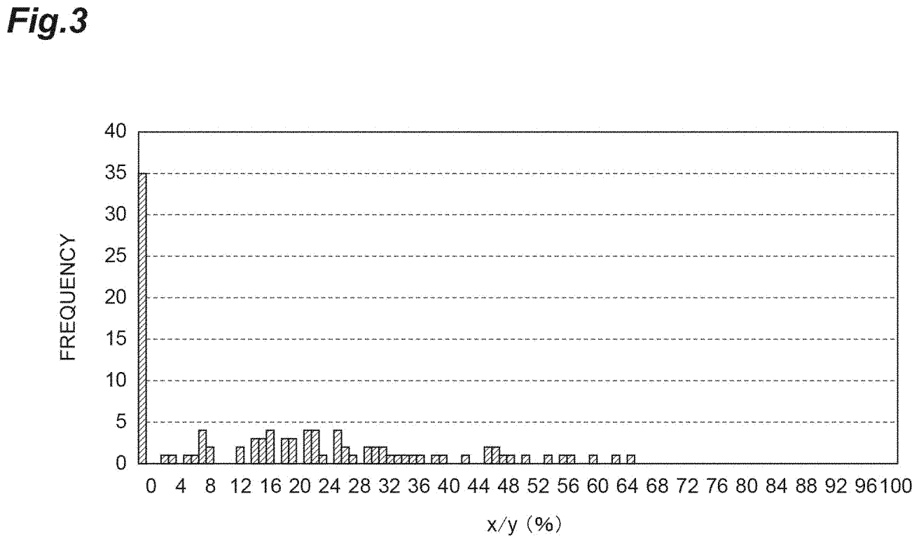

First, fibroins with amino acid sequence information registered in NCBI GenBank was checked by the exemplified method as described above, and 663 types of fibroins (of which 415 types were fibroins derived from spiders) were extracted. Among all the extracted fibroins, x/y was calculated from an amino acid sequence of a naturally occurring fibroin constituted with a domain sequence represented by Formula 1: [(A)ₙ motif-REP]ₘ, using the above-mentioned calculation method. The results in a case where the Giza ratio is 1:1.9 to 4.1 are shown in FIG. 3.

In FIG. 3, the horizontal axis represents x/y (%) and the vertical axis represents a frequency. As apparent from FIG. 3, x/y in all the naturally occurring fibroin is less than 64.2% (highest value: 64.14%).

The third modified fibroin may be obtained, for example, by deleting one or a plurality sequences encoding (A)ₙ motif from a cloned gene sequence of a naturally occurring fibroin so that x/y is 64.2% or more. In addition, the third modified fibroin may also be obtained, for example, by designing an amino acid sequence that corresponds to an amino acid sequence in which one or a plurality (A)ₙ motifs are deleted from an amino acid sequence of a naturally occurring fibroin so that x/y becomes 64.2% or more, and chemically synthesizing a nucleic acid encoding the designed amino acid sequence. In any case, in addition to the modification that corresponds to deletion of the (A)ₙ motif from an amino acid sequence of a naturally occurring fibroin, a modification of the amino acid sequence that corresponds to substitution, deletion, insertion, and/or addition of one or a plurality of amino acid residues may further be performed.

More specific examples of the third modified fibroin include a modified fibroin containing an amino acid sequence having (3-i) the amino acid sequence set forth in SEQ ID NO: 17 (Met-PRT399), SEQ ID NO: 7 (Met-PRT410), SEQ ID NO: 8 (Met-PRT525), or SEQ ID NO: 9 (Met-PRT799), or (3-ii) an amino acid sequence having 90% or more sequence identity with the amino acid sequence set forth in SEQ ID NO: 17, SEQ ID NO: 7, SEQ ID NO: 8, or SEQ ID NO: 9.

The modified fibroin (3-i) will be described. The amino acid sequence set forth in SEQ ID NO: 17 is an amino acid sequence in which every third (A)ₙ motif from the N-terminal side to the C-terminal side is deleted from the amino acid sequence set forth in SEQ ID NO: 10 (Met-PRT313) corresponding to a naturally occurring fibroin, and further, one [(A)ₙ motif-REP] is inserted before the C-terminal sequence. The amino acid sequences set forth in SEQ ID NO: 7, SEQ ID NO: 8, or SEQ ID NO: 9 are as described for the second modified fibroin.

The value of x/y at a Giza ratio of 1:1.8 to 11.3 in the amino acid sequence set forth in SEQ ID NO: 10 (corresponding to a naturally occurring fibroin) is 15.0%. Both the values of x/y in the amino acid sequence set forth in SEQ ID NO: 17 and the amino acid sequence set forth in SEQ ID NO: 7 are 93.4%. The value of x/y in the amino acid sequence set forth in SEQ ID NO: 8 is 92.7%. The value of x/y in the amino acid sequence set forth in SEQ ID NO: 9 is 89.8%. The values of z/w in the amino acid sequences set forth in SEQ ID NO: 10, SEQ ID NO: 17, SEQ ID NO: 7, SEQ ID NO: 8, and SEQ ID NO: 9 are 46.8%, 56.2%, 70.1%, 66.1%, and 70.0%, respectively.

The modified fibroin (3-i) may be a modified fibroin consisting of the amino acid sequence set forth in SEQ ID NO: 17, SEQ ID NO: 7, SEQ ID NO: 8, or SEQ ID NO: 9.

The modified fibroin (3-ii) is a modified fibroin containing an amino acid sequence having 90% or more sequence identity with the amino acid sequence set forth in SEQ ID NO: 17, SEQ ID NO: 7, SEQ ID NO: 8, or SEQ ID NO: 9. The modified fibroin (3-ii) also is a protein containing a domain sequence represented by Formula 1: [(A)ₙ motif-REP]ₘ. The sequence identity is preferably 95% or more.

It is preferable that the modified fibroin (3-ii) has 90% or more sequence identity with the amino acid sequence set forth in SEQ ID NO: 17, SEQ ID NO: 7, SEQ ID NO: 8, or SEQ ID NO: 9, and that, when the numbers of amino acid residues of REP of two adjacent [(A)ₙ motif-REP] units are sequentially compared from the N-terminal side to the C-terminal side, x/y is 64.2% or more, where x is a maximum value of a total value obtained by adding the numbers of amino acid residues of two adjacent [(A)ₙ motif-REP] units wherein when the number of amino acid residues of one REP having less amino acid residues is taken as 1, the ratio of the number of amino acids residues of the other REP satisfies 1.8 to 11.3 (the Giza ratio is 1:1.8 to 11.3); and where y is a total number of amino acid residues of the domain sequence.

The third modified fibroin may contain a tag sequence described above at either or both of the N-terminal and C-terminal.

A more specific example of the modified fibroin containing a tag sequence may be a modified fibroin containing (3-iii) the amino acid sequence set forth in SEQ ID NO: 18 (PRT399), SEQ ID NO: 13 (PRT410), SEQ ID NO: 14 (PRT525), or SEQ ID NO: 15 (PRT799), or (3-iv) an amino acid sequence having 90% or more sequence identity with the amino acid sequence set forth in SEQ ID NO: 18, SEQ ID NO: 13, SEQ ID NO: 14, or SEQ ID NO: 15.

The amino acid sequences set forth in SEQ ID NO: 18, SEQ ID NO: 13, SEQ ID NO: 14, and SEQ ID NO: 15 are amino acid sequences in which the amino acid sequence (containing a His tag sequence and a hinge sequence) set forth in SEQ ID NO: 11 is added to the N-terminal of the amino acid sequences set forth in SEQ ID NO: 17, SEQ ID NO: 7, SEQ ID NO: 8, and SEQ ID NO: 9, respectively.

The modified fibroin (3-iii) may be a modified fibroin consisting of the amino acid sequence set forth in SEQ ID NO: 18, SEQ ID NO: 13, SEQ ID NO: 14, or SEQ ID NO: 15.

The modified fibroin (3-iv) is a modified fibroin containing an amino acid sequence having 90% or more sequence identity with the amino acid sequence set forth in SEQ ID NO: 18, SEQ ID NO: 13, SEQ ID NO: 14, or SEQ ID NO: 15. The modified fibroin (3-iv) also is a protein containing a domain sequence represented by Formula 1: [(A)ₙ motif-REP]ₘ. The sequence identity is preferably 95% or more.

It is preferable that the modified fibroin (3-iv) has 90% or more sequence identity with the amino acid sequence set forth in SEQ ID NO: 18, SEQ ID NO: 13, SEQ ID NO: 14 or SEQ ID NO: 15, and that, when the numbers of amino acid residues of REP of two adjacent [(A)ₙ motif-REP] units are sequentially compared from the N-terminal side to the C-terminal side, x/y is 64.2% or more, where x is a maximum value of a total value obtained by adding the numbers of amino acid residues of two adjacent [(A)ₙ motif-REP] units wherein when the number of amino acid residues of one REP having less amino acid residues is taken as 1, the ratio of the number of amino acids residues of the other REP satisfies 1.8 to 11.3; and where y is a total number of amino acid residues of the domain sequence.

The third modified fibroin may contain a secretory signal for releasing the protein produced in a recombinant protein production system to the outside of the host. The sequence of the secretory signal may be set as appropriate, depending on the type of the host.

The fourth modified fibroin has a domain sequence that has an amino acid sequence with a reduced content of glycine residues, in addition to the reduced content of (A)ₙ motifs, as compared with a naturally occurring fibroin. It can be said that the domain sequence of the fourth modified fibroin is a domain sequence further having an amino acid sequence that corresponds to an amino acid sequence in which at least one or a plurality of glycine residues in REP are substituted with other amino acid residues, in addition to the amino acid sequence in which at least one or a plurality of the (A)ₙ motifs are deleted, as compared with a naturally occurring fibroin. That is, the fourth modified fibroin is a modified fibroin having both the characteristics of the second modified fibroin and the characteristics of the third modified fibroin described above. Specific embodiments and the like are as described for the second modified fibroin and the third modified fibroin.

More specific examples of the fourth modified fibroin include a modified fibroin containing (4-i) the amino acid sequence set forth in SEQ ID NO: 7 (Met-PRT410), SEQ ID NO: 8 (Met-PRT525), SEQ ID NO: 9 (Met-PRT799), SEQ ID NO: 13 (PRT410), SEQ ID NO: 14 (PRT525), or SEQ ID NO: 15 (PRT799), or (4-ii) an amino acid sequence having 90% or more sequence identity with the amino acid sequence set forth in SEQ ID NO: 7, SEQ ID NO: 8, SEQ ID NO: 9, SEQ ID NO: 13, SEQ ID NO: 14, or SEQ ID NO: 15. Specific embodiments of the modified fibroin containing the amino acid sequence set forth in SEQ ID NO: 7, SEQ ID NO: 8, SEQ ID NO: 9, SEQ ID NO: 13, SEQ ID NO: 14, or SEQ ID NO: 15 are as described above.

The fifth modified fibroin may have a domain sequence that has an amino acid sequence containing a region having a locally high hydropathy index, which corresponds to an amino acid sequence in which one or a plurality of amino acid residues in REP are substituted with an amino acid residue having a high hydropathy index and/or one or a plurality of amino acid residues having a high hydropathy index is inserted into REP, as compared with a naturally occurring fibroin.

It is preferable that the region having a locally high hydropathy index is composed of 2 to 4 consecutive amino acid residues.

It is more preferable that the amino acid residues having a high hydropathy index are selected from isoleucine (I), valine (V), leucine (L), phenylalanine (F), cysteine (C), methionine (M), and alanine (A).

The fifth modified fibroin may further have a modification of an amino acid sequence that corresponds to substitution, deletion, insertion, and/or addition of one or a plurality of amino acid residues, as compared with a naturally occurring fibroin, in addition to the modification that corresponds to substitution of one or a plurality of amino acid residues in REP with amino acid residues having a high hydropathy index and/or insertion of one or a plurality of amino acid residues having a high hydropathy index into REP, as compared with a naturally occurring fibroin.

The fifth modified fibroin may be obtained, for example, from a cloned gene sequence of a naturally occurring fibroin, by substituting one or a plurality of hydrophilic amino acid residues in REP (for example, amino acid residues having a negative hydropathy index) with a hydrophobic amino acid residue (for example, amino acid residues having a positive hydropathy index), and/or inserting one or a plurality of hydrophobic amino acid residues into REP. In addition, the fifth modified fibroin may also be obtained, for example, by designing an amino acid sequence that corresponds to an amino acid sequence in which one or a plurality of hydrophilic amino acid residues in REP are substituted with hydrophobic amino acid residues and/or one or a plurality of hydrophobic amino acid residues are inserted into REP, from an amino acid sequence of a naturally occurring fibroin, and chemically synthesizing a nucleic acid encoding the designed amino acid sequence. In any case, in addition to the modification that corresponds to substitution of one or a plurality of hydrophilic amino acid residues in REP with hydrophobic amino acid residues and/or insertion of one or a plurality of hydrophobic amino acid residues into REP in an amino acid sequence of a naturally occurring fibroin, a modification of an amino acid sequence that corresponds to substitution, deletion, insertion, and/or addition of one or a plurality of amino acid residues may further be performed.

The fifth modified fibroin may be a modified fibroin that contains a domain sequence represented by Formula 1: [(A)ₙ motif-REP]ₘ, and that has an amino acid sequence in which p/q is 6.2% or more, where p is the total number of amino acid residues contained in a region where an average value of hydropathy indices of the four consecutive amino acid residues is 2.6 or more in all REPs contained in the domain sequence excluding the sequence between the (A)ₙ motif positioned closest to the C-terminal side and the C-terminal of the domain sequence; and where q is the total number of amino acid residues contained in the domain sequence excluding the sequence between the (A)ₙ motif positioned closest to the C-terminal side and the C-terminal of the domain sequence.

With regard to the hydropathy index of an amino acid residue, known index from (Hydropathy index: Kyte J, & Doolittle R (1982) “A simple method for displaying the hydropathic character of a protein”, J. Mol. Biol., 157, pp. 105-132) may be used. Specifically, the hydropathy index (hereinafter also referred to as “HI”) of each amino acid is as shown in Table 1 below.

TABLE 1

| Amino acid | HI |
|---|---|
| Isoleucine (Ile) | 4.5 |
| Valine (Val) | 4.2 |
| Leucine (Leu) | 3.8 |
| Phenylalanine (Phe) | 2.8 |
| Cysteine (Cys) | 2.5 |
| Methionine (Met) | 1.9 |
| Alanine (Ala) | 1.8 |
| Glycine (Gly) | −0.4 |
| Threonine (Thr) | −0.7 |
| Serine (Ser) | −0.8 |
| Tryptophan (Trp) | −0.9 |
| Tyrosine (Tyr) | −1.3 |
| Proline (Pro) | −1.6 |
| Histidine (His) | −3.2 |
| Asparagine (Asn) | −3.5 |
| Aspartic acid (Asp) | −3.5 |
| Glutamine (Gin) | −3.5 |
| Glutamic acid (Glu) | −3.5 |
| Lysine (Lys) | −3.9 |
| Arginine (Arg) | −4.5 |

A method for calculating p/q will be described in more detail. For calculation, a sequence (hereinafter also referred to as a “Sequence A”) obtained by removing, from domain sequence represented by Formula 1: [(A)ₙ motif-REP]ₘ, the sequence between the (A)ₙ motif positioned closest to the C-terminal side and the C-terminal of the domain sequence is used. First, in all REPs contained in Sequence A, average values of hydropathy indices of the four consecutive amino acid residues are calculated. The average value of the hydropathy indices is obtained by dividing the sum of HI of each of the amino acid residues contained in the four consecutive amino acid residues by 4 (the number of amino acid residues). The average value of the hydropathy indices is obtained for all four consecutive amino acid residues (each of the amino acid residues is used for calculating the average value 1 to 4 times). Next, a region where the average value of the hydropathy indices of the four consecutive amino acid residues is 2.6 or more is identified. Even if a certain amino acid residues correspond to a plurality of the “four consecutive amino acid residues having an average value of the hydropathy indices of 2.6 or more”, the amino acid residue is counted as one amino acid residue in the region. The total number of amino acid residues contained in the region is p. Further, the total number of amino acid residues contained in Sequence A is q.

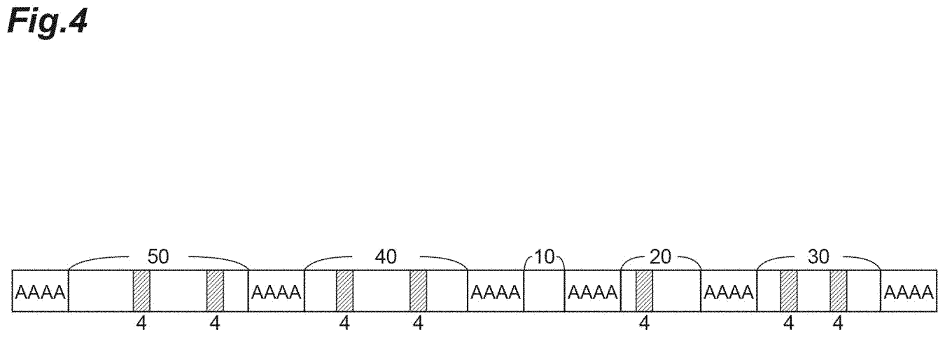

For example, in a case where 20 “four consecutive amino acid residues having an average value of the hydropathy indices of 2.6 or more” are extracted (without an overlap), there are 20 of the four consecutive amino acid residues (without an overlap) in the region in which the average value of the hydropathy indices of the four consecutive amino acid residues is 2.6 or more, and thus p is 20×4=80. In addition, for example, in a case where two “four consecutive amino acid residues having an average value of the hydropathy indices of 2.6 or more” overlap by only one amino acid residue, seven amino acid residues are contained in the region in which the average value of the hydropathy indices of the four consecutive amino acid residues is 2.6 or more (p=2×4−1=7; “−1” is deduction for overlap). For example, in a case of the domain sequence shown in FIG. 4, since there are seven “four consecutive amino acid residues having an average value of the hydropathy indices of 2.6 or more” without an overlap, p is 7×4=28. Further, for example, in a case of the domain sequence illustrated in FIG. 4, q is 4+50+4+40+4+10+4+20+4+30=170 (the (A)ₙ motif present at the end of the C-terminal side is not included). Next, p/q (%) can be calculated by dividing p by q. In the case of FIG. 4, p/q (%) is 28/170=16.47%.

In the fifth modified fibroin, p/q is preferably 6.2% or more, more preferably 7% or more, still more preferably 10% or more, even still more preferably 20% or more, and further still preferably 30% or more. The upper limit of p/q is not particularly limited, but may be, for example, 45% or less.

The fifth modified fibroin may be obtained, for example, by modifying an amino acid sequence of a cloned a naturally occurring fibroin to an amino acid sequence containing a region having a locally high hydropathy index, by substituting one or a plurality of hydrophilic amino acid residues in REP (for example, amino acid residues having a negative hydropathy index) with hydrophobic amino acid residues (for example, amino acid residues having a positive hydropathy index), and/or inserting one or a plurality of hydrophobic amino acid residues into REP, so as to satisfy the above condition of p/q. In addition, the modified fibroin may also be obtained, for example, by designing an amino acid sequence satisfying the above-described condition of p/q based on the amino acid sequence of a naturally occurring fibroin, and chemically synthesizing a nucleic acid encoding the designed amino acid sequence. In any case, in addition to the modification that corresponds substitution of one or a plurality of amino acid residues in REP with amino acid residues having a high hydropathy index and/or insertion of one or a plurality of amino acid residues having a high hydropathy index, as compared with a naturally occurring fibroin, a modification that corresponds to substitution, deletion, insertion, and/or addition of one or a plurality of amino acid residues may further be performed.

The amino acid residue having a high hydropathy index is not particularly limited, but is preferably isoleucine (I), valine (V), leucine (L), phenylalanine (F), cysteine (C), methionine (M), and alanine (A), and more preferably valine (V), leucine (L), and isoleucine (I).

More specific examples of the fifth modified fibroin contains a modified fibroin containing an amino acid sequence having (5-i) the amino acid sequence set forth in SEQ ID NO: 19 (Met-PRT720), SEQ ID NO: 20 (Met-PRT665), or SEQ ID NO: 21 (Met-PRT666), or (5-ii) an amino acid sequence having 90% or more sequence identity with the amino acid sequence set forth in SEQ ID NO: 19, SEQ ID NO: 20, or SEQ ID NO: 21.

The modified fibroin (5-i) will be described. The amino acid sequence set forth in SEQ ID NO: 19 is an amino acid sequence in which two amino acid sequences, each consisting of three amino acid residues (VLI), are inserted for every other REP excluding the terminal domain sequence on the C-terminal side; further, some of the glutamine (Q) residues are substituted with serine (S) residues; and some of the amino acids on the C-terminal side are deleted, with respect to the amino acid sequence set forth in SEQ ID NO: 7 (Met-PRT410). The amino acid sequence set forth in SEQ ID NO: 20 is an amino acid sequence in which one amino acid sequence consisting of three amino acid residues (VLI) is inserted for every other REP with respect to the amino acid sequence set forth in SEQ ID NO: 8 (Met-PRT525). The amino acid sequence set forth in SEQ ID NO: 21 is an amino acid sequence in which two amino acid sequences each consisting of three amino acid residues (VLI) are inserted for every other REP with respect to the amino acid sequence set forth in SEQ ID NO: 8.

The modified fibroin (5-i) may be a modified fibroin consisting of the amino acid sequence set forth in SEQ ID NO: 19, SEQ ID NO: 20, or SEQ ID NO: 21.

The modified fibroin (5-ii) is a modified fibroin containing an amino acid sequence having 90% or more sequence identity with the amino acid sequence set forth in SEQ ID NO: 19, SEQ ID NO: 20, or SEQ ID NO: 21. The modified fibroin (5-ii) also is a protein containing a domain sequence represented by Formula 1: [(A)ₙ motif-REP]ₘ. The sequence identity is preferably 95% or more.

It is preferable that the modified fibroin (5-ii) has 90% or more sequence identity with the amino acid sequence set forth in SEQ ID NO: 19, SEQ ID NO: 20, or SEQ ID NO: 21, and that p/q is 6.2% or more, where p is the total number of amino acid residues contained in a region where an average value of hydropathy indices of the four consecutive amino acid residues is 2.6 or more in all REPs contained in the domain sequence excluding the sequence between the (A)ₙ motif positioned closest to the C-terminal side and the C-terminal of the domain sequence; and where q is the total number of amino acid residues contained in the domain sequence excluding the sequence between the (A)ₙ motif positioned closest to the C-terminal side and the C-terminal of the domain sequence.

The fifth modified fibroin may contain a tag sequence at either or both of the N-terminal and C-terminal.

More specific examples of the modified fibroin containing a tag sequence include a modified fibroin containing (5-iii) the amino acid sequence set forth in SEQ ID NO: 22 (PRT720), SEQ ID NO: 23 (PRT665), or SEQ ID NO: 24 (PRT666), or (5-iv) an amino acid sequence having 90% or more sequence identity with the amino acid sequence set forth in SEQ ID NO: 22, SEQ ID NO: 23, or SEQ ID NO: 24.

The amino acid sequences set forth in SEQ ID NO: 22, SEQ ID NO: 23, and SEQ ID NO: 24 are amino acid sequences in which the amino acid sequence (containing a His tag sequence and a hinge sequence) set forth in SEQ ID NO: 11 is added to the N-terminal of the amino acid sequences set forth in SEQ ID NO: 19, SEQ ID NO: 20, and SEQ ID NO: 21, respectively.

The modified fibroin (5-iii) may be a modified fibroin consisting of the amino acid sequence set forth in SEQ ID NO: 22, SEQ ID NO: 23, or SEQ ID NO: 24.

The modified fibroin (5-iv) is a modified fibroin containing an amino acid sequence having 90% or more sequence identity with the amino acid sequence set forth in SEQ ID NO: 22, SEQ ID NO: 23, or SEQ ID NO: 24. The modified fibroin (5-iv) also is a protein containing a domain sequence represented by Formula 1: [(A)ₙ motif-REP]ₘ. The sequence identity is preferably 95% or more.

It is preferable that the modified fibroin (5-iv) has 90% or more sequence identity with the amino acid sequence set forth in SEQ ID NO: 22, SEQ ID NO: 23, or SEQ ID NO: 24, and that p/q is 6.2% or more, where p is the total number of amino acid residues contained in a region where an average value of hydropathy indices of the four consecutive amino acid residues is 2.6 or more in all REPs contained in the domain sequence excluding the sequence between the (A)ₙ motif positioned closest to the C-terminal side and the C-terminal of the domain sequence; and where q is the total number of amino acid residues contained in the domain sequence excluding the sequence between the (A)ₙ motif positioned closest to the C-terminal side and the C-terminal of the domain sequence.

The fifth modified fibroin may contain a secretory signal for releasing the protein produced in a recombinant protein production system to the outside of the host. The sequence of the secretory signal may be set as appropriate, depending on the type of the host.

The sixth modified fibroin has an amino acid sequence with a reduced content of glutamine residues, as compared with a naturally occurring fibroin.

It is preferable that the sixth modified fibroin contains at least one motif selected from a GGX motif and a GPGXX motif in the amino acid sequence of REP.

In a case where the sixth modified fibroin contains a GPGXX motif in REP, a GPGXX motif content rate is usually 1% or more, may be 5% or more, and is preferably 10% or more. The upper limit of the GPGXX motif content rate is not particularly limited, and it may be 50% or less, and may be 30% or less.

In the present specification, the “GPGXX motif content rate” is a value calculated by the following method.

In a fibroin (a modified fibroin or a naturally occurring fibroin) containing a domain sequence represented by Formula 1: [(A)ₙ motif-REP]ₘ or Formula 2: [(A)ₙ motif-REP]ₘ-(A)ₙ motif, the GPGXX motif content rate is calculated as s/t, where s is the number obtained by tripling the total number of the GPGXX motifs (namely, corresponds to the total number of G and P in the GPGXX motifs) contained in all REPs contained in the domain sequence excluding the sequence between the (A)ₙ motif positioned closest to the C-terminal side and the C-terminal of the domain sequence; and where t is the total number of amino acid residues in all REPs obtained by excluding the sequence between the (A)ₙ motif positioned closest to the C-terminal side and the C-terminal of the domain sequence from the domain sequence and further excluding the (A)ₙ motifs.

For the calculation of the GPGXX motif content rate, a “domain sequence excluding the sequence between the (A)ₙ motif positioned closest to the C-terminal side and the C-terminal of the domain sequence” is used, because “the sequence between the (A)ₙ motif positioned closest to the C-terminal side and the C-terminal of the domain sequence (sequence corresponding to REP) may contain a sequence having a low correlation with the sequence characteristic of a fibroin, which influences the calculation result of the GPGXX motif content rate when m is small (that is, when the domain sequence is short), and thus, this effect is to be eliminated. In a case where a “GPGXX motif” is located at the C-terminal of REP, it is treated as “GPGXX motif” even if “XX” is, for example, “AA”.

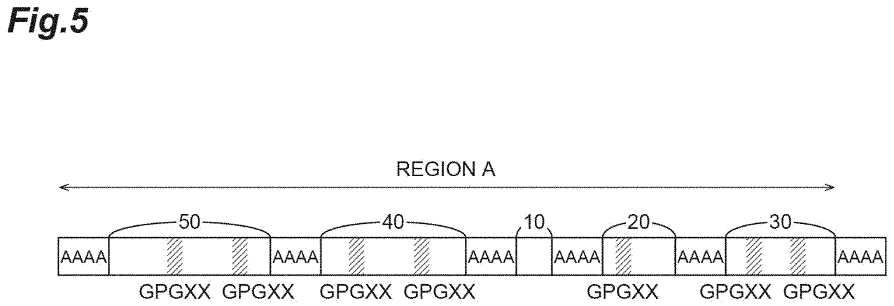

FIG. 5 is a schematic diagram illustrating a domain sequence of a modified fibroin. A method for calculating the GPGXX motif content rate will be specifically described with reference to FIG. 5. First, in a domain sequence of the modified fibroin (which is an [(A)ₙ motif-REP]ₘ-(A)ₙ motif] type) illustrated in FIG. 5, the number of GPGXX motifs for calculation of s is 7 and s is 7×3=21, because all REPs are contained in the “domain sequence excluding the sequence between the (A)ₙ motif positioned closest to the C-terminal side and the C-terminal of the domain sequence” (indicated as “REGION A” in FIG. 5). Similarly, since all REPs are contained in the “domain sequence excluding the sequence between the (A)ₙ motif positioned closest to the C-terminal side and the C-terminal of the domain sequence” (indicated as “REGION A” in FIG. 5), the total number, t, of amino acid residues in all REPs obtained by further excluding the (A)ₙ motifs from the “domain sequence excluding the sequence between the (A)ₙ motif positioned closest to the C-terminal side and the C-terminal of the domain sequence” is 50+40+10+20+30=150. Next, s/t (%) can be calculated by dividing s by t, and thus, in a case of the modified fibroin of FIG. 5, s/t (%) is 21/150=14.0%.

In the sixth modified fibroin, the glutamine residue content rate is preferably 9% or less, more preferably 7% or less, still more preferably 4% or less, and particularly preferably 0%.

In the present specification, the “glutamine residue content rate” is a value calculated by the following method.

In a fibroin (a modified fibroin or a naturally occurring fibroin) containing a domain sequence represented by Formula 1: [(A)ₙ motif-REP]ₘ or Formula 2: [(A)ₙ motif-REP]ₘ-(A)ₙ motif, the glutamine residue content rate is calculated as u/t, where u is the total number of glutamine residues contained in all REPs contained in the domain excluding the sequence between the (A)ₙ motif positioned closest to the C-terminal side and the C-terminal of the domain sequence (sequence corresponding to the “REGION A” in FIG. 5); and where t is the total number of amino acid residues in all REPs obtained by excluding the sequence between the (A)ₙ motif positioned closest to the C-terminal side and the C-terminal of the domain sequence from the domain sequence and further excluding the (A)ₙ motifs. The reason as to why the “domain sequence excluding the sequence between the (A)ₙ motif positioned closest to the C-terminal side and the C-terminal of the domain sequence” is used for the calculation of the glutamine residue content rate is the same as the reason described above.

The domain sequence of the sixth modified fibroin may be a domain sequence having an amino acid sequence that corresponds to an amino acid sequence in which one or a plurality of glutamine residues in REP are deleted or substituted with other amino acid residues, as compared with a naturally occurring fibroin.

The “other amino acid residues” may be amino acid residues other than the glutamine residue, but are preferably amino acid residues having a higher hydropathy index than that of the glutamine residue. The hydropathy indices of the amino acid residues are as shown in Table 1.

As shown in Table 1, examples of the amino acid residues having a higher hydropathy index than a glutamine residue include an amino acid residue selected from isoleucine (I), valine (V), leucine (L), phenylalanine (F), cysteine (C), methionine (M), alanine (A), glycine (G), threonine (T), serine (S), tryptophan (W), tyrosine (Y), proline (P), and histidine (H). Among these, an amino acid residue selected from isoleucine (I), valine (V), leucine (L), phenylalanine (F), cysteine (C), methionine (M), and alanine (A) is more preferable, and an amino acid residue selected from isoleucine (I), valine (V), leucine (L), and phenylalanine (F) is still more preferable.

In the sixth modified fibroin, the degree of hydrophobicity of REP is preferably −0.8 or more, more preferably −0.7 or more, still more preferably 0 or more, even still more preferably 0.3 or more, and particularly preferably 0.4 or more. The upper limit of the degree of hydrophobicity of REP is not particularly limited, and it may be 1.0 or less, and may be 0.7 or less.

In the present specification, the “degree of hydrophobicity of REP” is a value calculated by the following method.

In a fibroin (a modified fibroin or a naturally occurring fibroin) containing a domain sequence represented by Formula 1: [(A)ₙ motif-REP]ₘ or Formula 2: [(A)ₙ motif-REP]ₘ-(A)ₙ motif, the degree of hydrophobicity of REP is calculated as v/t, where v is the total sum of the hydropathy index of each amino acid residue contained in all REPs contained in the domain a sequence excluding the sequence between the (A)ₙ motif positioned closest to the C-terminal side and the C-terminal of the domain sequence (sequence corresponding to a “REGION A” in FIG. 5); and where t is the total number of amino acid residues in all REPs obtained by excluding the sequence between the (A)ₙ motif positioned closest to the C-terminal side and the C-terminal of the domain sequence from the domain sequence and further excluding the (A)ₙ motifs. The reason as to why the “domain sequence excluding the sequence between the (A)ₙ motif positioned closest to the C-terminal side and the C-terminal of the domain sequence” is used for the calculation of the degree of hydrophobicity of REP is the same as the reason described above.

The domain sequence of the sixth modified fibroin may further have a modification of an amino acid sequence that corresponds to substitution, deletion, insertion, and/or addition of one or a plurality of amino acid residues, in addition to the modification that corresponds to deletion of one or a plurality of glutamine residues in REP and/or substitution of one or a plurality of glutamine residues in REP with other amino acid residues, as compared with a naturally occurring fibroin.

The sixth modified fibroin may be obtained, for example, from a cloned gene sequence of a naturally occurring fibroin, by deleting one or a plurality of glutamine residues in REP and/or substituting one or a plurality of glutamine residues in REP with other amino acid residues.

In addition, the sixth modified fibroin may also be obtained by designing an amino acid sequence that corresponds to an amino acid sequence in which one or a plurality of glutamine residues in REP are deleted, and/or one or a plurality of glutamine residues in REP are substituted with other amino acid residues, based on the amino acid sequence of a naturally occurring fibroin, and chemically synthesizing a nucleic acid encoding the designed amino acid sequence.

More specific examples of the sixth modified fibroin include (6-i) a modified fibroin containing the amino acid sequence set forth in SEQ ID NO: 25 (Met-PRT888), SEQ ID NO: 26 (Met-PRT965), SEQ ID NO: 27 (Met-PRT889), SEQ ID NO: 28 (Met-PRT916), SEQ ID NO: 29 (Met-PRT918), SEQ ID NO: 30 (Met-PRT699), SEQ ID NO: 31 (Met-PRT698), SEQ ID NO: 32 (Met-PRT966), SEQ ID NO: 41 (Met-PRT917), or SEQ ID NO: 42 (Met-PRT1028), or (6-ii) a modified fibroin containing an amino acid sequence having 90% or more sequence identity with the amino acid sequence set forth in SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 41, or SEQ ID NO: 42.

The modified fibroin (6-i) will be described. The amino acid sequence set forth in SEQ ID NO: 25 is obtained by substituting all QQs in an amino acid sequence (Met-PRT410) set forth in SEQ ID NO: 7 with VLs. The amino acid sequence set forth in SEQ ID NO: 26 is obtained by substituting all QQs in the amino acid sequence set forth in SEQ ID NO: 7 with TSs, and substituting the remaining Qs with As. The amino acid sequence set forth in SEQ ID NO: 27 is obtained by substituting all QQs in the amino acid sequence set forth in SEQ ID NO: 7 with VLs, and substituting the remaining Qs with Is. The amino acid sequence set forth in SEQ ID NO: 28 is obtained by substituting all QQs in the amino acid sequence set forth in SEQ ID NO: 7 with VIs, and substituting the remaining Qs with Ls. The amino acid sequence set forth in SEQ ID NO: 29 is obtained by substituting all QQs in the amino acid sequence set forth in SEQ ID NO: 7 with VFs, and substituting the remaining Qs with Is.

The amino acid sequence set forth in SEQ ID NO: 30 is obtained by substituting all QQs in the amino acid sequence (Met-PRT525) set forth in SEQ ID NO: 8 with VLs. The amino acid sequence set forth in SEQ ID NO: 31 is obtained by substituting all QQs in the amino acid sequence set forth in SEQ ID NO: 8 with VLs, and substituting the remaining Qs with Is.

The amino acid sequence set forth in SEQ ID NO: 32 is a sequence obtained by substituting all QQs in a sequence obtained by repeating twice a region of 20 domain sequences present in the amino acid sequence (Met-PRT410) set forth in SEQ ID NO: 7 with VFs, and substituting the remaining Qs with Is.

The amino acid sequence (Met-PRT917) set forth in SEQ ID NO: 41 is an amino acid sequence in which all QQs are substituted with LIs and the remaining Qs are substituted with Vs in the amino acid sequence set forth in SEQ ID NO: 7. The amino acid sequence (Met-PRT1028) set forth in SEQ ID NO: 42 is an amino acid sequence in which all QQs are substituted with IFs and the remaining Qs are substituted with Ts in the amino acid sequence set forth in SEQ ID NO: 7.

The glutamine residue content rate of any of the amino acid sequences set forth in SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 41, and SEQ ID NO: 42 is 9% or less (Table 2).

TABLE 2

| | Glutamine | GPGXX | |
|---|---|---|---|
| | residue | motif | Degree of |
| | content | content | hydrophobicity |
| Modified fibroin | rate | rate | of REP |
| Met-PRT410 (SEQ ID NO: 7) | 17.7% | 27.9% | −1.52 |
| Met-PRT888 (SEQ ID NO: 25) | 6.3% | 27.9% | −0.07 |
| Met-PRT965 (SEQ ID NO: 26) | 0.0% | 27.9% | −0.65 |
| Met-PRT889 (SEQ ID NO: 27) | 0.0% | 27.9% | 0.35 |
| Met-PRT916 (SEQ ID NO: 28) | 0.0% | 27.9% | 0.47 |
| Met-PRT918 (SEQ ID NO: 29) | 0.0% | 27.9% | 0.45 |
| Met-PRT699 (SEQ ID NO: 30) | 3.6% | 26.4% | −0.78 |
| Met-PRT698 (SEQ ID NO: 31) | 0.0% | 26.4% | −0.03 |
| Met-PRT966 (SEQ ID NO: 32) | 0.0% | 28.0% | 0.35 |
| Met-PRT917 (SEQ ID NO: 41) | 0.0% | 27.9% | 0.46 |
| Met-PRT1028 (SEQ ID NO: 42) | 0.0% | 28.1% | 0.05 |

The modified fibroin (6-i) may be a modified fibroin consisting of the amino acid sequence set forth in SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 41, or SEQ ID NO: 42.

The modified fibroin (6-ii) may be a modified fibroin containing an amino acid sequence having 90% or more sequence identity with the amino acid sequence set forth in SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 41, or SEQ ID NO: 42. The modified fibroin (6-ii) also is a protein containing a domain sequence represented by Formula 1: [(A)ₙ motif-REP]ₘ or Formula 2: [(A)ₙ motif-REP]ₘ-(A)ₙ motif. The sequence identity is preferably 95% or more.

The modified fibroin (6-ii) preferably has a glutamine residue content rate of 9% or less. In addition, the modified fibroin (6-ii) preferably has a GPGXX motif content rate of 10% or more.

The sixth modified fibroin may contain a tag sequence at either or both of the N-terminal and C-terminal. This makes it possible to isolate, immobilize, detect, and visualize the modified fibroin.

More specific examples of the modified fibroin containing a tag sequence include a modified fibroin containing (6-iii) the amino acid sequence set forth in SEQ ID NO: 33 (PRT888), SEQ ID NO: 34 (PRT965), SEQ ID NO: 35 (PRT889), SEQ ID NO: 36 (PRT916), SEQ ID NO: 37. (PRT918), SEQ ID NO: 38 (PRT699), SEQ ID NO: 39 (PRT698), SEQ ID NO: 40 (PRT966), SEQ ID NO: 43 (PRT917), or SEQ ID NO: 44 (PRT1028), or a modified fibroin containing (6-iv) an amino acid sequence having 90% or more sequence identity with the amino acid sequence set forth in SEQ ID NO: 33, SEQ ID NO: 34, SEQ ID NO: 35, SEQ ID NO: 36, SEQ ID NO: 37, SEQ ID NO: 38, SEQ ID NO: 39 or SEQ ID NO: 40, SEQ ID NO: 43, or SEQ ID NO: 44.

The amino acid sequences set forth in SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, SEQ ID NO: 43, and SEQ ID NO: 44 are amino acid sequences in which the amino acid sequence (containing a His tag sequence and a hinge sequence) set forth in SEQ ID NO: 11 is added to the N-terminal of the amino acid sequences set forth in SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 41, and SEQ ID NO: 42, respectively. Since only the tag sequence is added to the N-terminal, the glutamine residue content rate is not changed, and any of the amino acid sequences set forth in SEQ ID NO:33, SEQ ID NO:34, SEQ ID NO:35, SEQ ID NO:36, SEQ ID NO:37, SEQ ID NO:38, SEQ ID NO:39, SEQ ID NO: 40, SEQ ID NO: 43, and SEQ ID NO: 44 has a glutamine residue content rate of 9% or less (Table 3).

TABLE 3

| | Glutamine | GPGXX | |
|---|---|---|---|
| | residue | motif | Degree of |
| | content | content | hydrophobicity |
| Modified fibroin | rate | rate | of REP |
| PRT888 (SEQ ID NO: 33) | 6.3% | 27.9% | −0.07 |
| PRT965 (SEQ ID NO: 34) | 0.0% | 27.9% | −0.65 |
| PRT889 (SEQ ID NO: 35) | 0.0% | 27.9% | 0.35 |
| PRT916 (SEQ ID NO: 36) | 0.0% | 27.9% | 0.47 |
| PRT918 (SEQ ID NO: 37) | 0.0% | 27.9% | 0.45 |
| PRT699 (SEQ ID NO: 38) | 3.6% | 26.4% | −0.78 |
| PRT698 (SEQ ID NO: 39) | 0.0% | 26.4% | −0.03 |
| PRT966 (SEQ ID NO: 40) | 0.0% | 28.0% | 0.35 |
| PRT917 (SEQ ID NO: 43) | 0.0% | 27.9% | 0.46 |
| PRT1028 (SEQ ID NO: 44) | 0.0% | 28.1% | 0.05 |

The modified fibroin (6-iii) may be a modified fibroin consisting of the amino acid sequence set forth in SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, SEQ ID NO: 43, or SEQ ID NO: 44.

The modified fibroin (6-iv) is a modified fibroin containing an amino acid sequence having 90% or more sequence identity with the amino acid sequence set forth in SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, SEQ ID NO: 43, or SEQ ID NO: 44. The modified fibroin (6-iv) also is a protein containing a domain sequence represented by Formula 1: [(A)ₙ motif-REP]ₘ or Formula 2: [(A)ₙ motif-REP]ₘ-(A)ₙ motif. The sequence identity is preferably 95% or more.

The modified fibroin (6-iv) preferably has a glutamine residue content rate of 9% or less. In addition, the modified fibroin (6-iv) preferably has a GPGXX motif content rate of 10% or more.

The sixth modified fibroin may contain a secretory signal for releasing the protein produced in a recombinant protein production system to the outside of the host. The sequence of the secretory signal may be set as appropriate, depending on the type of the host.

The modified fibroin may be a modified fibroin simultaneously pertaining at least two or more characteristics among the characteristics of the first modified fibroin, the second modified fibroin, the third modified fibroin, the fourth modified fibroin, the fifth modified fibroin, and the sixth modified fibroin.

(Method for Manufacturing Modified Fibroin)

The modified fibroin (hereinafter also simply referred to as a “protein”) according to any of the embodiments may also be produced, for example, by expressing a nucleic acid by a host transformed with an expression vector having the nucleic acid sequence encoding the protein and one or a plurality of regulatory sequences operably linked to the nucleic acid sequence.

A method for producing a nucleic acid encoding a modified fibroin is not particularly limited. For example, the nucleic acid may be produced by a method in which a gene encoding a natural fibroin is cloned by amplification with a polymerase chain reaction (PCR) or the like, and subjected to a modification by a genetic engineering technique, or a method of chemically synthesizing a nucleic acid. The method for chemically synthesizing a nucleic acid is not particularly limited, and for example, a gene may be chemically synthesized by a method in which oligonucleotides are automatically synthesized by AKTA oligopilot plus 10/100 (GE Healthcare Japan Corporation) or the like, and are linked by PCR or the like, based on the amino acid sequence information of a protein obtained from the NCBI web database or the like. At this time, in order to facilitate purification and/or confirmation of the modified fibroin, a nucleic acid encoding a modified fibroin consisting of an amino acid sequence obtained by adding an amino acid sequence consisting of a start codon and a His10 tag to the N-terminal of the above-described amino acid sequence may be synthesized.

The regulatory sequence is a sequence (for example, a promoter, an enhancer, a ribosome binding sequence, or a transcription termination sequence) that controls the expression of a modified fibroin in a host, and may be selected as appropriate, depending on the type of the host. As a promoter, an inducible promoter that functions in a host cell and is capable of inducing the expression of a modified fibroin may be used. The inducible promoter is a promoter that can control transcription by the presence of an inducer (expression inducer), the absence of a repressor molecule, or physical factors such as an increase or decrease in a temperature, an osmotic pressure, or a pH value.

The type of the expression vector such as a plasmid vector, a viral vector, a cosmid vector, a fosmid vector, or an artificial chromosome vector may be selected as appropriate, depending on the type of the host. As the expression vector, an expression vector that can autonomously replicate in a host cell or can be incorporated into a chromosome of a host and which contains a promoter at a position capable of transcribing the nucleic acid that encodes a modified fibroin is suitably used.

Both prokaryotes and eukaryotes such as yeast, filamentous fungi, insect cells, animal cells, and plant cells may be suitably used as a host.

Preferred examples of the prokaryotic host cells include bacteria belonging to the genus Escherichia, the genus Brevibacillus, the genus Serratia, the genus Bacillus, the genus Microbacterium, the genus Brevibacterium, the genus Corynebacterium, and the genus Pseudomonas. Examples of microorganisms belonging to the genus Escherichia include Escherichia coli. Examples of microorganisms belonging to the genus Brevibacillus include Brevibacillus agri. Examples of microorganisms belonging to the genus Serratia include Serratia liquefaciens. Examples of microorganisms belonging to the genus Bacillus include Bacillus subtilis. Examples of microorganisms belonging to the genus Microbacterium include Microbacterium ammoniaphilum. Examples of microorganisms belonging to the genus Brevibacterium include Brevibacterium divaricatum. Examples of microorganisms belonging to the genus Corynebacterium include Corynebacterium ammoniagenes. Examples of microorganisms belonging to the genus Pseudomonas include Pseudomonas putida.

In a case where a prokaryote is used as a host, examples of a vector into which a nucleic acid encoding a modified fibroin is introduced include pBTrp2 (manufactured by Boehringer Mannheim), pGEX (manufactured by Pharmacia), pUC18, pBluescriptII, pSupex, pET22b, pCold, pUB110, and pNCO2 (Japanese Unexamined Patent Publication No. 2002-238569).

Examples of the eukaryotic hosts include yeast and filamentous fungi (mold and the like). Examples of the yeasts include yeasts belonging to the genus Saccharomyces, the genus Pichia, and the genus Schizosaccharomyces. Examples of the filamentous fungi include filamentous fungi belonging to the genus Aspergillus, the genus Penicillium, and the genus Trichoderma.

In a case where the eukaryote is used as a host, examples of the vector into which a nucleic acid encoding a modified fibroin is introduced include YEp13 (ATCC37115) and YEp24 (ATCC37051). As a method for introducing an expression vector into the host cell, any of methods of introducing DNA into the host cell may be used. Examples thereof include a method using calcium ions [Proc. Natl. Acad. Sci. USA, 69, 2110 (1972)], electroporation method, spheroplast method, protoplast method, lithium acetate method, and competent method.

As for the method for expressing a nucleic acid using a host transformed with an expression vector, secretory production, fusion protein expression, or the like, in addition to the direct expression, may be performed in accordance with the method described in Molecular Cloning, 2^{nd} edition, and the like.

The modified fibroin may be produced, for example, by culturing a host transformed with the expression vector in a culture medium, producing and accumulating the protein in the culture medium, and then collecting the modified fibroin from the culture medium. The method for culturing a host in a culture medium may be performed in accordance with a method commonly used for culturing a host.

In a case where the host is a prokaryote such as Escherichia coli or a eukaryote such as yeast, any of a natural medium and a synthetic medium may be used as a culture medium of the host as long as the medium contains a carbon source, a nitrogen source, inorganic salts, and the like which may be utilized by the host and the medium may be used for efficiently culturing the host.

The carbon source may be any of carbon sources which can be assimilated by a transformed microorganism, and for example, carbohydrates such as glucose, fructose, sucrose, and molasses, starch, and starch hydrolysates containing these, organic acids such as acetic acid and propionic acid, and alcohols such as ethanol and propanol may be used. As the nitrogen source, for example, ammonium salts of inorganic or organic acids such as ammonia, ammonium chloride, ammonium sulfate, ammonium acetate, and ammonium phosphate, other nitrogen-containing compounds, peptone, meat extract, yeast extract, corn steep liquor, casein hydrolysate, soybean cake and soybean cake hydrolysate, and various fermented microbial cells and digested products thereof may be used. As the inorganic salts, for example, potassium dihydrogen phosphate, dipotassium phosphate, magnesium phosphate, magnesium sulfate, sodium chloride, ferrous sulfate, manganese sulfate, copper sulfate, and calcium carbonate may be used.

Culture of a prokaryote such as Escherichia coli or a eukaryote such as a yeast may be performed under aerobic conditions such as shaking culture or deep aeration stirring culture. The culture temperature is, for example, 15° C. to 40° C. The culture time is usually 16 hours to 7 days. It is preferable to maintain the pH of the culture medium during the culture at 3.0 to 9.0. The pH of the culture medium may be adjusted using an inorganic acid, an organic acid, an alkali solution, urea, calcium carbonate, ammonia, or the like.

In addition, antibiotics such as ampicillin and tetracycline may be added to the culture medium as necessary during the culture. In a case of culturing a microorganism transformed with an expression vector using an inducible promoter as the promoter, an inducer may be added to the medium as necessary. For example, in a case of culturing a microorganism transformed with an expression vector using a lac promoter, isopropyl-β-D-thiogalactopyranoside or the like may be added to the medium, and in a case of culturing a microorganism transformed with an expression vector using a trp promoter, indole acrylic acid or the like may be added to the medium.

Isolation and purification of the expressed modified fibroin may be performed by a commonly used method. For example, in a case where the modified fibroin is expressed in a dissolved state in cells, the host cells are recovered by centrifugation after the completion of the culture, suspended in an aqueous buffer solution, and then disrupted using an ultrasonicator, a French press, a Manton-Gaulin homogenizer, a Dyno-Mill, or the like to obtain a cell-free extract. From the supernatant obtained by centrifuging the cell-free extract, a purified preparation may be obtained by a method commonly used for protein isolation and purification, that is, a solvent extraction method, a salting-out method using ammonium sulfate or the like, a desalting method, a precipitation method using an organic solvent, an anion exchange chromatography method using a resin such as diethylaminoethyl (DEAE)-Sepharose or DIAION HPA-75 (manufactured by Mitsubishi Kasei Kogyo Kabushiki Kaisha), an cation exchange chromatography method using a resin such as S-Sepharose FF (manufactured by Pharmacia Corporation), a hydrophobic chromatography method using a resin such as butyl sepharose or phenyl sepharose, a gel filtration method using a molecular sieve, an affinity chromatography method, a chromatofocusing method, or an electrophoresis method such as isoelectric focusing or the like, using the above-mentioned methods singly or in combination thereof.

In addition, in a case where the modified fibroin is expressed to form an insoluble body in the cell, similarly, the host cells are recovered, disrupted, and centrifuged to recover the insoluble body of the modified fibroin as a precipitated fraction. The recovered insoluble body of the modified fibroin may be solubilized with a protein denaturing agent. After this operation, a purified preparation of the modified fibroin may be obtained by the same isolation and purification method as described above. In a case where the protein is secreted extracellularly, the modified fibroin may be recovered from the culture supernatant. That is, a culture supernatant is obtained by treating the culture by a technique such as centrifugation, and a purified preparation may be obtained from the culture supernatant by using the same isolation and purification method as described above.

(Raw Material Fiber)

A raw material fiber according to the embodiment is obtained by spinning the above-described modified fibroin, and includes the above-described modified fibroin as a main component. The raw material fiber according to the embodiment is a fiber after spinning and before contact with water.

(Method of Manufacturing Raw Material Fiber)

The raw material fiber according to the embodiment can be manufactured by a known spinning method. That is, for example, when a raw material fiber including a modified fibroin as a main component is manufactured, first, the modified fibroin manufactured according to the above-described method is added to and dissolved in a solvent such as dimethyl sulfoxide (DMSO), N,N-dimethylformamide (DMF), a formic acid, or hexafluoroisopropanol (HFIP), if necessary, together with an inorganic salt as a dissolution accelerator to prepare a dope solution (a spinning dope solution). Then, spinning by a known spinning method such as wet spinning, dry spinning, dry-wet spinning, or melt spinning is performed using the dope solution, and a desired raw material fiber can be obtained. Preferably, the spinning method is wet spinning or dry-wet spinning.

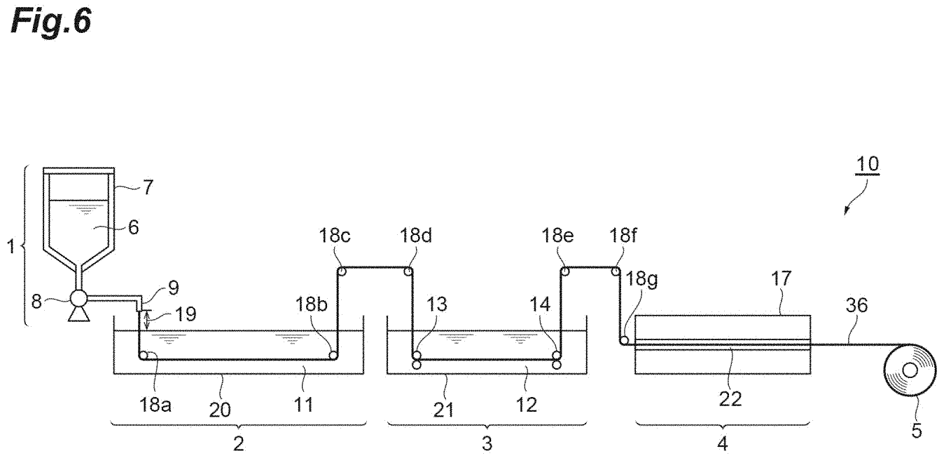

FIG. 6 is an explanatory diagram schematically showing an example of a spinning apparatus for producing a raw material fiber. A spinning apparatus 10 shown in FIG. 6 is an example of a spinning apparatus for dry-wet spinning and includes an extrusion device 1, an undrawn yarn manufacturing device 2, a wet heat drawing device 3, and a drying device 4.

A spinning method using the spinning apparatus 10 will be described. First, a dope solution 6 stored in a storage tank 7 is extruded from a tap 9 by a gear pump 8. In a laboratory scale, the dope solution may be filled into a cylinder and then may be extruded from a nozzle using a syringe pump. Next, the extruded dope solution 6 is supplied into a coagulating solution 11 of a coagulating solution tank 20 through an air gap 19, the solvent is removed, the modified fibroin is coagulated, and a fibrous coagulated body is formed. Next, the fibrous coagulated body is supplied and drawn into hot water 12 in a drawing bath 21. A drawing ratio is determined by a speed ratio between a supply nip roller 13 and a take-up nip roller 14. After that, the drawn fibrous coagulated body is supplied to the drying device 4 and dried in a yarn path 22, and a raw material fiber is obtained as a wound yarn body 5. 18a to 18g are thread guides.

The coagulating solution 11 may be any solvent which can be desolvated, and examples thereof include acetone, lower alcohols having 1 to 5 carbon atoms such as methanol, ethanol and 2-propanol, and the like. The coagulating solution 11 may appropriately contain water. A temperature of the coagulating solution 11 is preferably 0 to 30° C. When a syringe pump having a nozzle with a diameter of 0.1 to 0.6 mm is used as the tap 9, an extrusion speed is preferably 0.2 to 6.0 ml/hour, and more preferably 1.4 to 4.0 ml/hour per hole. A distance through which a coagulated protein passes through the coagulating solution 11 (substantially, a distance from the thread guide 18a to the thread guide 18b) may be a length which allows efficient desolvation, and is, for example, 200 to 500 mm. A take-up speed of the undrawn yarn may be, for example, 1 to 20 m/min, and preferably 1 to 3 m/min A residence time in the coagulating solution 11 may be, for example, 0.01 to 3 minutes, and preferably 0.05 to 0.15 minutes. Moreover, drawing (pre-drawing) may be performed in the coagulating solution 11. The coagulating solution tank 20 may be provided in multiple stages, and the drawing may be performed in each of the stages or in a specific stage, if necessary.

For example, in addition to the pre-drawing performed in the coagulating solution tank 20 and the wet heat drawing performed in the drawing bath 21, dry heat drawing is also adopted as the drawing performed when the raw material fiber is obtained.

The wet heat drawing can be performed in warm water, in a solution in which an organic solvent or the like is added to the warm water, or in steam heating. A temperature thereof may be, for example, 50 to 90° C., and preferably 75 to 85° C. In the wet heat drawing, the undrawn yarn (or pre-drawn yarn) can be drawn, for example, 1 to 10 times, and preferably 2 to 8 times.

The dry heat drawing can be performed using an electric tubular furnace, a dry heat plate, or the like. A temperature thereof may be, for example, 140° C. to 270° C., and preferably 160° C. to 230° C. In the dry heat drawing, the undrawn yarn (or the pre-drawn yarn) can be drawn 0.5 to 8 times, for example, and preferably 1 to 4 times.

The wet heat drawing and the dry heat drawing may be performed individually, or may be performed in multiple stages or in combination. That is, the wet heat drawing and the dry heat drawing can be appropriately combined, such as performing the first stage drawing by the wet heat drawing and performing the second stage drawing by the dry heat drawing, or performing the first stage drawing by the wet heat drawing, performing the second stage drawing by the wet heat drawing and further performing the third stage drawing by the dry heat drawing, or the like.

The lower limit of the final drawing ratio is preferably any one of more than 1 time, 2 times or more, 3 times or more, 4 times or more, 5 times or more, 6 times or more, 7 times or more, 8 times or more, and 9 times or more with respect to the undrawn yarn (or the pre-drawn yarn), and the upper limit thereof is preferably 40 times or less, 30 times or less, 20 times or less, 15 times or less, 14 times or less, 13 times or less, 12 times or less, 11 times or less, or 10 times or less. When the raw material fiber is a fiber spun with a drawing ratio of 2 times or more, a contraction rate when the raw material fiber is brought into contact with water and is in a wet state becomes higher.

(Method of Manufacturing Artificial Hair Fiber (Artificial Fibroin Fiber))

The artificial hair fiber (the artificial fibroin fiber) according to the embodiment can be obtained by a manufacturing method including a contracting step of contracting the raw material fiber with water. The contracting step may include, for example, a step (a contact step) in which the above-described raw material fiber (the raw material fiber after spinning and before contact with water) is brought into contact with water and irreversibly contracts. The contracting step may include a step (a drying step) of drying the fiber and further contracting the fiber after the contact step.

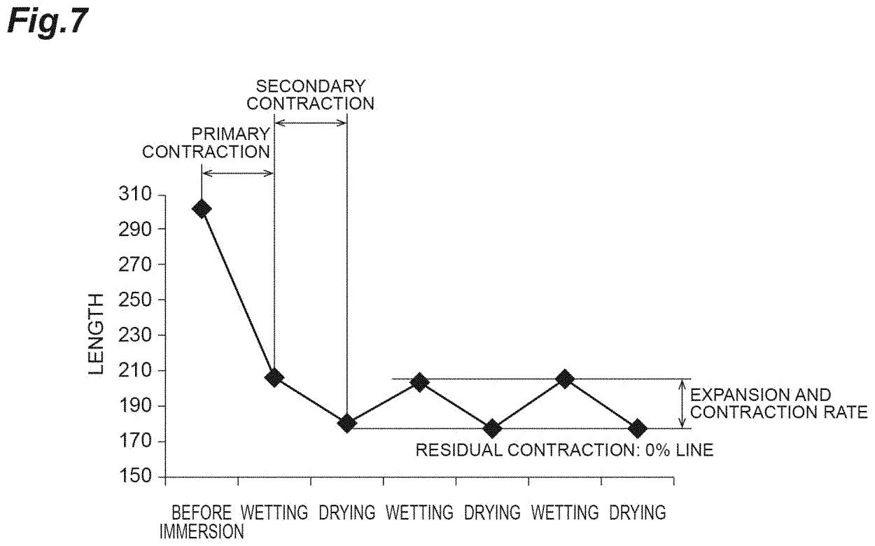

FIG. 7 is a diagram showing an example of a change in a length of the raw material fiber (the fiber containing the modified fibroin) due to contact with water. The raw material fiber (the fiber containing the modified fibroin) according to the embodiment has a property in which it contracts when it comes into contact with water (is wet) (a change in the length shown by a “primary contraction” in FIG. 7). After the primary contraction, when it is dried, it further contracts (a change in the length shown by a “secondary contraction” in FIG. 7). After the secondary contraction, when it is brought into contact with water again, it expands to the same or similar length as before the secondary contraction, and after that, when drying and wetting are repeated, the contraction and the expansion are repeated with the same width as that in the secondary contraction (a width indicated by a “expansion and contraction rate” in FIG. 7). Therefore, the artificial hair fiber according to the embodiment can be obtained by a manufacturing method which includes at least the contracting step including the contact step.

The irreversible contraction (the “primary contraction” in FIG. 7) of the raw material fiber (the fiber containing the modified fibroin) in the contact step is considered to occur, for example, for the following reasons. That is, one reason is considered to be due to a secondary structure or a tertiary structure of the raw material fiber (the fiber containing the modified fibroin), and another reason is considered to be, for example, due to residual stress being relaxed by infiltration of water between the fibers or into the fiber in the raw material fiber (the fiber containing the modified fibroin) having residual stress due to drawing or the like in the manufacturing process. Therefore, it is considered that the contraction rate of the raw material fiber (the fiber containing the modified fibroin) in the contracting step can be arbitrarily controlled, for example, according to a magnitude of the drawing ratio in the manufacturing process of the above-described raw material fiber (the fiber containing the modified fibroin).

In the contact step, the raw material fiber after spinning and before contact with water is brought into contact with water to bring the raw material fiber into the wet state. The wet state is a state in which at least a part of the raw material fiber is wet with water. Thus, the raw material fiber can be contracted without depending on an external force. This contraction is irreversible (corresponding to the “primary contraction” in FIG. 7).

A temperature of the water brought into contact with the raw material fiber in the contact step may be below a boiling point. Thus, handleability and workability of the contracting step are improved. Further, from the viewpoint of sufficiently shortening a contraction time, the lower limit of the temperature of the water is preferably 10° C. or higher, more preferably 40° C. or higher, and further preferably 70° C. or higher. The upper limit of the temperature of the water is preferably 90° C. or lower.

In the contact step, a method of bringing water into contact with the raw material fiber is not particularly limited. Examples of the method include a method of immersing the raw material fiber in water, a method of spraying the raw material fiber with water at room temperature or in a heated steam state, a method of exposing the raw material fiber in a high humidity environment filled with water vapor, and the like. In the contact step, among these methods, the method of immersing the raw material fiber in water is preferable because the contraction time can be effectively shortened and a processing facility can be simplified.

In the contact step, when the raw material fiber is brought into contact with water in a relaxed state, the raw material fiber may not only simply contract but also shrink in a wavy manner. In order to prevent occurrence of such shrinkage, the contact step may be carried out in a state in which the raw material fiber is not relaxed, for example, the raw material fiber is brought into contact with water while it is tensioned (pulled) in a fiber axis direction.

The method of manufacturing an artificial hair fiber (an artificial fibroin fiber) according to the embodiment may further include a drying step. The drying step is a step of drying the raw material fiber (or the artificial hair fiber obtained through the contact step) which have been subjected to the contact step and further contracting the raw material fiber (corresponding to the “secondary contraction” in FIG. 7). The drying may be, for example, natural drying, or forced drying using a drying facility. As the drying facility, any known contact type or non-contact type drying facility can be used. Further, although a drying temperature is not limited, for example, as long as it is lower than a temperature at which the protein contained in the raw material fiber is decomposed or the raw material fiber is thermally damaged, generally, the temperature is in a range of 20 to 150° C., and the temperature is preferably in a range of 50 to 100° C. The fiber is dried more quickly and efficiently without causing the thermal damage to the fiber or the decomposition of the protein contained in the fiber by keeping the temperature in this range. A drying time is appropriately set according to the drying temperature and the like. For example, a time during which an influence of overdrying on the quality and physical properties of the artificial hair fiber (the artificial fibroin fiber) can be eliminated as much as possible is adopted.

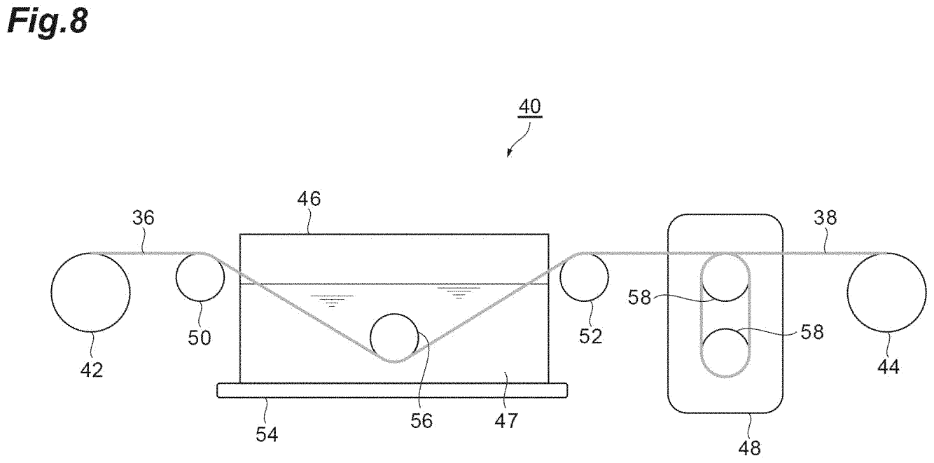

FIG. 8 is an explanatory diagram schematically showing an example of a manufacturing apparatus for manufacturing an artificial hair fiber (an artificial fibroin fiber). A manufacturing apparatus 40 shown in FIG. 8 includes a feed roller 42 which feeds out a raw material fiber, a winder 44 which winds up an artificial hair fiber 38, a water bath 46 which performs the contact step, and a dryer 48 which performs the drying step.

More specifically, the feed roller 42 is formed so that a wound material of a raw material fiber 36 can be mounted thereon, and the raw material fiber 36 can be continuously and automatically delivered from the wound material of the raw material fiber 36 by rotation of an electric motor or the like (not shown). The winder 44 can continuously and automatically wind up the artificial hair fiber 38 manufactured through the contact step and the drying step after it is delivered from the feed roller 42 by rotation of an electric motor (not shown). Here, a delivering speed of the raw material fiber 36 by the feed roller 42 and a winding speed of the artificial hair fiber 38 by the winder 44 can be controlled independently of each other.

The water bath 46 and the dryer 48 are arranged and disposed between the feed roller 42 and the winder 44 on the upstream side and the downstream side in a delivering direction of the raw material fiber 36. The manufacturing apparatus 40 shown in FIG. 8 includes relay rollers 50 and 52 which relay the raw material fiber 36 before and after the contact step which travels from the feed roller 42 toward the winder 44.

The water bath 46 includes a heater 54, and water 47 heated by the heater 54 is accommodated in the water bath 46. Further, in the water bath 46, a tension roller 56 is installed in a state in which it is immersed in the water 47. Thus, the raw material fiber 36 delivered from the feed roller 42 travels toward the winder 44 side while it is immersed in the water 47 in a state in which it is wound around the tension roller 56 in the water bath 46. An immersion time of the raw material fiber 36 in the water 47 is appropriately controlled according to a traveling speed of the raw material fiber 36.

The dryer 48 includes a pair of hot rollers 58. The pair of hot rollers 58 are formed so that the raw material fiber 36 which is separated from the water bath 46 and travels toward the winder 44 side can be wound thereon. Thus, the raw material fiber 36 immersed in the water 47 in the water bath 46 is heated by the pair of hot rollers 58 in the dryer 48, dried, and then further delivered toward the winder 44.

When the artificial hair fiber 38 is manufactured using the manufacturing apparatus 40 having such a structure, first, for example, the wound material of the raw material fiber 36 spun using the spinning apparatus 10 shown in FIG. 6 is mounted on the feed roller 42. Next, the raw material fiber 36 is continuously delivered from the feed roller 42 and is immersed in the water 47 in the water bath 46. At this time, for example, a winding speed of the winder 44 is set to be slower than the delivering speed of the feed roller 42. Thus, since the raw material fiber 36 contracts due to contact with the water 47 in a state in which it is tense between the feed roller 42 and the winder 44 not to relax, the occurrence of the shrinkage can be prevented. The raw material fiber 36 contracts irreversibly due to the contact with the water 47 (corresponding to the “primary contraction” in FIG. 7).

Next, the raw material fiber 36 after the contact with the water 47 (or the artificial hair fiber 38 manufactured through the contact with the water 47) is heated by the pair of hot rollers 58 of the dryer 48. Thus, the raw material fiber 36 after contact with water 47 (or the artificial hair fiber 38 manufactured through the contact with the water 47) can be dried and further contracted (corresponding to the “secondary contraction” in FIG. 7). At this time, the artificial hair fiber 38 can be further contracted or the length thereof can be kept unchanged by controlling a ratio between the delivering speed of the feed roller 42 and the winding speed of the winder 44. Then, the obtained artificial hair fiber 38 is wound by a winder 44, and the wound material of the artificial hair fiber 38 is obtained.

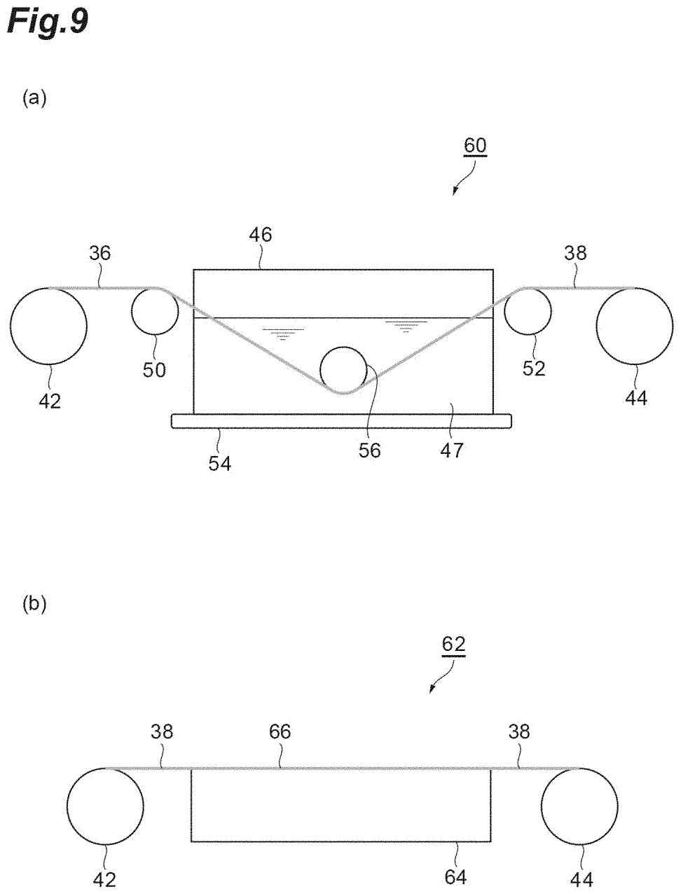

Instead of the pair of hot rollers 58, the raw material fiber 36 after the contact with the water 47 may be dried using a drying facility only including a simple heat source such as a dry heat plate 64 as shown in FIG. 9(b). Also in this case, the artificial hair fiber 38 can be further contracted, or the length thereof can be kept unchanged by adjusting a relative speed between the delivering speed of the feed roller 42 and the winding speed of the winder 44 in the same manner as when the pair of hot rollers 58 are used as the drying facility. Here, a drying means is configured of the dry heat plate 64. Moreover, the dryer 48 is not indispensable.

As described above, the target artificial hair fiber 38 can be manufactured automatically, continuously, and extremely easily using the manufacturing apparatus 40.

FIG. 9 is an explanatory diagram schematically showing another example of the manufacturing apparatus for manufacturing an artificial hair fiber (an artificial fibroin fiber). FIG. 9(a) shows a processing device provided in the manufacturing apparatus to perform the contact step, and FIG. 9(b) shows a drying device provided in the manufacturing apparatus to perform the drying step. The manufacturing apparatus shown in FIG. 9 includes a processing device 60 which performs a contact step with respect to the raw material fiber 36, and a drying device 62 which dries the raw material fiber 36 after the contact step (or the artificial hair fiber 38 manufactured through the contact step), and they have structures which are independent of each other.

More specifically, the processing device 60 shown in FIG. 9(a) has a structure in which the dryer 48 is omitted from the manufacturing apparatus 40 shown in FIG. 8, and the feed roller 42, the water bath 46, and the winder 44 are arranged and disposed in order from the upstream side to the downstream side in the traveling direction of the raw material fiber 36. Such a processing device 60 is formed to immerse the raw material fiber 36 delivered from the feed roller 42 in the water 47 in the water bath 46 and to contract the raw material fiber 36. Additionally, the processing device 60 is configured so that the obtained artificial hair fiber 38 is wound up by the winder 44.

The drying device 62 shown in FIG. 9(b) includes the feed roller 42, the winder 44, and the dry heat plate 64. The dry heat plate 64 is disposed between the feed roller 42 and the winder 44 so that a dry heat surface 66 comes into contact with the artificial hair fiber 38 and extends in the traveling direction thereof. In the drying device 62, the artificial hair fiber 38 can be further contracted or the length thereof can be kept unchanged, for example, by controlling the ratio between the delivering speed of the feed roller 42 and the winding speed of the winder 44 as described above.

The raw material fiber 36 can be contracted by the processing device 60 to obtain the artificial hair fiber 38 using the manufacturing apparatus having such a structure, and then the artificial hair fiber 38 can be dried by the drying device 62.

The feed roller 42 and the winder 44 may be omitted from the processing device 60 shown in FIG. 9(a), and the processing device may be configured only of the water bath 46. When a manufacturing apparatus having such a processing device is used, for example, the artificial hair fiber is manufactured in a so-called batch manner. Further, the drying device 62 shown in FIG. 9(b) is not indispensible.

(Artificial Hair Fibers (Artificial Fibroin Fibers))

Since the artificial hair fibers (the artificial fibroin fibers) according to the embodiment are obtained by, for example, the above-described manufacturing method, they expand when they are in a wet state and contract when they dry from a wet state (corresponding to expansion and contraction after the “secondary contraction” in FIG. 7). Since the artificial hair fiber (the artificial fibroin fiber) according to the embodiment is obtained by, for example, the above-described manufacturing method, it does not substantially contain residual stress generated by drawing in the spinning process.

The artificial hair fiber according to the embodiment may have a restoration rate of 95% or more as defined by the following Formula (1).

Restoration rate=(length of artificial fibroin fiber when dried from wet state/length of artificial fibroin fiber before wet state)×100(%) Formula (1):

As the restoration rate defined by Formula (1) becomes higher, the behavior during wetting and drying becomes closer to that of human hair, and thus it becomes possible to curb occurrence of a feeling of strangeness with artificial hair in comparison with human hair. In the artificial hair fiber according to the embodiment, the restoration rate defined by Formula (1) is preferably 96% or more, more preferably 97% or more, further preferably 98% or more, and even more preferably 99% or more.

The artificial hair fiber according to the embodiment may have an expansion rate of 17% or less as defined by the following Formula (4). The expansion rate defined by Formula (4) is an index of expansion characteristics when the artificial hair fiber is in the wet state.

Expansion rate={(length of artificial fibroin fiber in wet state/length of artificial fibroin fiber before wet state)−1}×100(%) Formula (4):

The artificial hair fiber according to the embodiment is not particularly limited in the expansion rate defined by Formula (4) as long as the restoration rate defined by Formula (1) is high. Examples of the upper limit of the expansion rate defined by Formula (4) are 15% or less, 13% or less, 10% or less, or 5% or less, and examples of the lower limit thereof are more than 0%, 1% or more, 2% or more, 5% or more, 10% or more, or 13% or more. The expansion rate defined by Formula (4) in the artificial hair fiber according to the embodiment may be, for example, more than 0% and 17% or less, more than 0% and 15% or less, 2% or more and 15% or less, 5% or more and 15% or less, 5% or more and 13% or less, 5% or more and 10% or less, more than 0% and 10% or less, or more than 0% and 5% or less. However, from the viewpoint of reducing the feeling of strangeness between the artificial hair and human hair, it is preferable that the expansion rate defined by Formula (4) is small.

The artificial hair fiber according to the embodiment may have a contraction rate C of 17% or less as defined by the following Formula (5). The contraction rate C defined by Formula (5) is an index of contraction characteristics when the artificial hair fiber is dried from the wet state.

Contraction rate C={1−(length of artificial fibroin fiber when dried from wet state/length of artificial fibroin fiber in wet state)}×100(%) Formula (5):

The artificial hair fiber according to the embodiment is not particularly limited in the contraction rate C defined by Formula (5) as long as the restoration rate defined by Formula (1) is high. Examples of the upper limit of the contraction rate C defined by Formula (5) are 15% or less, 13% or less, 10% or less, and 5% or less, and examples of the lower limit thereof are more than 0%, 1% or more, 2% or more, 5% or more, 10% or more, and 13% or more. The contraction rate C defined by Formula (5) in the artificial hair fiber according to the embodiment may be, for example, more than 0% and 17% or less, more than 0% and 15% or less, 2% or more and 15% or less, 5% or more and 15% or less, 5% or more and 13% or less, 5% or more and 10% or less, more than 0% and less than 10%, or more than 0% and less than 5%. However, from the viewpoint of reducing the feeling of strangeness between the artificial hair and the human hair, it is preferable that the contraction rate C defined by Formula (5) be small.

The artificial hair fiber according to the embodiment is a fiber having a contraction history in which it has been irreversibly contracted by contact with water after spinning, and preferably has a contraction rate A of 2% or more defined by the following Formula (2). The contraction rate A defined by Formula (2) is an index showing characteristics of the raw material fiber. When the contraction rate A defined by Formula (2) is 2% or more, the artificial hair fiber has a behavior similar to that of human hair during wetting and drying.

Contraction rate A={1−(length of fiber irreversibly contracted by contact with water after spinning/length of fiber after spinning and before contact with water)}×100(%) Formula (2):

The contraction rate A defined by Formula (2) may be 2.5% or more, 3% or more, 3.5% or more, 4% or more, 4.5% or more, 5% or more, 5.5% or more, 6% or more, 10% or more, 15% or more, 20% or more, or 25% or more. The upper limit of the contraction rate A defined by Formula (2) is not particularly limited and may be 80% or less, 60% or less, 40% or less, 20% or less, 10% or less, 7% or less, 6% or less, 5% or less, 4% or less, or 3% or less.

The artificial hair fiber according to the embodiment is a fiber having a contraction history in which it has been irreversibly contracted by contact with water after spinning and then further contracted by drying, and the contraction rate B defined by the following Formula (3) is preferably more than 7%. The contraction rate B defined by Formula (3) is an index showing characteristics of the raw material fiber. When the contraction rate B defined by Formula (3) is more than 7%, the artificial hair fiber has a behavior similar to that of human hair during wetting and drying.

Contraction rate B={1−(length of fiber irreversibly contracted by contact with water after spinning and then further contracted by drying/length of fiber after spinning and before contact with water)}×100(%) Formula (3):

The contraction rate B defined by Formula (3) may be 10% or more, 15% or more, more than 25%, 32% or more, 40% or more, 48% or more, 56% or more, 64% or more, or 72% or more. The upper limit of the contraction rate B defined by Formula (3) is not particularly limited, but it is usually 80% or less.

The artificial hair fiber according to the embodiment preferably has recesses extending in the fiber axis direction in the surface. Gloss is curbed and an exterior similar to that of human hair can be realized by providing recesses (grooves) in the surface. For example, when the raw material fiber is spun, a spinning method which is a wet spinning method, a method of slowing down a desolvation speed (for example, a method of adding a solvent of a doping solution to a coagulating solution), a method described in International Publication No. 2016/201369 (for example, a method of lengthening a residence time in a coagulation bath (60 seconds or more), and a method of changing a solvent ratio in the coagulation bath) can be adopted as a method of providing a recess in the surface of the artificial hair fiber.

The artificial hair fiber according to the embodiment may have a heat contraction rate of 4% or less as defined by the following Formula (6).

Heat contraction rate={1−(length of artificial fibroin fiber when heated to 160° C./length of artificial fibroin fiber before heating)}×100(%) Formula (6):

Since the artificial hair fiber according to the embodiment is made of the artificial fibroin fiber containing a modified fibroin, a softening point is higher than that of a synthetic fiber, and a temperature thereof is comparable to that of human hair. Therefore, a heat contraction rate at 160° C. (a heat contraction rate defined by Formula (6)) is small. A hot air temperature of a hair dryer is 120 to 140° C., and an optimum temperature for using a curling iron is 170° C. or less. Since the heat contraction rate defined by Formula (6) is small (for example, 4% or less), damage when a hair dryer or a curling iron is used can be curbed. The heat contraction rate defined by Formula (6) may be 3% or less, or 2.5% or less.

Since the artificial hair fiber according to the embodiment is made of the artificial fibroin fiber containing a modified fibroin, it can also be manufactured as an animal-free material (excluding animal-derived components).

The artificial hair fiber according to the embodiment is suitably used as artificial hair (for example, a wig, an extension) because it can be stably supplied and occurrence of a feeling of strangeness in comparison with human hair can be curbed therewith.

### EXAMPLES

Hereinafter, the present invention will be described in more detail based on Examples and the like. However, the present invention is not limited to the following Examples.

### Test Example 1: Manufacture and Evaluation of Artificial Hair Fiber (1)

(1) Manufacture of Modified Fibroin

A modified fibroin (PRT399) having the amino acid sequence set forth in SEQ ID NO: 18, a modified fibroin (PRT380) having the amino acid sequence set forth in SEQ ID NO: 12, a modified fibroin (PRT410) having the amino acid sequence set forth in SEQ ID NO: 13, and a modified fibroin (PRT799) having the amino acid sequence set forth in SEQ ID NO: 15 were designed.

Nucleic acids encoding the four designed modified fibroins were synthesized. In each of the nucleic acids, an NdeI site was added to the 5′-terminal, and an EcoRI site was added downstream of a stop codon. These four types of nucleic acids were cloned into a cloning vector (pUC118). Then, each of the nucleic acids was cut out by a restriction enzyme treatment with NdeI and EcoRI, and then was recombined into a protein expression vector pET-22b (+) to obtain an expression vector.

Escherichia coli BLR (DE3) was transformed with the obtained pET-22b (+) expression vector. The transformed Escherichia coli was cultured in 2 mL of an LB medium containing ampicillin for 15 hours. A culture solution was added to 100 mL of a seed culture medium (Table 4) containing ampicillin so that OD₆₀₀ was 0.005. A temperature of the culture solution was maintained at 30° C., and flask culture was carried out until the OD₆₀₀ reached 5 (about 15 hours) to obtain a seed culture solution.

TABLE 4
Seed culture medium

| Reagent | Concentration (g/L) |
|---|---|
| Glucose | 5.0 |
| KH₂PO₄ | 4.0 |
| K₂HPO₄ | 9.3 |
| Yeast Extract | 6.0 |
| Ampicillin | 0.1 |

The seed culture solution was added to a jar fermenter to which 500 mL of a production medium (Table 5) was added so that the OD₆₀₀ was 0.05. While the temperature of the culture solution was maintained at 37° C., the culture was carried out at a constant pH of 6.9. Moreover, a dissolved oxygen concentration in the culture solution was maintained at 20% of a dissolved oxygen saturation concentration.

TABLE 5
Production medium

| Reagent | Concentration (g/L) |
|---|---|
| Glucose | 12.0 |
| KH₂PO₄ | 9.0 |
| MgSO₄ · 7H₂O | 2.4 |
| Yeast Extract | 15 |
| FeSO₄ · 7H₂O | 0.04 |
| MnSO₄ · 5H₂O | 0.04 |
| CaCl₂ · 2H₂O | 0.04 |
| Adecanol | 0.1 (mL/L) |
| (Adeka, LG-295S) | |

Immediately after glucose in the production medium was completely consumed, a feed solution (glucose 455 g/l L, Yeast Extract 120 g/l L) was added at a rate of 1 mL/min. While the temperature of the culture solution was maintained at 37° C., the culture was carried out at a constant pH of 6.9. The culture was carried out for 20 hours while the dissolved oxygen concentration in the culture solution was maintained at 20% of the dissolved oxygen saturation concentration. Then, 1 M isopropyl-β-thiogalactopyranoside (IPTG) was added to the culture solution to a final concentration of 1 mM to induce the expression of the desired modified fibroin. When 20 hours had passed after the addition of IPTG, the culture solution was centrifuged, and cells were collected. SDS-PAGE was performed using the cells prepared from the culture solutions before and after the addition of IPTG, and the expression of the desired modified fibroin was confirmed by appearance of a band corresponding to a size of the desired modified fibroin depending on the addition of IPTG

The cells collected 2 hours after the addition of IPTG were washed with 20 mM Tris-HCl buffer solution (pH 7.4). The washed cells were suspended in 20 mM Tris-HCl buffer solution (pH 7.4) containing about 1 mM of PMSF, and the cells were disrupted with a high-pressure homogenizer (GEA Niro Soavi Co.). The disrupted cells were centrifuged to obtain a precipitate. The obtained precipitate was washed with 20 mM Tris-HCl buffer solution (pH 7.4) until it became highly pure. The washed precipitate was suspended in 8 M guanidine buffer solution (8 M guanidine hydrochloride, 10 mM sodium dihydrogen phosphate, 20 mM NaCl, 1 mM Tris-HCl, pH 7.0) to have a concentration of 100 mg/mL, and was dissolved by stirring at 60° C. for 30 minutes with a stirrer. After dissolution, dialysis was performed with water using a dialysis tube (a cellulose tube 36/32 manufactured by Sanko Junyaku Co., Ltd.). A white agglutinating protein obtained after the dialysis was recovered by centrifugation. Water was removed from the recovered aggregated protein with a lyophilizer to obtain a lyophilized powder of the desired modified fibroin.

(2) Manufacture of Raw Material Fiber

Dimethyl sulfoxide (DMSO) in which lithium chloride was dissolved to 4.0% by mass was used as a solvent, and the lyophilized powder of the modified fibroin was added thereto to have a concentration of 18% by mass or 24% by mass (refer to Table 6) and was dissolved for 3 hours using a shaker. Then, insoluble matter and bubbles were removed to obtain a modified fibroin solution.

The obtained modified fibroin solution was used as a dope solution (a spinning dope solution), and a spun and drawn raw material fiber was manufactured by dry-wet spinning using a spinning device according to the spinning apparatus 10 shown in FIG. 6. In the spinning apparatus 10 shown in FIG. 6, the used spinning device includes a second undrawn yarn manufacturing device (a second bath) between the undrawn yarn manufacturing device 2 (a first bath) and the wet heat drawing device 3 (a third bath). The conditions for dry-wet spinning were as follows.

Extrusion nozzle diameter 0.2 mm

Solutions and temperatures in the first to third baths: refer to Table 6

Total drawing ratio: refer to Table 6

Drying temperature: 60° C.

TABLE 6

| | Dope solution | | | | | | | | Total |
|---|---|---|---|---|---|---|---|---|---|
| | | Concen- | First bath | | Second bath | | Third bath | | drawing |
| | Modified | tration | | Tem. | | Tem. | | Tem. | ratio |
| | fibroin | (mass %) | Solution | (° C.) | Solution | (° C.) | Solution | (° C.) | (times) |
| Manufacturing | PRT799 | 24 | 100% | −5 | 100% | 16 | Water | 17 | 1 |
| example 1 | | | methanol | | methanol | | | | |
| Manufacturing | | | | | | | | | 2 |
| example 2 | | | | | | | | | |
| Manufacturing | | | | | | | | | 3 |
| example 3 | | | | | | | | | |
| Manufacturing | | | | | | | | | 4 |
| example 4 | | | | | | | | | |
| Manufacturing | | 18 | | | | | | | 1 |
| example 5 | | | | | | | | | |
| Manufacturing | | | | | | | | | 2 |
| example 6 | | | | | | | | | |
| Manufacturing | | | | | | | | | 3 |
| example 7 | | | | | | | | | |
| Manufacturing | | | | | | | | | 4 |
| example 8 | | | | | | | | | |
| Manufacturing | PRT410 | 24 | | −11 | | 14 | | | 1 |
| example 9 | | | | | | | | | |
| Manufacturing | | | | | | | | | 2 |
| example 10 | | | | | | | | | |
| Manufacturing | | | | | | | | | 3 |
| example 11 | | | | | | | | | |
| Manufacturing | | | | | | | | | 4 |
| example 12 | | | | | | | | | |
| Manufacturing | PRT399 | | | | | | | | 1 |
| example 13 | | | | | | | | | |
| Manufacturing | | | | | | | | | 2 |
| example 14 | | | | | | | | | |
| Manufacturing | | | | | | | | | 3 |
| example 15 | | | | | | | | | |
| Manufacturing | PRT380 | | | | | 11 | | | 1 |
| example 16 | | | | | | | | | |
| Manufacturing | | | | | | | | | 2 |
| example 17 | | | | | | | | | |
| Manufacturing | | | | | | | | | 3 |
| example 18 | | | | | | | | | |
| Manufacturing | | | | | | | | | 4 |
| example 19 | | | | | | | | | |

(3) Manufacture of Artificial Hair Fiber and Evaluation of Contraction Rate a and Contraction Rate B

Artificial fibroin fibers (artificial hair fibers) were manufactured by subjecting the raw material fibers obtained in Manufacturing examples 1 to 19 to a contact step in which they were brought into contact with water or performing a drying step in which they were dried at room temperature after the contact step had been performed.

<Evaluation of Contraction Rate A in Contact Step>

A plurality of raw material fibers having a length of 30 cm were cut out from the wound materials of the raw material fibers obtained in Manufacturing examples 1 to 19. The plurality of raw material fibers were bundled to obtain a raw material fiber bundle having a fineness of 150 denier. A 0.8 g lead sinker was mounted on each of the raw material fiber bundles, and in that state, each of the raw material fiber bundles was immersed in water at the temperatures shown in Tables 7 to 10 for 10 minutes (the contact step). Then, a length of each of the raw material fiber bundles was measured in the water. The length of the raw material fiber bundle in the water was measured with the 0.8 g lead sinker mounted on the raw material fiber bundle to eliminate shrinkage of the raw material fiber bundle. Next, the contraction rate A (%) was calculated for each of the raw material fibers according to the following Formula (2). In Formula (2), L0 indicates the length of the fiber after spinning and before contact with water, which is 30 cm here. Similarly, in Formula (2), Lw indicates the length of fiber irreversibly contracted due to contact with water after spinning, and here is the length of each of the raw material fiber bundles measured in the water.

Contraction rate A={1−(Lw/L0)}×100(%) Formula (2):

<Evaluation of Contraction Rate B in Drying Step>

After the contact step, the raw material fiber bundle was taken out of the water. The raw material fiber bundle taken out was dried at room temperature for 2 hours (the drying step) with the 0.8 g lead sinker mounted thereon, and thus the artificial fibroin fiber (the fiber for artificial hair) was obtained. After drying, a length of each of the artificial fibroin fiber bundles was measured. Next, the contraction rate B (%) was calculated for each of the artificial fibroin fibers according to the following Formula (3). In Formula (3), L0 indicates the length of the fiber after spinning and before the contact with the water, which is 30 cm here. Similarly, in Formula (3), Lwd indicates the length of the fiber irreversibly contracted by the contact with the water after spinning and then further contracted by drying, and here is the length of each of the artificial fibroin fiber bundles measured after drying.

Contraction rate B={1−(Lwd/L0)}×100(%) Formula (3):

The results are shown in Tables 7 to 10.

TABLE 7

| | | Water | Contraction | Contraction |
|---|---|---|---|---|
| Raw material fiber/ | | temperature | rate | rate |
| artificial fibroin fiber | | (° C.) | A (%) | B (%) |
| Manufacturing | 24 wt % | 20 | 0.0 | 7.8 |
| example 1 | PRT799x1 | | | |
| Manufacturing | 24 wt % | | −1.2 | 10.3 |
| example 2 | PRT799x2 | | | |
| Manufacturing | 24 wt % | | 7.2 | 21.2 |
| example 3 | PRT799x3 | | | |
| Manufacturing | 24 wt % | | 13.5 | 26.3 |
| example 4 | PRT799x4 | | | |
| Manufacturing | 18 wt % | | −2.3 | 9.5 |
| example 6 | PRT799x2 | | | |
| Manufacturing | 18 wt % | | 6.0 | 19.7 |
| example 7 | PRT799x3 | | | |
| Manufacturing | 18 wt % | | 14.3 | 27.5 |
| example 8 | PRT799x4 | | | |
| Manufacturing | 24 wt % | 40 | −5.3 | 7.2 |
| example 2 | PRT799x2 | | | |
| Manufacturing | 24 wt % | | 8.7 | 21.3 |
| example 3 | PRT799x3 | | | |
| Manufacturing | 24 wt % | | 14.5 | 26.0 |
| example 4 | PRT799x4 | | | |
| Manufacturing | 18 wt % | | −4.3 | 7.3 |
| example 6 | PRT799x2 | | | |
| Manufacturing | 18 wt % | | 6.2 | 18.3 |
| example 7 | PRT799x3 | | | |
| Manufacturing | 18 wt % | | 16.0 | 28.7 |
| example 8 | PRT799x4 | | | |
| Manufacturing | 24 wt % | 60 | 6.8 | 21.0 |
| example 3 | PRT799x3 | | | |
| Manufacturing | 24 wt % | | 15.0 | 27.5 |
| example 4 | PRT799x4 | | | |
| Manufacturing | 18 wt % | | −1.5 | 10.7 |
| example 6 | PRT799x2 | | | |
| Manufacturing | 18 wt % | | 3.3 | 18.2 |
| example 7 | PRT799x3 | | | |
| Manufacturing | 18 wt % | | 16.2 | 29.0 |
| example 8 | PRT799x4 | | | |

TABLE 8

| | | Water | Contraction | Contraction |
|---|---|---|---|---|
| Raw material fiber/ | | temperature | rate | rate |
| artificial fibroin fiber | | (° C.) | A (%) | B (%) |
| Manufacturing | 24 wt % | 20 | −2.3 | 8.7 |
| example 10 | PRT410x2 | | | |
| Manufacturing | 24 wt % | | 4.7 | 16.7 |
| example 11 | PRT410x3 | | | |
| Manufacturing | 24 wt % | | 10.3 | 22.3 |
| example 12 | PRT410x4 | | | |
| Manufacturing | 24 wt % | 40 | 4.7 | 17.5 |
| example 11 | PRT410x3 | | | |
| Manufacturing | 24 wt % | | 11.5 | 24.0 |
| example 12 | PRT410x4 | | | |
| Manufacturing | 24 wt % | 60 | 2.0 | 16.5 |
| example 11 | PRT410x3 | | | |
| Manufacturing | 24 wt % | | 10.8 | 25.0 |
| example 12 | PRT410x4 | | | |

TABLE 9

| | | Water | Contraction | Contraction |
|---|---|---|---|---|
| Raw material fiber/ | | temperature | rate | rate |
| artificial fibroin fiber | | (° C.) | A (%) | B (%) |
| Manufacturing | 24 wt % | 20 | −3.5 | 7.6 |
| example 13 | PRT399x1 | | | |
| Manufacturing | 24 wt % | | 3.7 | 12.5 |
| example 14 | PRT399x2 | | | |
| Manufacturing | 24 wt % | | 7.0 | 16.8 |
| example 15 | PRT399x3 | | | |
| Manufacturing | 24 wt % | 40 | 3.0 | 12.7 |
| example 14 | PRT399x2 | | | |
| Manufacturing | 24 wt % | | 7.3 | 16.7 |
| example 15 | PRT399x3 | | | |
| Manufacturing | 24 wt % | 60 | 3.3 | 9.3 |
| example 14 | PRT399x2 | | | |
| Manufacturing | 24 wt % | | 6.8 | 14.2 |
| example 15 | PRT399x3 | | | |

TABLE 10

| | | Water | Contraction | Contraction |
|---|---|---|---|---|
| Raw material fiber/ | | temperature | rate | rate |
| artificial fibroin fiber | | (° C.) | A (%) | B (%) |
| Manufacturing | 24 wt % | 20 | −1.1 | 9.4 |
| example 16 | PRT380x1 | | | |
| Manufacturing | 24 wt % | | 2.7 | 13.3 |
| example 17 | PRT380x2 | | | |
| Manufacturing | 24 wt % | | 7.0 | 17.7 |
| example 18 | PRT380x3 | | | |
| Manufacturing | 24 wt % | | 10.0 | 20.2 |
| example 19 | PRT380x4 | | | |
| Manufacturing | 24 wt % | 40 | 3.3 | 14.2 |
| example 17 | PRT380x2 | | | |
| Manufacturing | 24 wt % | | 7.7 | 19.0 |
| example 18 | PRT380x3 | | | |
| Manufacturing | 24 wt % | | 12.0 | 22.0 |
| example 19 | PRT380x4 | | | |
| Manufacturing | 24 wt % | 60 | 2.7 | 14.3 |
| example 17 | PRT380x2 | | | |
| Manufacturing | 24 wt % | | 8.2 | 20.3 |
| example 18 | PRT380x3 | | | |
| Manufacturing | 24 wt % | | 12.0 | 23.2 |
| example 19 | PRT380x4 | | | |

### Test Example 2: Manufacture and Evaluation of Artificial Hair Fiber

(1) Manufacture of Modified Fibroin

A modified fibroin (PRT799) having the amino acid sequence set forth in SEQ ID NO: 15, a modified fibroin (PRT918) having the amino acid sequence set forth in SEQ ID NO: 37, a modified fibroin (PRT917) having the amino acid sequence set forth in SEQ ID NO: 43, and a modified fibroin (PRT1028) having the amino acid sequence set forth in SEQ ID NO: 44 were designed.

Nucleic acids encoding the four designed modified fibroins were synthesized. In each of the nucleic acids, an NdeI site was added to the 5′-terminal, and an EcoRI site was added downstream of a stop codon. These five types of nucleic acids were cloned into a cloning vector (pUC118). Then, each of the nucleic acids was cut out by a restriction enzyme treatment with NdeI and EcoRI, and then was recombined into a protein expression vector pET-22b (+) to obtain an expression vector.

Escherichia coli BLR (DE3) was transformed with the obtained pET22b (+) expression vector. The transformed Escherichia coli was cultured in 2 mL of an LB medium containing ampicillin for 15 hours. A culture solution was added to 100 mL of a seed culture medium (Table 11) containing ampicillin so that OD₆₀₀ was 0.005. A temperature of the culture solution was maintained at 30° C., and flask culture was carried out until the OD₆₀₀ reached 5 (about 15 hours) to obtain a seed culture solution.

TABLE 11
Seed culture medium (per 1 L at the start of culture)

| | |
|---|---|
| Glucose | 5 g |
| KH₂PO₄ | 4 g |
| K₂HPO₄ | 10 g |
| Yeast Extract | 6 g |

Ampicillin was added to a final concentration of 100 mg/L to obtain a seed culture medium.

The seed culture solution was added to a jar fermenter to which 500 mL of a production medium (Table 12) was added so that the OD₆₀₀ was 0.05, and transformed Escherichia coli was inoculated. While the temperature of the culture solution was maintained at 37° C., the culture was carried out at a constant pH of 6.9. Further, a dissolved oxygen concentration in the culture solution was maintained at 20% of a dissolved oxygen saturation concentration.

TABLE 12
Production medium (per 1 L at the start of culture)

| | |
|---|---|
| Glucose | 12 g |
| KH₂PO₄ | 9 g |
| MgSO₄ · 7H₂O | 2.4 g |
| Yeast Extract | 15 g |
| FeSO₄ · 7H₂O | 40 mg |
| MnSO₄ · 5H₂O | 40 mg |
| CaCl₂ · 2H₂O | 40 mg |
| GD-113 | 0.1 mL |
| (antifoaming agent) | |

Immediately after glucose in the production medium was completely consumed, a feed solution (glucose 455 g/l L, Yeast Extract 120 g/l L) was added at a rate of 1 mL/min. While the temperature of the culture solution was maintained at 37° C., the culture was carried out at a constant pH of 6.9. Further, the culture was carried out for 20 hours, while the dissolved oxygen concentration in the culture solution was maintained at 20% of the dissolved oxygen saturation concentration. Then, 1 M isopropyl-β-thiogalactopyranoside (IPTG) was added to the culture solution to a final concentration of 1 mM to induce the expression of the target protein. When 20 hours had passed after the addition of IPTG, the culture solution was centrifuged, and the cells were collected. SDS-PAGE was performed using the cells prepared from the culture solutions before and after the addition of IPTG, and the expression of the target protein was confirmed by appearance of a band of a target protein size depending on the addition of IPTG

The cells collected 2 hours after the addition of IPTG were washed with 20 mM Tris-HCl buffer solution (pH 7.4). The washed cells were suspended in 20 mM Tris-HCl buffer solution (pH 7.4) containing about 1 mM PMSF, and the cells were disrupted with a high-pressure homogenizer (GEA Niro Soavi Co.). The disrupted cells were centrifuged to obtain a precipitate. The obtained precipitate was washed with 20 mM Tris-HCl buffer solution (pH 7.4) until it became highly pure. The washed precipitate was suspended in 8 M guanidine buffer solution (8 M guanidine hydrochloride, 10 mM sodium dihydrogen phosphate, 20 mM NaCl, 1 mM Tris-HCL pH 7.0) to have a concentration of 100 mg/mL, and was dissolved by stirring at 60° C. for 30 minutes with a stirrer. After dissolution, dialysis was performed with water using a dialysis tube (a cellulose tube 36/32 manufactured by Sanko Junyaku Co., Ltd.). A white agglutinated protein obtained after the dialysis was recovered by centrifugation. Water was removed with a lyophilizer, and a lyophilized powder was recovered.

(2) Manufacture of Raw Material Fiber

DMSO in which lithium chloride was dissolved to 4% by mass was used as a solvent, and the lyophilized powder of the modified fibroin was added thereto to have a concentration of 24% by mass. After dissolution with an aluminum block heater at 90° C. for 1 hour, insoluble matter and bubbles were removed to obtain a dope solution (a spinning dope solution).

The dope solution was filled into a reserve tank and was discharged from a monohole nozzle having a diameter of 0.3 mm into a coagulation bath containing 100% by mass of methanol (a coagulation bath at a temperature of 12° C.) using a gear pump. After coagulation, washing and drawing were performed in the coagulation bath having methanol of 100% by mass. After washing and drawing, drying was performed using a dry heat plate (at a drying temperature of 80° C.), and the obtained raw yarn (the raw material fiber) was wound up. The drawing ratio was 6 times.

(3) Manufacture of Artificial Hair Fiber

Each of the raw material fibers was bundled to have a length of about 30 cm to obtain a raw material fiber bundle having a fineness of 150 denier. A 0.8 g lead sinker was mounted on each of the raw material fiber bundles, and each of the raw material fiber bundles was immersed in water at 40° C. for 10 minutes to contract it (the contact step) in that state, was removed from the water and dried at room temperature for 2 hours with the 0.8 g lead sinker mounted thereon (the drying step), and thus the artificial fibroin fibers (the artificial hair fibers) of Examples 1 to 4 having different types of proteins were obtained.

(4) Evaluation of Artificial Hair Fibers (Water Flexibility)

A length of each of the artificial fibroin fibers of Examples 1 to 4 obtained in (3) in a dry state (a length of each of the artificial fibroin fibers before they are brought into the wet state) was measured. Next, the 0.8 g lead sinker was mounted on each of the artificial fibroin fibers, and the raw material fiber bundle was immersed in water at 40° C. for 10 minutes in that state. Then, a length of each of the artificial fibroin fibers (the length of each of the artificial fibroin fibers in the wet state) was measured in the water. The length measurement of each of the artificial fibroin fibers in the water was carried out with the 0.8 g lead sinker mounted thereon to eliminate the shrinkage of each of the artificial fibroin fibers. Then, each of the artificial fibroin fibers taken out of the water was dried at room temperature for 2 hours with the 0.8 g lead sinker mounted thereon. After drying, the length of each of the artificial fibroin fibers (the length of the artificial fibroin fibers when dried from the wet state) was measured. The restoration rate, the expansion rate and the contraction rate C of each of the artificial fibroin fibers were calculated from obtained measured values according to the following Formulas (1), (4) and (5).

Restoration rate=(length of artificial fibroin fiber when dried from wet state/length of artificial fibroin fiber before wet state)×100(%) Formula (1):

Expansion rate={(length of artificial fibroin fiber in wet state/length of artificial fibroin fiber before wet state)−1}×100(%) Formula (4):

Contraction rate C={1−(length of artificial fibroin fiber when dried from wet state/length of artificial fibroin fiber in wet state)}×100(%) Formula (5):

For comparison, the above-described operations were carried out on synthetic fibers (Comparative example 1: Nylon, Comparative example 2: Polyester), and the restoration rate, the expansion rate and the contraction rate C were calculated.

For the artificial fibroin fibers of Examples 1 and 2, the above-described operation was repeated, and the restoration rate, the expansion rate, and the contraction rate C in the second cycle were calculated. The results are also shown in Table 13.

TABLE 13

| | | First cycle | | | Second cycle | | |
|---|---|---|---|---|---|---|---|
| | | Expansion | Contraction | Restoration | Expansion | Contraction | Restoration |
| | | rate (%) | rate C (%) | rate (%) | rate (%) | rate C (%) | rate (%) |
| Example 1 | PRT799 | 15.2 | 14.1 | 99 | 16.4 | 14.1 | 100 |
| Example 2 | PRT918 | 6.6 | 6.7 | 99.5 | 6.2 | 5.8 | 100 |
| Example 3 | PRT917 | 2.3 | 4 | 98.2 | — | — | — |
| Example 4 | PRT1028 | 4.9 | 5.6 | 99 | — | — | — |
| Comparative | Nylon | 0 | 0.22 | | — | — | — |
| example 1 | | | | | | | |
| Comparative | Polyester | 0.43 | 0 | 0 | — | — | — |
| example 2 | | | | | | | |

As shown in Table 13, the artificial fibroin fibers containing the modified fibroin (Examples 1 to 4) have the same characteristics as those of human hair that they expand in the wet state and then return to their original length when dried (a restoration rate of 98.2 to 100%). In addition, these characteristics are maintained even after repeated wetting and drying (Examples 1 and 2).

(5) Evaluation of Artificial Hair Fibers (SEM Observation)

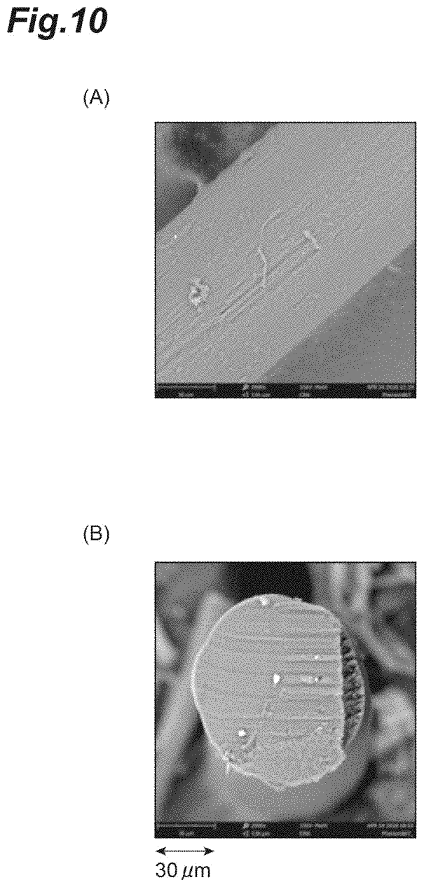

FIG. 10 is a scanning electron microscope (SEM) photograph of the artificial fibroin fiber (PRT918) of Example 2. FIG. 10(A) is an SEM photograph of a surface structure (a skin layer). FIG. 10(B) is an SEM photograph of an internal structure (a cutting surface). It can be seen that the recess is formed in the fiber axis direction.

(6) Evaluation of Artificial Hair Fibers (Heat Contraction)

A heat contraction rate at 160° C. was measured for the artificial fibroin fiber (PRT918) of Example 2 using a thermomechanical analyzer (model number: TMA4000SE, distributor: NETZSCH JAPAN Co., Ltd.). The measurement conditions were as follows.

Fiber length: 10 mm

Temperature rise rate: 10° C./min

Holding time: 1 min

Target temperature: 230° C.

Tension load: 0.1 g

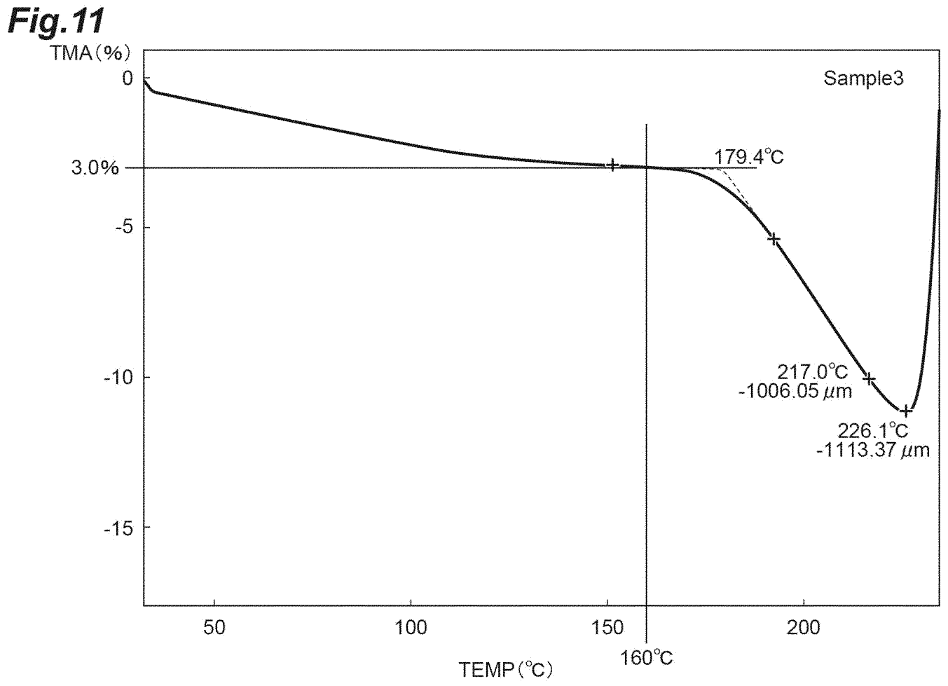

The measurement results are shown in FIG. 11. The heat contraction rate calculated according to the following Formula (6) was 3%.

Heat contraction={1−(length of artificial fibroin fiber when heated to 160° C./length of artificial fibroin fiber before heating)}×100(%) Formula (6):

### REFERENCE SIGNS LIST

1 Extrusion device
2 Undrawn yarn manufacturing device
3 Wet heat drawing device
4 Drying device
6 Dope solution
10 Spinning apparatus
20 Coagulating solution tank
21 Drawing bath
36 Raw material fiber
38 Artificial hair fiber
40 Manufacturing apparatus
42 Feed roller
44 Winder
46 Water bath
48 Dryer
54 Heater
56 Tension roller
58 Hot roller
60 Processing device
62 Drying device
64 Dry heat plate

1. An artificial hair fiber comprising:
an artificial fibroin fiber containing a modified fibroin,
wherein the artificial hair fiber expands when being in a wet state and contracts when being dried from the wet state.

2. The artificial hair fiber according to claim 1, wherein a restoration rate defined by the following Formula (1) is 95% or more;
Restoration rate=(length of artificial fibroin fiber when dried from wet state/length of artificial fibroin fiber before wet state)×100(%) (1),

3. The artificial hair fiber according to claim 1, wherein:
the artificial fibroin fiber is a fiber having a contraction history in which the fiber is irreversibly contracted by contact with water after spinning, and
a contraction rate A defined by the following Formula (2) is 2% or more;
Contraction rate A={1−(length of fiber irreversibly contracted by contact with water after spinning/length of fiber after spinning and before contact with water)}×100(%) (2),

4. The artificial hair fiber according to claim 1, wherein:
the artificial fibroin fiber is a fiber having a contraction history in which the fiber is irreversibly contracted by contact with water after spinning and then further contracted by drying, and
a contraction rate B defined by the following Formula (3) is more than 7%;
Contraction rate B={1−(length of fiber irreversibly contracted by contact with water after spinning and then further contracted by drying/length of fiber after spinning and before contact with water)}×100(%) (3),

5. The artificial hair fiber according to claim 1, wherein the modified fibroin is a modified spider silk fibroin.

6. The artificial hair fiber according to claim 1, wherein a recess which extends in a fiber axis direction is provided in a surface.

7. The artificial hair fiber according to claim 1, wherein an expansion rate defined by the following Formula (4) is 17% or less;
Expansion rate={(length of artificial fibroin fiber in wet state/length of artificial fibroin fiber before wet state)−1}×100(%) (4),

8. The artificial hair fiber according to claim 1, wherein a contraction rate C defined by the following Formula (5) is 17% or less;
Contraction rate C={1−(length of artificial fibroin fiber when dried from wet state/length of artificial fibroin fiber in wet state)×100(%) (5),

9. The artificial hair fiber according to claim 1, wherein a heat contraction rate defined by the following Formula (6) is 4% or less;
Heat contraction rate={1−(length of artificial fibroin fiber when heated to 160° C./length of artificial fibroin fiber before heating)}×100(%) (6),

10. The artificial hair fiber according to claim 1, wherein the artificial hair fiber contains substantially no residual stress generated by drawing in a spinning process.

11. A method for manufacturing an artificial hair fiber, the method comprising:
a contraction step in which a raw material fiber after spinning and before contact with water is brought into contact with water to be irreversibly contracted, then dried and further contracted,
wherein the raw material fiber contains a modified fibroin.

12. The method according to claim 11, wherein the raw material fiber is a fiber having a contraction rate A of 2% or more defined by the following Formula (2);
Contraction rate A={1−(length of fiber irreversibly contracted by contact with water after spinning/length of fiber after spinning and before contact with water)}×100(%) (2),

13. The method according to claim 11 or 12, wherein the raw material fiber is a fiber having a contraction rate B of more than 7% defined by the following Formula (3);
Contraction rate B={1−(length of fiber irreversibly contracted by contact with water after spinning and then further contracted by drying/length of fiber after spinning and before contact with water)}×100(%) (3),

14. The method according to claim 11, wherein, in the contraction step, substantially all residual stress in the raw material fiber generated by drawing in a spinning process is released.

15. The method according to claim 11, wherein the contraction step is performed without relaxing the raw material fiber.

16. The method according to claim 11, wherein the raw material fiber is formed by introducing a spinning dope solution containing the modified fibroin and a solvent into a coagulating solution, removing the solvent from the spinning dope solution and coagulating the spinning dope solution.

17. The method according to claim 11, wherein the modified fibroin is a modified spider silk fibroin.

18. An artificial hair comprising the artificial hair fiber according to claim 1.
